(19) United States
(12) Patent Application Publication
(10) Pub. No.: US 2026/0171215 A1
(43) Pub. Date: Jun. 18, 2026

(54) PSYCHOLOGICAL COUNSELING PROVISION SYSTEM AND METHOD THEREOF

(71) Applicant: ANOTHER SIGHT CO., LTD., Seoul (KR)

(72) Inventor: Yeon Ju HONG, Seoul (KR)

(73) Assignee: ANOTHER SIGHT CO., LTD., Seoul (KR)

(21) Appl. No.: 18/725,940

(22) PCT Filed: Jan. 9, 2023
(86) PCT No.: PCT/KR2023/000363

§ 371 Date: Jul. 1, 2024

(30) Foreign Application Priority Data
Jan. 13, 2022 (KR) 10-2022-0004970
Jan. 13, 2022 (KR) 10-2022-0004971
Jan. 13, 2022 (KR) 10-2022-0004972
Jan. 13, 2022 (KR) 10-2022-0004973
Jan. 13, 2022 (KR) 10-2022-0004974
Nov. 29, 2022 (KR) 10-2022-0162509

(51) Int. Cl. G16H 20/70; G16H 10/20; G16H 10/60; G16H 50/20; G16H 50/30

(52) U.S. Cl. CPC G16H 20/70; G16H 10/20; G16H 10/60; G16H 50/20; G16H 50/30

(57) ABSTRACT

Disclosed are a system for providing psychological counseling and a method therefor. That is, according to the present invention, when a preset theme practice menu is selected from an app execution result screen, in situations where there is a spectator or in situations where there is no spectator focusing on situations where a user of a terminal shows interest, it is possible to easily confirm a user's psychological state in relation to a specific situation in which the user is interested by performing group counseling or group training through a metaverse chat room with a free theme or discussion theme as a topic.

### TECHNICAL FIELD

The present invention relates to a system for providing psychological counseling and a method therefor, and more particularly, to a system for providing psychological counseling that, when a preset theme practice menu is selected from an app execution result screen, in situations where there is a spectator or in situations where there is no spectator focusing on situations in which a user of a terminal shows interest, performs group counseling or group training through a metaverse chat room with a free theme or discussion theme as a topic, and a method therefor.

### BACKGROUND ART

Social anxiety refers to the vague feeling of anxiety that instability in social order has on all members of society.

Approximately 70% of the total population experiences mild social anxiety symptoms such as presentation anxiety, social phobia, and gaze phobia, and 1 out of 10 people have social anxiety disorder (SAD). If SAD is taken lightly and not actively treated, it develops into chronic conditions and causes more and more difficulties in social life.

### DISCLOSURE

### Technical Problem

An object of the present invention provides a system for providing psychological counseling that, when a preset theme practice menu is selected from an app execution result screen, in situations where there is a spectator or in situations where there is no spectator focusing on situations in which a user of a terminal shows interest, performs group counseling or group training through a metaverse chat room with a free theme or discussion theme as a topic, and a method therefor.

Another object of the present invention provides a system for providing psychological counseling that, when a preset my report menu is selected from an app execution result screen, provides analysis results according to the performance of functions such as topic talk, role-playing, and theme practice in relation to a user of a terminal, and a participation list related to a chat list in which the corresponding user participates, and a method therefor.

Still another object of the present invention provides a system for providing psychological counseling that, when a preset topic talk menu is selected from an app execution result screen, performs a chat function with other users of terminals focusing on a specific topic in which a user of a terminal is interested and keywords related to the specific topic, and provides chat results based on self-evaluation and counterpart evaluation, and a method therefor.

Yet another object of the present invention provides a system for providing psychological counseling that, when a preset role-playing menu is selected from an app execution result screen, performs role-playing with other users of terminals focusing on a specific topic in which a user of a terminal is interested and keywords related to the specific topic, and provides chat results based on self-evaluation and counterpart evaluation, and a method therefor.

Yet another object of the present invention provides a system for providing psychological counseling that, when a preset theme practice menu is selected from an app execution result screen, provides feedback related to situations in which a user of a terminal is interested either alone or in collaboration with other users of terminals and provides chat results based on self-evaluation and counterpart evaluation according to the feedback, and a method therefor.

Yet another object of the present invention provides a system for providing psychological counseling that, when a preset people diary menu is selected from an app execution result screen, records emotions felt by a user of a terminal in a specific situation during the day in a diary format, and a method therefor.

### Technical Solution

According to one aspect of the present invention, a system for providing psychological counseling includes: a terminal that displays a room creation screen corresponding to a selected room creation menu when a preset room creation menu is selected from an app execution result screen displayed on the terminal, displays a spectator type setting screen corresponding to a selected theme practice item when a preset theme practice item is selected from the room creation screen displayed on the terminal, displays a theme setting screen corresponding to a selected no spectator item when a preset no spectator item is selected from the spectator type setting screen displayed on the terminal, displays a questionnaire setting screen for receiving self-introduction or question content when a preset question-and-answer item is selected from the displayed theme setting screen, displays a detailed theme setting screen to which received questionnaire setting information is applied when a registration item displayed on one side of the questionnaire setting screen is selected after receiving questionnaire setting information including a plurality of input values corresponding to a plurality of input items included in the questionnaire setting screen displayed on the terminal, and is linked with a server to create a new question-and-answer theme practice chat room corresponding to received detailed theme setting information when a room creation item displayed on one side of the detailed theme setting screen is selected after receiving the detailed theme setting information including a plurality of other input values corresponding to a plurality of other input items included in the detailed theme setting screen displayed on the terminal; and the server that manages a chat function in the question-and-answer chat room related to the theme practice.

A waiting screen may include at least one of a type of the chat room, a topic, a chat room name, a keyword, a nickname of a user in the chat room, a level, and the number of chats, and the level may be calculated based on the number of chats and the received evaluation results.

The terminal may enter the created question-and-answer theme practice chat room to display a waiting screen, and when a room leader who opens the corresponding chat room selects a preset start menu while meeting the number of people set in the created question-and-answer theme practice chat room, may be linked with one or more other terminals owned by each of one or more other users in the question-and-answer chat room related to the theme practice and the server to perform a chat function in relation to a specific theme of the question-and-answer chat room.

According to another aspect of the present invention, a method of providing psychological counseling includes: when a preset room creation menu is selected from an app execution result screen displayed on a terminal, displaying, by the terminal linked with a server, a room creation screen corresponding to the selected room creation menu; when a preset theme practice item is selected from the room creation screen displayed on the terminal, displaying, by the terminal linked with the server, a spectator type setting screen corresponding to the selected theme practice item; when a preset no spectator item is selected from the spectator type setting screen displayed on the terminal, displaying, by the terminal linked with the server, a theme setting screen corresponding to the selected no spectator item; when a preset question-and-answer item is selected from the displayed theme setting screen, displaying, by the terminal, a questionnaire setting screen for receiving self-introduction or question content; after receiving questionnaire setting information including a plurality of input values corresponding to a plurality of input items included in the questionnaire setting screen displayed on the terminal, when a registration item displayed on one side of the questionnaire settings screen is selected, displaying, by the terminal, a detailed theme setting screen to which the received questionnaire setting information is applied; after receiving detailed theme setting information including a plurality of other input values corresponding to a plurality of other input items included in the detailed theme setting screen displayed on the terminal, when the room creation item displayed on one side of the detailed theme setting screen is selected, creating, by the terminal linked with the server, a new question-and-answer theme practice chat room corresponding to the received theme detailed setting information and entering the created question-and-answer theme practice chat room to display a waiting screen; and when a room leader who opens the corresponding chat room selects a preset start menu while the number of people set in the generated question-and-answer theme practice chat room is met, performing, by the terminal linked with one or more other terminals owned by each of one or more other users in the question-and-answer chat room related to the theme practice and the server, a chat function in relation to a specific theme of the question-and-answer chat room.

According to still another aspect of the present invention, a method of providing psychological counseling includes: when a preset room creation menu is selected from an app execution result screen displayed on a terminal, displaying, by the terminal linked with a server, a room creation screen corresponding to the selected room creation menu; when a preset theme practice item is selected from the room creation screen displayed on the terminal, displaying, by the terminal linked with the server, a spectator type setting screen corresponding to the selected theme practice item; when a preset spectator item is selected from the spectator type setting screen displayed on the terminal, displaying, by the terminal linked with the server, other theme setting screens corresponding to the selected spectator item; when any one preset theme item is selected from the displayed other theme setting screens, displaying, by the terminal, other detailed theme setting screens for receiving a room title, the number of participants in training, or the maximum number of spectators; after receiving other detailed theme setting information including a plurality of other input values corresponding to a plurality of other input items included in the other detailed theme setting screens displayed on the terminal, when the room creation item displayed on one side of the other detailed theme setting screens is selected, creating, by the terminal linked with the server, a new free theme chat room or a discussion theme chat room corresponding to the received other theme detailed setting information and entering the created free theme chat room or discussion theme chat room to display a waiting screen; and when a room leader who opens the free theme chat room or the discussion theme chat room selects a preset start menu while the number of people set in the created free theme chat room or discussion theme chat room is met, performing, by the terminal linked with one or more other terminals owned by each of one or more other users in the free theme chat room or the discussion theme chat room related to the theme practice and server, a chat function in relation to a specific theme of the free theme chat room or the discussion theme chat room.

The method may further include: when the theme practice related to the question-and-answer chat room ends, performing, by the terminal linked with the server, a self-evaluation function for the user of the terminal itself; generating, by the server, a chat result for the user of the terminal according to the self-evaluation function performed on the terminal, and transmitting the generated chat result to the terminal; and displaying, by the terminal, the chat result transmitted from the server.

The method may further include: updating, by the server, a “free expression” index, an anxiety index, and a level related to the user based on the chat result generated in relation to the user of the terminal.

In relation to one or more chat rooms in which the user of the terminal participates for a preset period or all chat rooms in which the user of the terminal participates, the “free expression” index may be calculated by adding up an average score self-evaluated by the user after performing the chat function in the chat room in which the user participates and results of a counterpart's evaluation of the user in the chat room in which the user participates, and the anxiety index may represent an average value of anxiety self-evaluated by the user after performing the chat function in the chat room in which the user participates.

The method may further include: when the theme practice related to the free theme chat room or the discussion theme chat room ends, performing, by the terminal linked with the server, a self-evaluation function on the user of the terminal itself and a counterpart evaluation function on one or more other users participating in the free theme chat room or the discussion theme chat room; generating, by the server, chat results for each user participating in the chat room and transmitting each of the generated chat results for each user to the terminal depending on the performance of the self-evaluation function and the counterpart evaluation function performed by each of the plurality of terminals participating in the free theme chat room or the discussion theme chat room; and displaying, by the terminal, the chat result transmitted from the server.

A method of providing psychological counseling includes: when a terminal is invited to a metaverse chat room created in an expert terminal, entering, by the terminal, the metaverse chat room to display a waiting screen; displaying, by the terminal, a questionnaire transmitted from the expert terminal via a server; receiving, by the terminal, questionnaire response information including a plurality of input values corresponding to a plurality of input items included in the questionnaire displayed on the terminal, and transmitting the received questionnaire response information to the expert terminal; displaying, by the terminal, a response result to a questionnaire related to the questionnaire response information transmitted from the expert terminal in response to questionnaire response information related to the previously transmitted questionnaire; performing, by the terminal linked with the server and the expert terminal, one of a psychological counseling function, a speech lesson function, and a mentor function with an expert through a metaverse chat room created by the expert terminal; and displaying, by the terminal, feedback information transmitted from the expert terminal after a chat function ends.

### Advantageous Effects

According to the present invention, when a preset theme practice menu is selected from an app execution result screen, in situations where there is a spectator or in situations where there is no spectator focusing on situations in which a user of a terminal shows interest, by performing group counseling or group training through a metaverse chat room with a free theme or discussion theme as a topic, it is possible to easily confirm a user's psychological state in relation to a specific situation in which the user is interested.

In addition, according to the present invention, when a preset “my report” menu is selected from an app execution result screen, by providing analysis results according to the performance of functions such as topic talk, role-playing, and theme practice in relation to a user of a terminal, and a participation list related to a chat list in which the corresponding user participates, it is possible for a user to easily confirm a user's psychological state for each situation and the user's overall psychological state in relation to chat.

In addition, according to the present invention, when a preset topic talk menu is selected from an app execution result screen, by performing a chat function with other users of terminals focusing on a specific topic in which a user of a terminal is interested and keywords related to the specific topic, and providing psychological counseling based on self-evaluation and counterpart evaluation, it is possible to easily confirm a user's psychological state in relation to the specific topics in which the user is interested and the keywords.

In addition, according to the present invention, when a preset role-playing menu is selected from an app execution result screen, by performing role-playing with other users of terminals focusing on a specific topic in which a user of a terminal is interested and keywords related to the specific topic, and providing psychological counseling based on self-evaluation and counterpart evaluation, it is possible to easily confirm a user's psychological state according to the role-playing in relation to the specific topics in which the user is interested and the keywords.

In addition, according to the present invention, when a preset theme practice menu is selected from an app execution result screen, by providing feedback related to situations in which a user of a terminal is interested either alone or in collaboration with other users of terminals and providing chat results based on self-evaluation and counterpart evaluation according to the feedback, it is possible to easily confirm a user's psychological state in relation to the specific situation in which the user is interested.

In addition, according to the present invention, when a preset people diary menu is selected from an app execution result screen, by recording emotions felt by a user of a terminal in a specific situation during the day in a diary format, it is possible to conveniently organize a change in a user's emotional state by date.

### DESCRIPTION OF DRAWINGS

FIG. 1 is a block diagram illustrating a configuration of a system for providing psychological counseling according to an embodiment of the present invention.

FIGS. 2 to 6 are flowcharts illustrating a method of providing psychological counseling according to an embodiment of the present invention.

FIGS. 7 to 80 are diagrams illustrating examples of screen configurations of a terminal according to an embodiment of the present invention.

### BEST MODE

It is to be noted that technical terms used in the present invention are used in order to describe only specific exemplary embodiments rather than restricting the present invention. In addition, unless indicated otherwise in the present invention, it is to be understood that all the technical terms used in the present invention are construed with meanings that are generally understood by those skilled in the art rather than with excessively comprehensive meanings or excessively reduced meanings. In addition, when the technical terms used in the present invention are wrongly technical terms that do not accurately indicate the technical spirit of the present invention, it is to be understood that the terms can be replaced with the technical terms understood by those skilled in the art. Further, the general terms used in the present invention should be understood according to the terms defined by the dictionary or according to the context and should not have excessively reduced meanings.

In addition, singular forms used in the present invention are intended to include plural forms unless the context clearly indicates otherwise. In the present invention, it is to be noted that the terms “configured of,” “including,” or the like, are not to be construed as necessarily including several components or several steps described in the present invention and some of the above components or steps may not be included or additional components or steps may be construed as being further included.

Terms including an ordinal number such as “first,” “second,” or the like, used in the present invention may be used to describe various components. However, these components are not limited to these terms. These terms are used only in order to distinguish one component from another component. For example, a “first” component may be named a “second” component, and vice versa, without departing from the scope of the present invention.

Hereinafter, exemplary embodiments of the present invention will be described in detail with reference to the accompanying drawings. The same reference numerals will be used to describe the same or like components throughout the drawings, and overlapping description of the same components will be omitted.

Further, when it is decided that the detailed description of the known art related to the present invention may obscure the gist of the present invention, the detailed description thereof will be omitted. In addition, it is to be noted that the accompanying drawings are provided only in order to allow the spirit of the present invention to be easily understood and are not to be interpreted as limiting the spirit of the present invention.

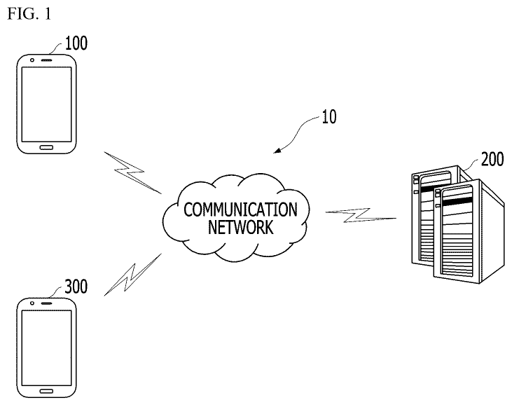

FIG. 1 is a block diagram illustrating a configuration of a system 10 for providing psychological counseling according to an embodiment of the present invention.

As illustrated in FIG. 1, a system 10 for providing psychological counseling includes a terminal 100, a server 200, and an expert terminal 300. Not all of the components of the system 10 for providing psychological counseling illustrated in FIG. 1 are essential components, and the system 10 for providing psychological counseling may be implemented with more components than those illustrated in FIG. 1, or may be implemented with fewer components.

The terminal 100 and the expert terminal 300 may be applied to various terminals such as a smart phone, a portable terminal, a mobile terminal, a foldable terminal, a personal digital assistant (PDA), a portable multimedia player (PMP) terminal, a telematics terminal, a navigation terminal, a personal computer, a laptop computer, a slate PC, a tablet PC, an ultrabook, wearable devices (e.g., including a smartwatch, a smart glass, a head mounted display (HMD), etc.), a Wibro terminal, an Internet protocol television (IPTV) terminal, a smart TV, a digital broadcasting terminal, an audio video navigation (AVN) terminal, an audio/video (A/V) system, a flexible terminal, a digital signage device, etc.

The server 200 may be implemented in the form of a web server, a database server, a proxy server, etc. In addition, the server 200 may be equipped with a network load balancing mechanism or one or more of various types of software that allows the server 200 to operate on the Internet or other networks, and thus may be implemented as a computerized system. In addition, the network may be an http network, a private line, an Intranet, or any other network. Furthermore, the connection between the terminal 100, the server 200, and the expert terminal 300 may be connected to a secure network to prevent data from being attacked by any hacker or other third party. In addition, the server 200 may include a plurality of database servers, and these database servers may be implemented in such a way that they are separately connected to the server 200 through any type of network connections, including distributed database server architecture.

The terminal 100, the server 200, and the expert terminal 300 may each include a communication unit (not illustrated) for performing a communication function with other terminals, a storage unit (not illustrated) for storing various types of information and programs (or applications), a display unit (not illustrated) for displaying various types of information and program execution results, an audio output unit (not illustrated) for outputting voice information corresponding to the various types of information and program execution results, a control unit (not illustrated) for controlling various components and functions of each terminal, etc.

The terminal 100 communicates with the server 200, the expert terminal 300, etc. In this case, the terminal 100 may be a terminal owned by a user who uses the chat room related to topic talk, role-playing (or role play), theme practice, etc., provided by the server 200 to anonymously perform chat functions with other users or perform counseling functions with experts.

In addition, the terminal 100 is linked with the server 200 to register as a user (or client) who receives, through a dedicated app and/or website provided by the server 200, a chat room search function, a chat function/user-to-user chat function, a counseling with expert function/expert search function, a room creation function, a topic talk chat room function, a role-playing chat room function, a theme practice chat room function, a people diary writing and/or management function, a “my report” management function, etc., and register personal information, etc., in the server 200. In this case, the personal information includes an ID, an email address, a password (or passcode), a name, a gender, a date of birth, contact information, an address (or address information), etc.

In addition, the terminal 100 may subscribe a user as a member on the server 200 using SNS account information to which the user of the terminal 100 subscribes or other site account information. Here, the SNS account may be information related to Facebook, Twitter, Instagram, Kakao Story, Naver Blog, etc. In addition, other site accounts may be information related to YouTube, Kakao, Naver, etc. In this case, the dedicated app may be configured as different user interfaces according to a user who receives a chat room search function, a chat function/user-to-user chat function, a counseling with expert function/expert search function, a room creation function, a topic talk chat room function, a role-playing chat room function, a theme practice chat room function, a people diary writing and/or management function, a “my report” management function, etc., and an expert (or counselor) who performs a counseling function for a user, a people diary/“my report” check function for a specific user while performing the counseling function, a counseling result writing and management function, a speech lesson function, a mentor function (or mentoring function), a psychological counseling function, an extended reality (XR) chat room (or metaverse chat room) creation and management function, etc.

In addition, when performing the member subscription process, the terminal 100 should complete an authentication function through an identity authentication method (e.g., including a mobile phone, a credit card, an i-PIN, etc.) to complete the member subscription process for the server 200 normally.

In addition, after the member subscription is completed, to use the service provided by the server 200, the terminal 100 installs a dedicated app (or application/application/specific app) provided from the server 200 on the corresponding terminal 100. At this time, examples of the dedicated app include a native app, a mobile web app, a mobile webapp design (RWD), an adaptive web design (AWD), a hybrid app, etc., and may be an app for performing a chat room search function, a chat function/user-to-user chat function, a counseling with expert function/expert search function, a room creation function, a topic talk chat room function, a role-playing chat room function, a theme practice chat room function, a people diary creation and/or management function, a “my report” management function, a user community function for information exchange between users, an expert community function for information exchange between experts, etc.

In addition, after the member subscription is completed, the terminal 100 may display a discount coupon provided by the server 200 through the corresponding dedicated app. In this case, the discount coupon may be a discount coupon including discount information of a certain percentage when using the chat room function, etc., provided by the server 200.

In addition, the terminal 100 executes a dedicated app pre-installed on the terminal 100 and displays an app execution result screen according to the execution of the dedicated app. In this case, the app execution result screen includes an edit menu (or button/item) for screen editing, a search menu (or expert search menu) for searching for the chat room corresponding to the search word or searching for an expert, a notification menu for confirming notices or notification messages, etc., a “my page” menu (or setting menu) for setting a user information or dedicated app function, etc., an advertising display area (or advertising/event display menu) where advertising content or event content is displayed, a subscription menu for regular subscription to chat room service, a user-to-user menu for performing chat functions corresponding to topic talk, role-playing, theme practice, etc. with other users related to a specific topic or situation, an expert interaction menu for performing psychological counseling or speech lesson functions with an expert, a chat room display area (or chat room display menu) for displaying a list of chat rooms opened in relation to a preset category, a filter menu for providing a filter function for the list of opened chat rooms, a view only waiting rooms menu for providing a list of waiting chat rooms, a random participation menu for randomly participating in a chat room among the opened chat rooms, a room creation menu for creating a new chat room of one's choice, a home menu for moving to a home screen/home screen, a people diary menu for writing an emotional state by date related to a user, a “my report” menu for providing analysis results based on performance of a user's chat function, participation list, and cognitive behavior therapy record (or cognitive behavior record), etc. In this case, the user-to-user menu (or user-to-user tab) includes an analysis result item for providing analysis results according to the performance of functions such as topic talk, role-playing, and theme practice in relation to the user of the terminal 100, a participation list item for providing a participation list related to the chat list in which the user participates, etc. In addition, the expert interaction menu (or expert interaction tab) includes a participation list menu for providing a list of people who have performed a psychological counseling function, a speech lesson function, or a mentoring function with an expert in relation to a specific topic or situation, a cognitive behavior therapy record menu for providing expert feedback based on performance of a training function on appropriate chat room methods in relation to the specific topic or situation, etc. In addition, the filter screen includes a topic talk filter item (e.g., interpersonal relationship, daily life, employment/workplace, knowledge sharing, well-being, psychology/MBTI, travel, art, sports, other, etc.) related to topic talk, a role-playing filter item (e.g., including work, dating, daily life, other, etc.) related to role-playing, a theme practice filter item (e.g., including blind dates, phone situations, interview situations, work reports, small-scale presentations, large-scale presentations, professional discussions, pros and cons discussion, other, etc.) related to theme practice, etc. Here, the terminal 100 performs a login procedure when running the dedicated app using the ID and password according to the membership subscription, a barcode or a QR code including the ID, etc., while subscribing as a member in the server 200 that provides the dedicated app to perform one or more functions of the dedicated app (e.g., including a chat room search function, a chat function/user-to-user chat function, a counseling with expert function/expert search function, a room creation function, a people diary writing function, a “my report” checking function, etc.). In this case, the dedicated app may be configured as different user interfaces according to a user who receives a chat room search function, a chat function/user-to-user chat function, a counseling with expert function/expert search function, a room creation function, a topic talk chat room function, a role-playing chat room function, a theme practice chat room function, a people diary writing and/or management function, a “my report” management function, etc., and an expert (or counselor) who receives a counseling function for a user, a people diary/“my report” check function for a specific user while performing the counseling function, a counseling result writing and management function, a speech lesson function, a mentor function (or mentoring function), a psychological counseling function, a psychological counseling function, an XR chat room (or metaverse chat room) creation and management function, etc.

In addition, when a preset search menu is selected from the app execution result screen displayed on the terminal 100, the terminal 100 displays a search screen corresponding to the selected search menu to provide a list of chat rooms for each category that are in a standby state (or in which chat is in progress) to perform the chat functions. Here, the search screen includes a search word input item for receiving search words (e.g., including topic, keyword, etc.) according to the user input, a category item for searching for chat rooms opened for each category (e.g., including topic talk, role-playing, theme practice, etc.), a popular search word display item for displaying popular search words, etc.

In addition, when the search word according to the user input of the terminal 100 is received on the search screen displayed on the terminal 100, the terminal 100 is linked with the server 200 to display one or more chat room lists corresponding to the corresponding search word.

That is, when the search word according to the user input of the terminal 100 is received on the search screen displayed on the terminal 100, the terminal 100 transmits the received search word, the identification information of the terminal 100, etc., to the server 200. Here, the identification information of the terminal 100 includes a mobile directory number (MDN), mobile Internet protocol (IP), mobile media access control (MAC), subscriber identification module (SIM) card unique information, a serial number, etc.

In addition, when the chat room corresponding to the search word is found among one or more chat rooms opened in the server 200, the terminal 100 receives the chat room list corresponding to the search word transmitted from the server 200 in response to the previously transmitted search word, etc., and displays (or outputs) the received chat room list. Here, the chat room list includes the number of searched chat rooms, a type of chat room, a topic, a chat room name, a keyword, a nickname (or screen name) of a user who has entered the corresponding chat room, the number of people currently inside compared to the total number of people, and whether the chat is in progress (e.g., waiting, chatting, chat over, etc.), an entry menu (or no entry menu), etc.

In addition, when the corresponding chat room is not found with the search word among one or more chat rooms opened on the server 200, the terminal 100 receives information indicating that the chat room corresponding to the search word transmitted from the server 200 in response to the previously transmitted search word or the like does not exist (or information indicating that the registered chat room corresponding to the corresponding search word does not exist) and displays (or outputs) information indicating that the chat room corresponding to the received search word does not exist (or information indicating that the registered chat room corresponding to the search word does not exist).

In addition, when a preset notification menu is selected from the app execution result screen displayed on the terminal 100, the terminal 100 displays a notification screen corresponding to the selected notification menu to provide notices or notification messages provided from the server 200, messages (or post message content) transmitted from other users, etc. Here, the notification screen includes information on a sender who transmits notices, messages, etc., a title, content, transmission time information (e.g., including 5 minutes ago, 1 hour ago, 2 hours ago, etc.), deletion items, etc.

In addition, when one message is selected from among one or more messages included in the notification screen displayed on the terminal 100, the terminal 100 displays detailed content on the selected message.

In addition, when a preset “my page” menu (or setting menu) is selected from the app execution result screen displayed on the terminal 100, to set user information, a dedicated app function, etc., the terminal 100 displays a “my page” screen corresponding to the selected “my page” menu. Here, the my page screen includes an image item (or character item), a nickname item, an identification (ID) (or email address) item, a self-introduction item, a chat temperature item, a “my badges” item, a “my psychological test results” item, a “my keywords” item, a chosen expert item, a payment means management item, a notice item, a frequently asked questions item, a 1:1 inquiry item, a stipulation and policy item, a setting item, etc. In this case, the chat temperature item may be calculated based on the number of chats, the received evaluation results, etc.

For example, the chat temperature may increase by 0.5° C. per chat to a preset standard value (e.g., 36.5° C.), and when the received evaluation result is affirmative evaluation/satisfactory, the chat temperature may increase by 0.5° C., and when the received evaluation result is negative/not good, the chat temperature may decrease by 0.5° C.

In addition, the concept of level or points may be applied instead of the chat temperature. That is, for the level or points, the level or the preset number of points may be calculated according to a plurality of preset grades based on the number of chats, the received evaluation results, etc.

In addition, the “my badges” item is provided when other users confirm the personal information of the user of the terminal 100, and includes a preset number of (e.g. 3) badges (or badge information) according to user selection among the badges acquired when the user of the terminal 100 participates in a chat room related to various topics and keywords and performs the chat function normally. Here, the types of badges include topic talk rookie (e.g., participation in topic talk chat room less than 30 times), topic talk intermediate (e.g., participation in topic talk chat room 30 to 90 times), topic talk expert (e.g., participation in topic talk chat room exceeding 90 times), role-playing rookie (e.g., participation in role-playing chat room less than 30 times), role-playing intermediate (e.g., participation in role-playing chat room 30 to 90 times), role-playing expert (e.g., participation in role-playing chat room exceeding 90 times), theme practice rookie (e.g., participation in theme practice chat room less than 30 times), theme practice intermediate (e.g., participation in theme practice chat room 30 to 90 times), theme practice expert (e.g., participation in theme practice chat room exceeding 90 times), confidence/assertiveness rookie (e.g., confident and assertive or confidence/assertiveness score exceeding 80 points and 10 times or more based on evaluation results), confidence/assertiveness intermediate (e.g., confident and assertive or confidence/assertiveness exceeding 80 points and 25 times or more based on evaluation results), confidence/assertiveness expert (e.g., confident and assertive or confidence/assertiveness points exceeding 80 points based on evaluation results), mind control rookie (e.g., good emotion control or emotion control exceeding 80 points and 10 times or more based on the evaluation results), mind control intermediate (e.g., good emotion control or emotion control exceeding 80 points and 25 times or more based on evaluation results), mind control expert (e.g., good emotion control or emotion control exceeding 80 points and 50 times or more based on evaluation results), communication skills/logic/persuasive power rookie (e.g., good at communicating logically or accurate communication exceeding 80 points and 10 times or more), communication skills/logic/persuasive power intermediate (e.g., good at communicating logically or accurate communication exceeding 80 points and 25 times or more), communication skills/logic/persuasive power expert (e.g., good communication or accurate communication exceeding 80 points and 50 times or more), immersion rookie (e.g., good immersion in a situation or immersion exceeding 80 points and 10 times or more based on evaluation results), immersion intermediate (e.g., good immersion in a situation or immersion exceeding 80 points more and 25 times or more based on evaluation results), immersion expert (e.g., good immersion in a situation or immersion exceeding 80 points and 50 times or more based on evaluation results), empathy/listening rookie (e.g., good listening and good empathy, or empathy/listening exceeding 80 points and 10 times or more based on evaluation results), empathy/listening intermediate (e.g., good listening and good empathy, or empathy/listening exceeding 80 points and 25 times or more based on evaluation results), empathy/listening expert (e.g., good listening and good empathy, or empathy/listening exceeding 80 points and 50 times or more based on evaluation results), etc.

In addition, the “my keywords” item is an item for pre-registering keywords that the user of the terminal 100 is mainly interested in, and may use one or more keywords stored in the my keyword item when inputting the search word or searching for an expert.

In addition, the chosen expert item is an item for pre-registering an expert in whom the user of the terminal 100 is interested, and may use one or more experts stored in the chosen expert item when searching for an expert.

In addition, when a preset subscription menu (or pro subscription menu) is selected from the app execution result screen displayed on the terminal 100, the terminal 100 displays a subscription application screen corresponding to the selected subscription menu for regular subscription of the chat room service. Here, the subscription application screen (or subscription screen) includes a subscription period selection item (e.g., including a payment amount for each subscription period, etc.), a payment means selection item (e.g., Kakao Pay, a credit/check card, etc.), a payment amount item, a subscription item, etc.

In addition, after receiving a plurality of input values corresponding to the plurality of input items included in the subscription application screen displayed on the terminal 100, when the subscription item displayed on one side of the subscription application screen is selected, the terminal 100 is linked with the server 200 and the payment server (not illustrated) to perform a payment function for a subscription fee (or cost) corresponding to the subscription period selected by the terminal 100. In this case, the server 200 may perform card payment, an automatic transfer function through linkage with a bank payment account, payment using cash points or cash remaining in the account of the terminal 100 that is subscribed as a member of the server 200, and a payment function through simple payments including Kakao Pay, Naver Pay, etc.

When the payment function fails, the terminal 100 receives payment failure information (e.g., including payment date, payment amount, failure information (e.g., insufficient balance, limit exceeded, etc.), etc.) (or information indicating that the payment has failed) transmitted from the server 200 (or the payment server) and displays (or outputs) the received payment failure information.

In addition, when the payment function is performed normally, the terminal 100 receives a payment function performance result transmitted from the server 200 and displays (or outputs) the received payment function performance results. Here, the result of performing the payment function includes the subscription period, the payment amount information, the payment date and time information, etc.

In addition, according to performing the subscription function, the terminal 100 participates in various chat rooms provided from the server 200 within the corresponding subscription period to perform the chat function, the expert counseling function, etc. In this case, the terminal 100 may participate in a chat room of a preset category (e.g., theme practice) only while the subscription function is being performed.

In addition, when the preset expert search menu is selected from the app execution result screen displayed on the terminal 100, the terminal 100 displays the expert search screen corresponding to the selected expert search menu to perform the psychological counseling, the speech lesson, or the mentor function with an expert. Here, the expert search screen includes a sorting menu for sorting functions in specialized fields related to all, psychological counseling, speech lessons, mentoring, etc., an expert count display menu for displaying the number of registered (or active) experts, an immediately available item for providing a list of specialists who are immediately available for counseling, an expert display item for providing information on an expert, a smart filter application menu for searching for an expert according to detailed conditions, desired counseling date and time information, etc. Here, examples of the expert display item include an expert name, a counseling field (or specialty field) (e.g., psychological counseling, speech lesson, mentoring, etc.), a brief introduction, a photo (or image), a fee per session, a rating (or star rating), review information, immediate counseling availability, popularity (or number of likes), etc. In addition, the smart filter application menu includes an expert type item (e.g., including all, psychological counseling, speech lesson, mentoring, etc.), an expert gender item (e.g., including all, male, female, etc.), an expert level (or badge) item (e.g. including all, general with less than 30 sessions, specialty for whom the number of counseling sessions is 30 to 90, master for whom the number of counseling sessions is 90 or more, etc.), a “Please select the topic you would like to discuss together” item (e.g., including “I have concerns about myself” (e.g., including self-esteem, personality/disposition, value, interest, religion, background, marriage, etc.), “Social life is difficult,” “Interpersonal relationships are difficult,” “I need dream/career advice,” “I want to know about my emotions/psychology,” “I need expert advice,” etc.), a “Please select the situation in which you want to say something well” item (e.g., including a situation of expressing oneself, a work situation, a situation of presenting in front of others, a situation of dealing with a difficult subject, a situation that promotes friendship, a situation where there is conflict with others, a situation of having to express honest feelings, a discussion/persuasion situation, etc.), a “Please select the mentoring field you would like to receive mentoring in” item (e.g., including mentoring about oneself, mentoring for social life, mentoring for dream/careers, etc.) etc. Here, the “Please select the topic you would like to discuss together” item may include “me,” social life, interpersonal relationships, dreams/career, emotions/psychology, experts, etc., and the “Please select the situation in which you want to say something well” item may include “expressing myself,” work situation, presentation situation, difficult object, friendship, conflict situation, emotion expression, discussion/persuasion, etc.

In addition, the terminal 100 receives expert search information including a plurality of other input values corresponding to a plurality of other input items according to the user input of the terminal 100 from the expert search screen displayed on the terminal 100. Here, the expert search information includes an expert type, an expert gender, an expert level, “Please select a topic you would like to talk about (or topic you would like to receive psychological counseling on),” “Please select a situation in which you want to say something well (or speech lesson topic),” “Please select a mentoring field you would like to receive mentoring in (or mentoring topic),” desired counseling date and visual information, etc.

In addition, the terminal 100 transmits the received expert search information, the identification information of the terminal 100, etc., to the server 200.

In addition, when an expert corresponding to the expert search information is not found among one or more experts registered in the server 200, the terminal 100 receives information indicating that an expert corresponding to the expert search information transmitted from the server 200 has not been found in response to the previously transmitted expert search information and displays (or outputs) information indicating that the expert corresponding to the received expert search information has not been found.

In addition, as the search results (or the confirmation results), when the expert corresponding to the expert search information is found among one or more experts registered in the server 200, the terminal 100 receives one or more pieces of expert information corresponding to the expert search information transmitted from the server 200 in response to the previously transmitted expert search information, and displays (or outputs) the received one or more pieces of expert information. Here, the expert information includes an expert name, a counseling field (or specialty field) (e.g., psychological counseling, speech lessons, mentorship, etc.), a brief introduction, a photo (or image), a fee per session, a rating (or star rating), review information, immediate counseling availability, popularity (or number of likes), etc.

In addition, when the specific expert information is selected from one or more pieces of expert information displayed on the terminal 100, the terminal 100 is linked with the server 200 to display the detailed expert information corresponding to the selected specific expert information. Here, the detailed expert information includes an expert name item, a counseling field (or specialty) item (e.g., including psychological counseling, speech lesson, mentoring, etc.), a rating (or star rating) item, a photo (or image) item, a brief introduction item, a usage fee per counseling session, a re-use desire rate of users who received counseling item, a total number of counseling sessions item, a counseling information item, a counselor information item, a review item, etc. In addition, the counseling information item includes a price option item (e.g., including a basic counseling item, a severe anxiety counseling item, etc.), a detailed description item, a location item, a popularity (or number of likes) item, a counseling application item, etc. In addition, the basic counseling item, the severe anxiety counseling item, etc., include the number of counseling sessions, additional composition (e.g., XR chat room training 40%, data analysis 60%, etc.), time (e.g., including 1 hour, etc.), price (e.g., 50,000 won/time), etc. In addition, the counselor information item includes a counselor introduction, a career introduction, a certification introduction, etc. In addition, the review item includes an overall star rating, ratings for each category (e.g., including use of XR, friendliness, analytical skills, empathy and comfort, appropriate advice, etc.), the total review, etc.

In addition, the terminal 100 is linked with the server 200 to perform a counseling reservation function for a specific expert corresponding to the corresponding specific expert information.

That is, when the preset counseling reservation menu displayed on one side of the expert detailed information of the terminal 100 is selected, the terminal 100 is linked with the server 200 and the payment server (not illustrated) to perform the payment function for the preset counseling cost related to the expert. In this case, the server 200 may perform card payment, an automatic transfer function through linkage with a bank payment account, payment using cash points or cash remaining in the account of the terminal 100 that is subscribed as a member of the server 200, and a payment function through simple payments including Kakao Pay, Naver Pay, etc. Here, before/after performing the payment function for the counseling fee, the terminal 100 may schedule the counseling with the corresponding expert, or may automatically reserve (or confirm) the counseling schedule with the expert according to the desired counseling date and time information included in the expert search information.

In addition, the terminal 100 receives the information indicating that the counseling schedule with the corresponding expert transmitted from the server 200 is confirmed, and displays (or outputs) the information indicating that the counseling schedule with the received corresponding expert has been confirmed.

In addition, the terminal 100 is linked with the server 200 to perform a final confirmation function (or final acceptance/confirmation function) for the counseling schedule case newly registered in the expert terminal 300 so that the counseling schedule for the counseling schedule case newly registered in the expert terminal 300 is finally confirmed.

In addition, when a preset expert search menu is selected from the app execution result screen displayed on the terminal 100, the terminal 100 is linked with the server 200 and allows the user of the terminal 100 to confirm whether to perform the preset counseling questionnaire writing function.

In addition, when the user of the terminal 100 does not perform the counseling questionnaire writing function, the terminal 100 displays a preset counseling questionnaire screen before displaying the expert search screen corresponding to the selected expert search menu. Here, the counseling questionnaire screen is for the purpose of confirming the psychological state related to the user of the terminal 100 and may be provided to experts performing the counseling function in the future, and includes a plurality of questions and view items that include “I have difficulty dealing with extreme anxiety right before a presentation,” “If I improve my personality, I will be able to communicate better with others,” “When I speak, others seem to secretly ignore me,” “I want to gain the courage to be honest about myself and my feelings,” “I want to increase the persuasive power of my words when presenting or speaking,” “I feel like my voice or the way I speak is unattractive,” “I want to know how to react well to what my counterpart says when chatting,” “I find it difficult to speak calmly when I am embarrassed during presentations and interviews,” “I am worried that I may be doing a job that is not suitable for me,” “I need someone to have fun chats about topics I like,” “My confidence is low because I know less than other people,” “I am thirsty for productive self-improvement chat,” etc.

In addition, the terminal 100 receives a plurality of answers according to the user selection of the terminal 100 for the plurality of questions included in the counseling questionnaire screen displayed on the terminal 100, and then transmits the plurality of received answers, the identification information of the terminal 100, etc., to the server 200.

In addition, the terminal 100 transmits the plurality of received answers, etc., to the server 200 and then displays an expert search screen corresponding to the selected expert search menu.

In addition, the terminal 100 is linked with the server 200 to perform the psychological test function.

In addition, when a specific category (e.g., including all, topic talk, role-playing, theme practice, etc.) within the preset conversation menu is selected from the app execution result screen displayed on the terminal 100, the terminal 100 is linked with the server 200 to filter one or more chat rooms corresponding to the selected specific category among the plurality of chat rooms created in the corresponding server 200 and display the filtered one or more chat rooms (or information on the corresponding chat rooms) in the chat room display area.

In addition, when a preset filter menu is selected from the app execution result screen displayed on the terminal 100, the terminal 100 displays a filter screen corresponding to the selected filter menu to provide a filter function for the list of opened chat rooms. Here, the filter screen includes a topic talk filter item (e.g., interpersonal relationship, daily life, employment/workplace, knowledge sharing, well-being, psychology/MBTI, travel, art, sports, other, etc.) related to topic talk, a role-playing filter item (e.g., including work, dating, daily life, other, etc.) related to role-playing, a theme practice filter item (e.g., including blind date, phone situation, interview situation, 1:1 meeting/work report, small-scale presentation, large-scale presentation, professional discussion, other, etc.) related to theme practice, etc.

In addition, after receiving the filter information including the plurality of input values corresponding to the plurality of input items included in the filter screen displayed on the terminal 100, when the applying menu (or confirmation menu) displayed on one side of the filter screen is selected, the terminal 100 is linked with the server 200 to filter the plurality of chat rooms created in the server 200 according to the received filter information and display one or more filtered chat rooms (or information on the corresponding chat room) in the chat room display area.

In addition, when the preset only waiting room view menu is selected from the app execution result screen displayed on the terminal 100, the terminal 100 is linked with the server 200 to filter at least one chat room in which the chat function is not yet being performed (or a waiting state/in waiting) among the plurality of chat rooms created in the server 200 and display at least one filtered chat room (or information on the corresponding chat room) in the chat room display area.

In addition, when a preset random participation menu is selected from the app execution result screen displayed on the terminal 100, the terminal 100 is linked with the server 200 to participate in any one randomly selected chat room among one or more category-specific chat rooms created by one or more other terminals (not illustrated) and perform the chat function related to the corresponding category.

In addition, when a preset room creation menu is selected from the app execution result screen displayed on the terminal 100, to create a new chat room in relation to the category the user wants, the terminal 100 displays the room creation screen corresponding to the selected room creation menu. Here, the room creation screen includes a room type selection menu for selecting a room type, etc. In addition, the room type selection menu includes the topic talk item for free chat between users with similar interests, a role-playing item for practicing expressing emotions and opinions by setting up situations, a theme practice item for developing (or practicing) adaptability and appropriate responsiveness by creating the theme that may cause tension, etc. In this case, the room creation menu may be activated and displayed only on the terminal 100 of a pre-registered (or set) expert terminal 300 or a paid member (or subscription member) for whom chat room creation rights are set.

In addition, when a preset topic talk item is selected from the room creation screen displayed on the terminal 100, the terminal 100 displays a topic talk chat room creation screen corresponding to the selected topic talk item to communicate freely with users with similar interests. Here, the topic talk chat room creation screen includes a topic selection menu (e.g., including interpersonal relationships, daily life, employment/workplace, knowledge sharing, well-being, psychology/MBTI, travel, art, sports, etc.), a keyword selection menu for selecting keywords related to a specific topic selected from the topic selection menu, a room title input menu for selecting a room title, an answer time selection menu for setting an answer time per person (e.g., including 45 seconds, 60 seconds, 90 seconds, etc.), the maximum number of people menu for setting the maximum number of people (e.g., including 2, 3, 4, etc.), etc.

In addition, after receiving a plurality of input values corresponding to the plurality of input items included in the topic talk chat room creation screen displayed on the terminal 100, when the room creation menu (or creation menu) displayed on one side of the topic talk chat room creation screen is selected, the terminal 100 is linked with the server 200 to create (or open) a new topic talk chat room corresponding to the plurality of received input values. Here, the plurality of input values include a topic, a keyword, a room title, an answer time, the maximum number of people, etc. In this case, the topic talk chat room creation may be performed in the terminal 100 that is performing a subscription function in advance or in the terminal 100 that has paid a preset usage amount for each topic to create the topic talk chat room. Here, when the keyword related to the specific topic included in the plurality of input values is used by a plurality of users more than the preset number of times (for example, 10 times), the server 200 may preferentially provide the corresponding keyword (or hashtag) according to an auto-complete function so that other users may easily use the corresponding keyword (or hashtag).

In this way, the terminal 100 may be used for various personal purposes, such as overcoming speaking anxiety by speaking in front of others, relieving isolation and depression by having enjoyable chats and feeling empathy and solidarity with other people, and practicing various presentations, reading discussions, and speaking English, through the topic talk chat room, which allows for maximum free chat between individuals, like real chat, with minimal intervention.

In addition, when a preset role-playing item is selected from the room creation screen displayed on the terminal 100, the terminal 100 displays a role-playing chat room creation screen corresponding to the selected role-playing item to practice expressing emotions and opinions by setting up situations. Here, the role-playing chat room creation screen includes a context setting menu (e.g., including work, dating, daily life, etc.), a room title input menu for selecting a room title, an answer time selection menu for setting an answer time per person (e.g., including 45 seconds, 60 seconds, 90 seconds, etc.), and a maximum number of people menu for setting the maximum number of people (e.g., including 2, 3, 4, etc.).

In addition, after receiving a plurality of other input values corresponding to the plurality of other input items included in the role-playing talk chat room creation screen displayed on the terminal 100, when the room creation menu (or creation menu) displayed on one side of the role-playing chat room creation screen is selected, the terminal 100 is linked with the server 200 to create (or open) a new role playing chat room corresponding to the plurality of received other input values. Here, the plurality of other input values include situation information, a room title, an answer time, the maximum number of people, etc. In this case, the role-playing chat room creation may be performed in the terminal 100 that is performing a subscription function in advance or in the terminal 100 that has paid a preset usage amount for each case to create the role-playing chat room.

In this way, the terminal 100 may allow participants to receive roles and scripts and perform short chats, uses the role-playing chat room in which users, who are response targets, should spontaneously and appropriately respond in turn to allow people who are not good at expressing their emotions (or opinions) due to fear of the gaze and evaluation of others to overcome their anxiety and express themselves appropriately, and help train context understanding through role-playing based on real situations since patients with social anxiety disorder tend to be slow in understanding social context.

In addition, when the preset theme practice item is selected from the room creation screen displayed on the corresponding terminal 100, to develop (or practice) adaptability and appropriate responsiveness by creating the theme that may cause tension, the terminal 100 displays a spectator type setting screen corresponding to the selected theme practice item. Here, the spectator type setting screen includes a no spectator item, a spectator item, etc., for selecting the theme type according to the presence or absence of a spectator.

In addition, when a preset no spectator item is selected from the spectator type setting screen displayed on the terminal 100, to create a theme practice chat room for performing the chat function with the question-and-answer, the terminal 100 displays a theme setting screen corresponding to the selected no spectator item. Here, the theme setting screen (or question theme setting screen) includes a question-and-answer item, a next item, etc. In this case, the chat room without a spectator (or theme practice chat room without a spectator) may be configured to be created in the pre-registered (or set) expert terminal 300 or the terminal 100 of a paid member (or subscription member) where the chat room creation permission is set.

In addition, when the preset question-and-answer item is selected from the theme setting screen displayed on the terminal 100, the terminal 100 displays the questionnaire setting screen for receiving the self-introduction or question content. Here, the questionnaire setting screen may be configured in different forms (or formats) from the perspective of a general user or an expert/trainer, and includes a simple self-introduction/question purpose section, one or more questionnaire items the user would like to ask other participants, a questionnaire addition item, a registration item, etc. Here, the one or more questionnaire items may include at least one basic question (e.g., “Briefly introduce yourself,” “Have you ever participated in outside activities or clubs?” “What kind of work you would like to do at the company?” etc.) that is preset by default. Here, the registration item remains deactivated by default, and may be displayed by switching from the deactivated state to the activated state when the input values for the simple self-introduction/question purpose item, the questionnaire item, etc., are input (or received).

In addition, the terminal 100 receives questionnaire setting information including the plurality of input values corresponding to the plurality of input items included in the questionnaire setting screen displayed on the terminal 100 according to the user input. Here, the questionnaire setting information includes a brief self-introduction/purpose of the question, content of one or more questionnaires to be asked to other participants, etc.

In addition, after receiving the questionnaire setting information from the questionnaire setting screen, when the registration item displayed on one side of the questionnaire setting screen is selected, the terminal 100 displays the detailed theme setting screen to which the received questionnaire setting information is applied (or reflected). Here, the detailed theme setting screens (or other theme practice chat room creation screen/question-and-answer theme setting screens) include a category item (e.g., including blind date practice without a spectator, etc.), a brief self-introduction/question purpose item, one or more questionnaire content items, a room title input item for selecting the room title, a “number of general users who can participate in training” input item (e.g., no, 1, 2, 3, 4 people, etc., male/female) for selecting the number of general users who can participate in training, a “number of experts who can participate in feedback” input item (for example, include 1, 2, 3, etc.) for selecting the number of experts who can participate in feedback, a room creation item, etc. Here, the room creation item remains deactivated by default, and may be displayed by switching from the deactivated state to the activated state when the input values for the category item, the brief self-introduction/question purpose item, the one or more questionnaire content items, the room title input item, the “number of general users who can participate in training” input item, the “number of experts that can participate in feedback” input item, etc., are input (or received).

In addition, the terminal 100 receives other detailed theme setting information including the plurality of other input values corresponding to the plurality of other input items included in the detailed theme setting screen displayed on the terminal 100 according to the user input. Here, the detailed theme setting information includes the category (e.g., question-and-answer without a spectator), brief self-introduction/question purpose, content of one or more questions the user wants to ask other participants, a room title, and the number of general users who can participate in training, the number of experts who can participate in feedback, etc.

In addition, after receiving the detailed theme setting information on the detailed theme setting screen displayed on the terminal 100, when the room creation item (or creation item) displayed on one side of the detailed theme setting screen is selected, the terminal 100 is linked with the server 200 to create (or open) a new theme practice chat room (or question-and-answer theme practice chat room) corresponding to the received detailed theme setting information. In this case, the theme practice chat room creation may be performed in the terminal 100 that is performing a subscription function in advance or in the terminal 100 that has paid a preset usage amount for each topic to create the theme practice chat room. In addition, the terminal 100 displays the waiting screen according to the entry into the chat room. Here, the waiting screen includes the type of the corresponding chat room, the topic, the chat room name, the keyword, the nickname (or chat name) of the user in the corresponding chat room, the chat temperature, and the number of chats (or the number of times the user has participated in the chat room), etc. In this case, the chat temperature may be calculated based on the number of chats, the received evaluation results, etc., and the concept of level or points may be applied instead of the chat temperature. That is, for the level or points, the level or the preset number of points may be calculated according to a plurality of preset grades based on the number of chats, the received evaluation results, etc.

The embodiment of the present invention mainly describes the creation of the question-and-answer theme practice chat room in the terminal 100, but is not limited thereto. The expert terminal 300 may also create the question-and-answer theme practice chat room and invite the corresponding terminal 100.

In addition, when a preset spectator item is selected from the spectator type setting screen displayed on the terminal 100, to create a theme practice chat room in the form of a free theme or discussion theme, the terminal 100 displays other theme setting screens corresponding to the selected spectator item. Here, the other theme setting screen (or free/discussion theme setting screen) includes a free theme item (e.g., including blind date practice item, phone practice item, interview practice item, business report practice item, small-scale presentation practice item, large-scale presentation practice item, singing practice item, etc.), a discussion theme item (e.g., including professional discussion item, pros and cons discussion item, etc.), a registration item, etc.

The embodiment of the present invention mainly describes performing the creation of the chat room with a spectator (or theme practice chat room with a spectator) in the terminal 100, but is not limited thereto, and the chat room with a spectator may also be configured to be created only in the pre-registered (or set) expert terminal 300 where the chat room creation right is set.

In addition, when any one theme item is selected from other theme setting screens displayed on the terminal 100, the terminal 100 displays other detailed theme setting screens for receiving the room title, the number of participants in training, or the maximum number of spectators. Here, the other detailed theme setting screens (or other theme practice chat room creation screens/free/discussion theme setting screen) include a category item (e.g., including blind date practice with a spectator, phone practice with a spectator, etc.), a room title input item for selecting the room title, a “number of general users who can participate in training” input item (e.g., 1, 2, 3, 4 people, etc., male/female) for selecting the number of general users who can participate in training, a “maximum number of spectators” input item (e.g., 5, 10, 20 people, etc.) for selecting the number of users who can be spectate, a room creation item, etc. In this case, when a professional discussion item or pros and cons discussion item included in the discussion theme item in the other theme setting screens is selected, before displaying the other detailed theme setting screens, the terminal 100 may first display a discussion topic setting screen, receive one or more discussion topics (or discussion topic information/content) according to the user input, and then display the other detailed theme setting screens to which one or more discussion topics are applied. Here, when the room creation item is kept in a deactivated state by default, and input values for the category item, the room title input item, the “number of general users who can participate in training” input item, and the “maximum number of spectators” input item, etc., are input (or received), the room creation item may be displayed by switching from a deactivated state to an activated state.

In addition, the terminal 100 receives other detailed theme setting information including a plurality of additional input values corresponding to a plurality of additional input items included in other detailed theme setting screens displayed on the terminal 100 according to the user input. Here, the other detailed theme setting information includes categories (e.g., blind date practice with spectators, phone practice, interview practice, work reporting practice, small-scale presentation practice, large-scale presentation practice, professional discussion, pros and cons discussion, etc.), a discussion topic, a room title, the number of general users who can participate in training, the maximum number of spectators, etc.

In addition, after receiving other theme detailed setting information on other theme detailed setting screens displayed on the terminal 100, when the room creation item (or creation item) displayed on one side of other theme detailed setting screens is selected, the terminal 100 is linked with the server 200 to create (or open) a new theme practice chat room (or free theme practice chat room/discussion theme practice chat room) corresponding to the received other detailed theme setting information and enter the created new theme practice chat room. In this case, the theme practice chat room creation may be performed in the terminal 100 that is performing a subscription function in advance or in the terminal 100 that has paid a preset usage amount for each topic to create the theme practice chat room. In addition, the terminal 100 displays the waiting screen according to the entry into the chat room. Here, the waiting screen includes the type of the corresponding chat room, the topic, the chat room name, the keyword, the nickname (or chat name) of the user in the corresponding chat room, the chat temperature, and the number of chats (or the number of times the user has participated in the chat room), etc. In this case, the chat temperature may be calculated based on the number of chats, the received evaluation results, etc., and the concept of level or points may be applied instead of the chat temperature. That is, for the level or points, the level or the preset number of points may be calculated according to a plurality of preset grades based on the number of chats, the received evaluation results, etc.

The embodiment of the present invention mainly describes the creation of the corresponding free theme practice chat room or discussion theme practice chat room in the terminal 100, but is not limited thereto. The expert terminal 300 may also create the free theme practice chat room or the discussion theme practice chat room and invite the corresponding terminal 100.

In this way, the terminal 100 may provide a place to practice for people who are particularly nervous in specific situations to overcome anxiety and to respond quickly and flexibly through the theme practice chat room to intensively create the specific situations (e.g., including interviews, company dinners, blind dates, etc.) and practice quick and appropriate responses.

In addition, in this way, the terminal 100 may create the topic talk chat room, the role-playing chat room, the theme practice chat room, etc., according to the user request of the terminal 100.

When the chat function is being performed in the chat room created in this way, other general users cannot use the spectator mode for the chat room in which the chat function is being performed, and only an expert who has set up the counseling function in advance in relation to the specific user participating in the corresponding chat room may be allowed to use the spectator mode to perform the monitoring functions for that specific user.

In other words, the present invention is intended to provide a service with the purpose of exposure training, which may be said to be the most important cognitive behavior therapy program for social anxiety disorder, and is configured to give all users the right to participate in a chat room and share their thoughts or opinions with anonymous others and disable a spectator mode for general users in order to provide an opportunity to actively participate in the chat. This prevents people from just watching what other users do and talk about in the chat room, allows them to participate directly, and enables users who feel anxious about giving presentations or talking to people to perform chats through an anonymous avatar so that they may overcome this anxiety as much as possible.

However, when the psychological counselor, the speech lesson expert, or the mentor (or mentor expert) is performing the counseling with the client (or user), the state of being able to spectate the chat may only be allowed when the client needs to observe, diagnose, and train the appearance of the chat through the chat with other users.

In addition, when the terminal 100 participates in the topic talk chat room, the role-playing chat room, the theme practice chat room, etc., the terminal 100 performs the chat function with other users in a similar state (or at a similar level) as the user based on the self-diagnosis results for the user of the terminal 100 according to the artificial intelligence in the server 200.

In addition, when a preset home menu is selected from the app execution result screen displayed on the terminal 100, the terminal 100 displays a preset home screen (or initial screen).

In addition, when the preset people diary menu is selected from the app execution result screen displayed on the terminal 100, the terminal 100 displays the people diary screen corresponding to the selected people diary menu to write the emotional state by date related to the user. Here, the people diary screen includes a date selection menu for selecting a date, when there is a pre-stored memo (or diary/emotional record content) corresponding to the activated (or selected) date, a memo content display area (or memo content display menu) for displaying the corresponding memo content, a detailed emotional record menu for recording detailed emotions, a simple record menu for simply recording emotions, a calendar menu for confirming an emotional record state by date, etc. In this case, the current date (or today's date) in the corresponding terminal 100 may be displayed by default in the date selection item.

In this way, in the case of a general user who does not have social anxiety disorder, the terminal 100 may be configured to write his or her normal schedule (e.g., including time, location, activity details, etc.) and to be used for scheduler purposes through the corresponding people diary (or people diary function/people screen).

In this case, there are in-session exposure and cognitive reconstruction among the most important treatment protocols when treating people with social anxiety disorder, and in the case of the in-session exposure, the terminal 100 may be configured to enable treatment and training using the chat function of the present invention.

In addition, the cognitive reconstruction process explores how users (or patients) cope with situations they face in reality, what automatic thoughts they have, and what cognitive errors they make, and represents leading to new rational responses with the help of experts, and since people with social anxiety usually have characteristics such as negative thinking, behavior that causes errors in thinking, distorted thinking and anxiety, may record “How you behaved in situations you faced in real life,” “What emotions you had and how you expressed them,” activities, emotional state, etc., so they can apply it themselves as a cognitive reconstruction activity.

For example, the terminal 100 may perform recording so that a user can think once more about what automatic thoughts they have about a situation, whether they understood their emotions and expressed them or not, and what their rational response is through questions such as “Where have you been?” “With whom? ”What activities?“ ”How did you feel? “What emotions did you feel?” “How much emotion did you express?” recording emotions, and “How much emotions were expressed.” Above all, when the client later receives counseling with an expert based on people diary records, with the expert's permission, it may be organized so that the expert may explore negative automatic thoughts together and help the client with cognitive reconstruction training to enable the rational thinking.

In addition, the cognitive reconstruction process of the present invention may be performed (or managed) for each user by the learning through the artificial intelligence.

In addition, after a specific date is selected from the people diary screen displayed on the terminal 100 according to the user input (or user selection/touch/control) of the terminal 100, when the preset detailed emotional record menu is selected, the terminal 100 displays a detailed emotional record screen corresponding to the selected detailed emotional record menu. Here, the detailed emotional record screen includes a date selection item for selecting a date, a situation selection item for selecting a specific situation, an emotion selection item for selecting an emotional state felt by a user in relation to the specific situation, a review selection item related to a user's regrets and future resolutions in relation to the specific situation, etc. In addition, examples of the situation selection item include a “Who were you with today?” item (e.g., including family, a date/lover, a friend, work/a coworker, school, a senior/junior, others, etc.) for selecting who on was with, a “What did they do?” item (e.g., including affirmative items such as “They complimented me,” “They empathized with me,” “They comforted me,” “They gave me advice, and “They respected me,“ negative items such as “They ignored me, “They were annoyed, “They were upset, “They were angry, and “We argued,” etc.) for selecting what action the counterpart did, a “What action did I take?” item for selecting what action one took oneself (e.g., including affirmative items such as “I were considerate,” ”I apologized,” “I acknowledged it,” ”I supported them,” and “I helped them,” negative items such as “I was embarrassed,” “I was rambling,” “I was having a hard time,” “I was silent,” “I could not do anything,” and ”I forced a smile,” etc.), etc. In addition, examples of the emotion selection item include a “How did I feel at that time?” item (e.g., including happiness, joy, vitality, confidence, courage, spirit, gratitude, appreciation, passion, inspiration, ecstasy, love, anger/fury, irritation, disbelief, displeasure, disappointment, sadness/depression, anxiety, exhausted, fatigue, frustration, nothingness, etc.) for selecting how the user felt in a corresponding situation, a “Did you express that feeling?” item (e.g., including not expressed, expressed a little, expressed actively, etc.) for selecting whether the user expressed the emotion they felt, a “OO is currently a ‘˜˜˜’ person to me” item (e.g., including affirmative items such as reliable, cute, lovely, and kind, neutral items such as mediocre and average, and negative items such as bad and disgusting, etc.) for selecting how one currently feel about one's counterpart, etc. In addition, the review selection item includes a “Did you feel comfortable talking to that person?” item (e.g., including very uncomfortable, uncomfortable, average, comfortable, very comfortable, etc.) for selecting one's comfort level when talking to a counterpart, a “What do you want to do in the future?” item for selecting one's regrets or future resolutions, a “Please write your own memo to record the situation in more detail at that time,” item, etc.

In this case, when there is detailed emotional record information or simple emotional record information pre-stored in relation to the selected specific date, the terminal 100 may display a detailed emotional record screen including the pre-stored detailed emotional record information in relation to the specific date, or a simple emotional record screen including pre-stored simple emotional record information in relation to the corresponding specific date. Here, when there is simple emotional record information in relation to the selected specific date, the terminal 100 may display an emotional record status screen that further includes a detailed emotional record menu so that the detailed emotions may be recorded for the simple emotional record information along with the simple emotional record information pre-stored in relation to the specific date. In addition, when the detailed emotional record menu displayed on the emotional record status screen is selected, the terminal 100 may display the detailed emotional record screen instead of the simple emotional record information pre-stored in relation to the specific date so that detailed emotion may be recorded, and receive the detailed emotional record information according to the user input.

The embodiment of the present invention describes that, after the date is selected on the people diary screen, when the detailed emotional record menu is selected, the terminal 100 displays the detailed emotional record screen corresponding to the selected detailed emotional record menu in relation to the selected date, but is not limited thereto. When the detailed emotional record menu is selected from the people diary screen, the terminal 100 may display the detailed emotional record screen corresponding to the selected detailed emotional record menu with the current date as the default. In this case, the date displayed on the detailed emotional record screen may be modified to a different date depending on the user input.

In addition, the terminal 100 receives detailed emotional record information including a plurality of input values corresponding to the plurality of input items related to the emotion felt by the user in a specific situation on the previously selected specific date (or specific date) according to the user input of the terminal 100 from the detailed emotional record screen displayed on the terminal 100. Here, the detailed emotional record information includes date information, situation information (e.g., including who else was there, other people's behaviors, one's own behaviors, etc.), and emotional information (e.g., including one's emotional state, whether emotions were expressed, one's current emotions, etc.), review information (e.g., level of comfort in chat, future resolutions, memos for oneself, etc.), etc.

In addition, the terminal 100 is linked with the server 200 to map (or match/link) the received detailed emotional record information with the user information of the terminal 100 and stores (or registers/manages) the mapped information in the corresponding server 200.

That is, the terminal 100 transmits the received detailed emotional record information, the emotional record storage request information, the identification information of the terminal 100, etc., to the server 200. Here, the identification information of the terminal 100 includes a mobile directory number (MDN), mobile Internet protocol (IP), mobile media access control (MAC), subscriber identification module (SIM) card unique information, a serial number, etc.

In addition, the terminal 100 updates and displays the people diary screen related to the corresponding specific date.

That is, the terminal 100 displays the people diary screen to which the detailed emotional record information is applied (or reflected) on the corresponding specific date.

In addition, after a specific date is selected from the people diary screen displayed on the terminal 100 according to the user input (or user selection/touch/control) of the terminal 100, when the preset simple record menu is selected, the terminal 100 displays a simple emotional record screen corresponding to the selected simple record menu. Here, the simple emotional record screen includes a date selection item for selecting a date, a situation description input item for inputting information about who did what and with whom, a mental state item (e.g., uncomfortable, average, comfortable, other, etc.) for selecting what one's state of mind was, etc.

The embodiment of the present invention describes that, after the date is selected on the people diary screen, when the simple record menu is selected, the terminal 100 displays the simple emotional recording screen corresponding to the selected simple record menu in relation to the selected date, but is not limited thereto. When the simple record menu is selected on the people diary screen, the terminal 100 may display the simple emotional recording screen corresponding to the selected simple recording menu with the current date as the default. In this case, the date displayed on the simple emotional record screen may be modified to another date according to the user input.

In addition, the simple emotional record information including a plurality of other input values corresponding to the plurality of other input items related to the emotions felt by the user in a specific situation on the specific date (or specific date) previously selected according to the user input of the terminal 100 on the simple emotional recording screen displayed on the terminal 100 is received. Here, the simple emotional record information includes date information, situation description information (e.g., including who else was there, activity details, etc.), mental state information, etc.

In addition, the terminal 100 is linked with the server 200 to map (or match/link) the received simple emotional record information with the user information of the terminal 100 and stores the mapped information in the corresponding server 200 (or registration/management).

That is, the terminal 100 transmits the received simple emotional record information, the other emotional record storage request information, the identification information of the terminal 100, etc., to the server 200.

In addition, the terminal 100 updates and displays the people diary screen related to the specific date.

That is, the terminal 100 displays the people diary screen to which the simple emotional record information is applied (or reflected) on the corresponding specific date.

An embodiment of the present invention describes that, when the people diary menu is selected from the app execution result screen displayed on the terminal 100, the terminal 100 displays the people diary screen including the detailed emotional record menu and the simple record menu in response to the selected people diary menu, but is not limited thereto. When the people diary menu is selected from the app execution result screen, the terminal 100 may display the detailed emotional record screen corresponding to the detailed emotional record menu set by default in response to the selected people diary menu.

In addition, the embodiment of the present invention mainly describes registering one piece of detailed emotional record information or simple emotional record information on the same date, but is not limited thereto, and the terminal 100 may register a plurality of pieces of detailed emotional record information and simple emotional record information on the same date.

In addition, when a preset calendar menu is selected from the people diary screen displayed on the terminal 100, the terminal 100 displays a calendar screen including an emotional record state by date corresponding to the selected calendar menu to display an emotional record state by date. Here, when the emotional record information (including detailed emotional record information, simple emotional record information, etc.) has been stored on the specific date, the calendar screen includes emotional record states such as images (or emoticons/avatars) representing feeling comfortable, normal, uncomfortable, etc., corresponding to mental state information, which is some of the information within the corresponding emotional record information on the corresponding specific date.

In addition, when a preset “my report” menu is selected from the app execution result screen displayed on the terminal 100, to provide the analysis results and participation list related to the chat room in which the user of the terminal 100 participates or provide the participation list and cognitive behavior therapy record related to the chat room in which the user of the terminal 100 participates with the expert, the terminal 100 displays the “my report” screen corresponding to the selected “my report” menu. Here, the “my report” screen includes an edit item for screen editing, a search item for searching for the chat room corresponding to the search word, a notification item for confirming notices or notification messages, etc., a “my page” item (or setting item) for setting user information or dedicated app functions, etc., a user-to-user item for providing analysis results based on the performance of functions such as topic talk, role-playing, and theme practice in relation to the user of the terminal or a participation list related to the chat list in which the user participates, an expert interaction item for providing a cognitive behavior therapy record, which is a list or feedback from the expert performing the psychological counseling function, the speech lesson function, or the mentoring function with an expert in relation to a specific topic or situation, a content display item (or content display area) for displaying the detailed information according to the user-to-user item or expert interaction item selection, etc. In addition, the user-to-user item (or user-to-user tab) includes an analysis result item for providing analysis results according to the performance of functions such as topic talk, role-playing, and theme practice in relation to the user of the terminal 100, a participation list item for providing a participation list related to the chat list in which the user participates, etc. In addition, the expert interaction item (or expert interaction tab) includes a participation list item for providing a list of people who have performed a psychological counseling function, a speech lesson function, or a mentoring function with an expert in relation to a specific topic or situation, a cognitive behavior therapy record item for providing expert feedback based on performance of a training function on appropriate chat room methods in relation to the specific topic or situation, etc.

In addition, when an analysis result item within a preset user-to-user item is selected from the “my report” screen displayed on the terminal 100, the terminal 100 displays the detailed information corresponding to the selected analysis result menu in the content display area to provide analysis results according to the performance of chat functions such as topic talk, role-playing, and theme practice in relation to the user of the terminal 100. In this case, when the preset “my report” menu is selected from the app execution result screen displayed on the corresponding terminal 100, the terminal 100 may display, by default, an analysis result screen (or the detailed information) corresponding to the analysis result item within the item among users set as default in response to the selected “my report” menu in the content display area. Here, the analysis result item (or the analysis result screen/detailed information) includes a sort item for sorting functions related to all, topic talk, role-playing, theme practice, etc., a “number of times you've participated in the chat room” display item for displaying the number of times the user has participated in the chat room within a preset period, a period display item for displaying the corresponding preset period, a “number of times you've participated in the chat room by category” display item for displaying the number of times the user has participated in the chat room by category, a “free expression” index display item for displaying the “free expression” index calculated in relation to the user, an anxiety index display item for displaying the anxiety index calculated in relation to the user, a detailed score view item for displaying additional detailed information in relation to the “free expression” index or the anxiety index, a relative evaluation display item for displaying information on oneself as evaluated by other users who performed the chat function together, a “my ‘free expression’ score aspect” display item for displaying the “free expression” score aspect, a “my anxiety score pattern” display item for displaying the user's anxiety score pattern, a “my confidence/assertiveness, mind control, communication skills/logic/persuasive power, immersion, empathy/listening skills score aspect” display item for displaying the user's confidence/assertiveness, mind control, communication skills/logic/persuasive power, immersion, empathy/listening skills score aspect, a “topic I have practiced a lot” display item for displaying a topic that the user has practiced a lot in relation to the topic talk, a “keyword that I have practiced a lot” display item for displaying keywords that the user has practiced a lot in relation to the topic talk, a “target I've practiced a lot” display item for displaying what the user has practiced a lot in relation to role-playing, a “situation I've practiced a lot” display item for displaying situations in which the user has practiced a lot in relation to role-playing, a “What emotions am I sensitive to?” display item for displaying what emotions the user is sensitive to in relation to role-playing, a “Which experts I have practiced with a lot?” display item for a displaying indicating with which experts the user has practiced a lot in relation to the theme, a “theme I practiced a lot” display item for displaying a theme that the user has practiced a lot in relation to theme practice, etc. Here, the “free expression” index display item includes an overall rating display item, a “free expression” index overall average display item, a satisfaction graph display item for overall average, a “free expression” index change graph display item by day of the week, a score display item for each item (e.g., including confidence/assertiveness, mind control, communication skills/logic/persuasive power, immersion, empathy/listening skills, etc.), a graph display item of a score change for each item by day of the week, an exponential distribution display item for each topic and situation, a detailed score view item, etc. In addition, the anxiety index display item includes a total rating display item, an anxiety index overall average display item, an anxiety graph display item for the overall average, a graph display item of an anxiety index change by day of the week, etc. In this case, the “free expression” index can be calculated by adding up the average score of the user's self-evaluation after performing the chat function in the chat room (e.g., including the topic talk chat room, the role-playing chat room, the theme practice chat room, etc.) in which the user participates, in relation to one or more chat rooms in which the user of the corresponding terminal 100 participates for a preset period or all chat rooms in which the user of the corresponding terminal 100 participates, and results (or evaluation received) (e.g., +1 for affirmative evaluation/satisfied, −1 for negative evaluation/not great) of evaluation of the user by the counterpart in the chat room in which the user participates. In addition, the anxiety index represents the average value of the anxiety self-evaluated by the user after performing the chat function in the chat room (e.g., including the topic talk chat room, the role-playing chat room, the theme practice chat room, etc.) in which the user participates in relation to one or more chat rooms in which the user of the corresponding terminal 100 participates for a preset period or all chat rooms in which the user of the corresponding terminal 100 participates. In addition, examples of the relative evaluation display item include the reason for receiving a good evaluation, the reason for receiving a bad evaluation, and the satisfaction of chat evaluated by others. In addition, examples of the “my ‘free expression’ score aspect” display item include a “How many people am I with when my free expression score gets high?” item, a “What age group of people am I talking with when I have a high score in ‘free expression’?” item, a “What MBTI type am I talking with when I have a high score in ‘free expression’?” item, a “What gender is the person I'm talking with when I have a high score in ‘free expression’?” item, a “When I talk in a certain place, do I have a high score in free expression?” item, etc. In addition, the “my anxiety score aspect” display item includes a “How many people am I with when my anxiety score gets high?” item, a “What age group of people am I talking with when I have a high score in anxiety?” item, a “What MBTI type am I talking with when I have a high score in anxiety?” item, a “What gender is the person I'm talking with when I have a high score in anxiety?” item, a “When I talk in a certain place, do I have a high score in anxiety?” item, etc. In addition, examples of the “topic that I have practiced a lot” display item include an interpersonal relationship item, a daily life item, an employment/workplace item, a knowledge sharing item, a well-being item, other items, etc. In addition, examples of the “keyword that I have practiced a lot” display item include a boss item, a soccer item, a job preparation item, a presentation preparation item, a health item, other items, etc. In addition, examples of the “target that I have practiced a lot” display item include a husband item, a boss item, a coworker item, a chairman item, a stranger item, other items, etc. In addition, examples of the “situation that I have practiced a lot” display item include a rejection expression item, an affection expression item, an opinion expression item, a gratitude/sorry expression item, a conflict item, other items, etc. In addition, examples of the “What emotions am I sensitive to?” display item include a joy/pleasure item, an empathy item, an appreciation item, a sorry item, a grief/hurt item, an anger/annoyance/rage item, etc. In addition, examples of the “Which experts I have practiced with a lot?” display item include a psychological counselor item, a speech trainer item, a mentor item, etc. In addition, examples of the “theme that I practiced a lot” display item include a blind date item, a phone item, an interview item, a business report item, a small-scale presentation item, a large-scale presentation item, a professional discussion item, a pros and cons discussion item, etc.

In addition, when a preset detailed score view item is selected from the detailed information included in the content display area displayed on the terminal 100, the terminal 100 is linked with the server 200 to display detailed score information corresponding to the selected detailed score view item. Here, the detailed score information (or detailed score screen) includes the sort items for sorting functions related to all, topic talk, role-playing, theme practice, etc., the “number of times you've participated in the chat room” display item, the period display item for displaying the corresponding preset period, the “number of times you've participated in the chat room for each category” display item, the “free expression” index display item, the anxiety index display item, the relative evaluation display item, the “my ‘free expression’ score aspect” display item, the “my anxiety score aspect” display item, the “my confidence/assertiveness, mind control, communication skills/logic/persuasive power, immersion, empathy/listening skills score pattern” display item, the “topic I have practiced a lot” display item, the “keyword that I have practiced a lot” display item, the “target that I have practiced a lot” display item, the “situation that I have practiced a lot” display item, the “Which emotions I am sensitive to?” display item, the “Which experts have I practiced with a lot?” display item, the “theme which I have practiced a lot” display item, etc. In this case, when the detailed score view item related to the “free expression” index is selected, the terminal 100 may display one or more pieces of information related to the “free expression” index among the information included in the detailed score information, and when the detailed score view item related to the anxiety index is selected, the terminal 100 may mainly display one or more other pieces of information related to the anxiety index among the information included in the detailed score information.

That is, when a preset detailed score view item is selected from the detailed information included in the content display area displayed on the terminal 100, the terminal 100 is linked with the server 200 to display the detailed score information corresponding to the selected detailed score view item instead of the “free expression” index display item or anxiety index display item displayed previously.

In addition, when a preset period display item (or date selection item/calendar item) is selected from the “my report” screen displayed on the terminal 100, the terminal 100 displays a calendar screen corresponding to the selected period. Here, the calendar screen includes information on the presence or absence of chat records by date recorded by the user of the terminal 100 within the corresponding period (or corresponding month).

In addition, when a specific date within the calendar screen is selected, if the chat record exists on the corresponding specific date, the terminal 100 displays detailed score information related to the chat record, and if no chat record exists on the corresponding specific date, the terminal 100 displays information indicating that no chat record exists.

In addition, when the participation list item in the preset user-to-user item is selected from the my report screen displayed on the terminal 100, the terminal 100 displays a participation list screen corresponding to the selected participation list item to provide a participation list related to the chat list in which the user of the terminal 100 participates. Here, the participation list screen includes a sort item for sorting functions related to all, topic talk, role-playing, theme practice, etc., a change information display item for providing information on changes in self-evaluation results or other's evaluation results compared to a preset period (or last week), a “number of times you've participated in the chat room” display item for displaying the number of times the user has participated in the chat room within a preset period, an information display item for each chat participation case for providing information related to topic talk, role-playing, theme practice, etc., in which the user participates, etc. In this case, the information display item for each chat participation includes a date information display item of participation in the chat room, a detail view item for displaying the detailed information, a chat room type (or chat room category) display item (e.g., including topic talk, role-playing, theme practice, etc.) for displaying the type of chat room in which the user participates, a chat room topic display item for displaying the topic of the chat room, a chat room name display item for displaying the room title (or chat room name) of the chat room, a keyword display item for displaying the main keywords/hashtags related to the corresponding chat room, a self-evaluation information display item for displaying the self-evaluation results, a received evaluation information display item for displaying the evaluation information received from other users participating in the corresponding chat room, a chat recording display item for the re-listening function of chat content from the chat room, etc.

In addition, when a specific participation list (or a detail view item displayed on one side of the corresponding specific participation list) is selected from one or more participation lists displayed in the information display items for each chat participation in the participation list screen displayed on the terminal 100, the terminal 100 is linked with the server 200 to display the detailed view information related to the selected specific participation list. Here, the detailed view information includes a date information display item of participation in the chat room corresponding to the selected specific participation list, a chat room type (or chat room category) display item for displaying the type of chat room in which the user participates (e.g., including a topic talk chat room, a role-playing chat room, a theme practice chat room, etc.), a chat room topic display item for displaying the topic of the chat room, a chat room name display item for displaying the room title of the chat room, a keyword display item for displaying main keywords/hashtags related to the chat room, a chat participant information display item for displaying information (e.g., including nicknames) of other users participating in the corresponding chat room, a self-evaluation result display item (or self-evaluation information display item) for displaying self-evaluation results, a received evaluation result display item (or a received evaluation information display item) for displaying evaluation results received from other users participating in the chat room, a chat recording display item for re-listening to chat content from the chat room, etc.

In addition, when the participation list item (or other participation list items) in the preset expert interaction item is selected from the “my report” screen displayed on the terminal 100, the terminal 100 displays other participation list screens corresponding to the selected participation list item to provide other participation lists in which the user of the terminal 100 performs a counseling function, a speech lesson function, a mentoring function, etc., with an expert. Here, the other participation list screens include other sort items for sorting functions related to all, psychological counseling, speech, mentoring, etc., other change information display items for providing information on changes in the number of counseling sessions or the number of chat training sessions with an expert or changes of a cognitive behavior therapy record compared to a preset period (or last week), other “number of times you've participated in the chat room” display items for displaying the number of times the user has participated in the chat room within a preset period, an information display item for each expert chat participation case for providing information related to psychological counseling, speech, mentoring, etc. in which the user participates, etc. In this case, examples of the information display item for each expert chat participation case include other chat room type (or chat room category) display items for displaying the type of chat room in which the user participates (e.g., including psychological counseling, speech, mentoring, etc.), another chat room topic display item for displaying the topic of the chat room, a detail view item for displaying detailed information, an expert information display item for displaying an expert's photo and name, a counseling date display item for displaying a counseling date, other keyword display items for displaying main keywords/hashtags related to the corresponding chat room, an expert feedback item for providing expert feedback, a cognitive behavior therapy record sheet item for providing a cognitive behavior therapy record sheet, a written questionnaire item for providing questionnaire content written by a user, a chat room training display item for displaying chat room training content, etc.

In addition, when other specific participation lists (or detail view item displayed on one side of the corresponding other specific participation lists) are selected from one or more other participation lists being displayed in the information display items for each expert participation in other participation list screens displayed on the terminal 100, the terminal 100 is linked with the server 200 to display the detailed information related to the selected other specific participation lists. Here, the detailed information (or detailed counseling result item screen) includes other chat room type (or chat room categories) display items for displaying the types of chat rooms (e.g., including psychological counseling, speech, mentoring, etc.) in relation to the other specific participation lists, other chat room topic display items for displaying the topic of the chat room, an expert information display item for displaying the expert's photo and name, a counseling date display item for displaying the counseling date, other keyword display items for displaying the main keywords/hashtags related to the corresponding chat room, an expert feedback display item for displaying the expert feedback, a cognitive behavior therapy record sheet item for providing the cognitive behavior therapy record sheet before and after training, a questionnaire item written during counseling for providing the type and results of the questionnaire written by the user, a chat room participation record display item for providing the chat room participation record, etc. In this case, the questionnaire item written during counseling may include various types of tests (or questionnaires) such as a self-esteem self-diagnosis test, a communication type test, an anxiety test, etc. In addition, examples of the chat room participation record display item include a date information display item of participation in the chat room, an expert state item for displaying whether the expert is participating in training or observing the expert, a chat room type (or chat room category) display item for displaying the type of chat rooms (e.g., including a topic talk chat room, a role-playing chat room, a theme practice chat room, etc.) in which the user participates, a chat room topic display item for displaying the topic of the chat room, a chat room name display item for displaying the room title of the chat room, a chat participant information display item for displaying information (e.g., including nicknames) of other users participating in the corresponding chat room, a self-evaluation result display item (or self-evaluation information display item) for displaying the self-evaluation results, a received evaluation result display item (or received evaluation information display item) for displaying the evaluation results received from other users participating in the corresponding chat room, a chat recording display item for the re-listening function of the chat content from the chat room, etc.

In addition, when the cognitive behavior therapy record item in the preset expert interaction item is selected from the “my report” screen displayed on the terminal 100, to provide expert feedback according to the user of the terminal 100 performing a training function on an appropriate chat room type in relation to a specific topic or situation, the terminal 100 displays the cognitive behavior therapy record screen corresponding to the selected cognitive behavior therapy record item. Here, the cognitive behavior therapy record screen (or cognitive behavior therapy record sheet screen) includes a cognitive behavior therapy record information display item for each chat participation for writing (or providing) a cognitive behavior therapy record before and after training in relation to a specific topic or situation in which the user participates, a new cognitive behavior therapy record sheet writing item for writing a new cognitive behavior therapy record sheet, etc. In addition, examples of the cognitive behavior therapy record information display item for each chat participation include a cognitive behavior therapy record sheet title display item for displaying a title, a writing and modification item for writing or editing a non-written cognitive behavior therapy record, a result view item for displaying the written cognitive behavior therapy record results, a record date display item for displaying a date the cognitive behavior therapy record is written, a record sheet progress display item for displaying record sheet progress before/after training, a confirmed expert display item for displaying an expert who has confirmed a cognitive behavior therapy record sheet written by a user, etc.

The cognitive behavior therapy record sheet (or “free expression” cognitive behavior therapy record sheet) described in the present invention is self-recorded by the user for the period of counseling and/or training with the psychological counselor, and is a record sheet that aims to help better understand one's own and others'actions and motivations, learn professional problem-solving skills to deal with them, and develop confidence in oneself and one's abilities, and may be written (or recorded/edited/created) both before and after training.

In addition, when the writing and modification item displayed on one side of the specific cognitive behavior therapy record is selected from among one or more cognitive behavior therapy record items displayed in the cognitive behavior therapy record information display item for each chat participation in the cognitive behavior therapy record screen displayed on the terminal 100, the terminal 100 is linked with the server 200 to display the cognitive behavior therapy record input screen corresponding to the selected writing and modification item to edit the cognitive behavior therapy record information related to the selected specific cognitive behavior therapy record. Here, in the case of the pre-training, the cognitive behavior therapy record input screen includes the “What are situations that make you anxious?” item, the “How are you feeling at this time” item (e.g., including anger/fury, irritation, disbelief, discomfort, disappointment, sadness/depression, anxiety, tiredness, fatigue, frustration, bitterness, remorse, jealousy, shame, embarrassment, despondence, outrage, panic, isolation, loneliness, boredom, etc.), the “What are your thoughts in that situation?” item (e.g., including a confidence level, a cognitive error, etc.), the “please correct my wrong thoughts” item (or reasonable response finding item), the behavioral goal setting item, etc. In this case, the types of cognitive errors include black and white (or black and white logic), divination (or fortune teller/catastrophe), ignoring (or ignoring advantages), inference (or emotional inference), name (or naming), mind reading (or mind reading technique), filtering (or mental filtering), obsession (or obsessive burden), uselessness (or useless thoughts), etc. In addition, the cognitive behavior therapy record input screen after training includes a “Please display any area that has improved slightly through training” item (e.g., an improvement-related item, an anxiety-related item, etc.), a “What actually happened related to an area I was worried about before training?” item, a “Please re-evaluate your thoughts before training” item, a “how I feel now” selection item (e.g. happiness, joy, vitality, confidence, courage, spirit, gratitude, appreciation, passion, inspiration, ecstasy, love, anger/rage, irritation, disbelief, displeasure, disappointment, sadness/depression, anxiety, tiredness, fatigue, frustration, etc.), a “What did you learn?” item, etc.

In addition, the terminal 100 receives a plurality of input values corresponding to the plurality of input items included in the cognitive behavior therapy record input screen displayed on the terminal 100 according to the user input. Here, the plurality of input values include the input values before training (e.g., including “What are situations that make you anxious?” content, “How are you feeling at this time” content, “What are your thoughts in that situation?” content, “Please correct my wrong thoughts” content, behavioral goal setting content etc.), the input values after training (e.g., “Please display any area that has improved slightly through training” content, “What actually happened related to an area I was worried about before training?” content, “Please re-evaluate your thoughts before training” content, “how I feel now” content, “What did you learn?” content, etc.).

In addition, the terminal 100 is linked with the server 200 to apply the plurality of received input values to the user information of the terminal 100 and the cognitive behavior therapy record information related to the specific cognitive behavior therapy record case and display the cognitive behavior therapy record screen in the state in which the plurality of input values are applied (or updated).

In addition, when the item for writing a new cognitive behavior therapy record sheet in the cognitive behavior therapy record screen displayed on the terminal 100 is selected, the terminal 100 is linked with the server 200 to display a new cognitive behavior therapy record sheet writing screen corresponding to the selected new cognitive behavior therapy record sheet writing item. Here, the new cognitive behavior therapy record sheet creation screen includes a cognitive behavior therapy record sheet title input item for inputting the cognitive behavior therapy record sheet title, a pre-training or post-training item for selecting pre/post-training, a pre-training input item (e.g., including “What are situations that make you anxious?” “How are you feeling at this time?” “What are your thoughts in that situation?” “Please correct my wrong thoughts,” “Please set action goal,” etc.), a post-training input item (e.g., including “Please display any area that has improved slightly through training,” “What actually happened in relation to an area I was worried about before training?” “Please re-evaluate your thoughts before training,” “How do you feel now?” “What have you learned?” etc.) etc.

In addition, the terminal 100 receives a plurality of other input values corresponding to the plurality of other input items included in the new cognitive behavior therapy record input screen displayed on the terminal 100 according to the user input. Here, the plurality of other input values include the cognitive behavior therapy record sheet title, the input values before training (e.g., including “What are situations that make you anxious?” content, “How are you feeling at this time” content, “What are your thoughts in that situation?” content, “Please correct my wrong thoughts” content, behavioral goal setting content, etc.), the input values after training (e.g., “Please display any area that has improved slightly through training” content, “What actually happened related to an area I was worried about before training” content, “Please re-evaluate your thoughts before training” content, “how I feel now” content, “What did you learn?” content, etc.).

In addition, the terminal 100 is linked with the server 200 to generate the new cognitive behavior therapy record related to the user (or user information) of the terminal 100 based on the plurality of received different input values, apply the plurality of received different input values to the cognitive behavior therapy record information related to the generated new cognitive behavior therapy record case, and display the cognitive behavior therapy record screen in the state in which the plurality of different input values are applied.

In addition, when a chat room related to any one topic talk is selected from one or more chat rooms related to the topic talk displayed in the preset chat room display area among the app execution result screen displayed on the corresponding terminal 100, the terminal 100 is linked with the server 200 to enter (or participate in) the chat room related to the selected topic talk. In this case, the terminal 100 displays the waiting screen according to the entry into the chat room. Here, the waiting screen includes the type of the corresponding chat room, the topic, the chat room name, the keyword (or hashtag), the nickname (or chat name) of the user who enters the corresponding chat room, the chat temperature, and the number of chats (or the number of times the user has participated in the chat room), etc. In this case, the chat temperature may be calculated based on the number of chats, the received evaluation results, etc., and the concept of level or points may be applied instead of the chat temperature. That is, for the level or point, the level or the preset number of points may be calculated according to a plurality of preset grades based on the number of chats, the received evaluation results, etc.

For example, the chat temperature may increase by 0.5° C. per chat to a preset standard value (e.g., 36.5° C.), and when the received evaluation result is affirmative evaluation/satisfactory, the chat temperature may increase by 0.5° C., and when the received evaluation result is negative/not good, the chat temperature may decrease by 0.5° C.

In addition, the concept of level or points may be applied instead of the chat temperature. That is, for the level or point, the level or the preset number of points may be calculated according to a plurality of preset grades based on the number of chats, the received evaluation results, etc.

In addition, when the room leader who opens the corresponding chat room selects the preset start menu while the number of people (or number of participants) set in the chat room related to the topic talk is met, the terminal 100 selects one or more keywords the user wants to say, one or more keywords the user wants to hear, one or more additional keywords, etc., according to the user selection of the terminal 100 from the screen for selecting the keyword that the user of the terminal 100 wants to say or hear.

People with social anxiety vary depending on the situation, but when most of these people talk to others, even if it is something that they know well, they may feel anxious just from chatting with others. However, people with social anxiety feel extreme anxiety about their own ignorance, how their stories will be viewed by others, and whether what they say will be evaluated negatively by others especially when talking about topics they do not know much about. Accordingly, if anxiety about chat persists, these people may become passive in everyday chat with other people, and if it gets worse, they may become isolated and unable to socialize with people at all.

Therefore, through the function provided by the present invention, it is possible to encourage these people to actively participate in chat with anonymous people as much as possible using characters (or avatars) without their faces being exposed.

In particular, when most people chat with others about a topic, to encourage participation and leadership in chat on topics they can speak about as much as possible, such as confidently showing off their knowledge or feeling the joy of sharing knowledge when it comes to topics these people know, the present invention provides “keywords I want to say.”

In addition, by allowing people with social anxiety to choose keywords that they may say with as much confidence as possible to encourage participation and leadership in chat, the present invention provides “keywords I want to say.”

In addition, in the case of “keyword I want to hear,” it is “A topic you think is interesting, but you don't know anything about” or “A topic about which you have knowledge but are not sure about it and cannot speak confidently about it yourself.” By selecting keywords about which one would like to hear the opinions and thoughts of others rather than one's own, they can naturally participate in the chat and ultimately be encouraged to share their thoughts and knowledge with others.

In addition, the terminal 100 is linked with the server 200 and one or more other terminals (not illustrated) owned by one or more other users in the chat room related to the topic talk, to perform the chat function related to the specific topic. In this case, the terminal 100 is linked with the server 200 and uses the avatar (or character) preset in the terminal 100 to change (or apply/reflect) and display the expression (e.g., including happiness, joy, excitement, passion, pleasure, frustration, outrage, sadness, surprise, pain, rage, etc.) of the corresponding avatar according to the analysis (e.g., including a change in a user's gaze, a change in facial expression, a voice tremor, a change in heart rate, etc.) of the psychological state of the corresponding user's voice and/or facial expression while the user of the terminal 100 is chatting.

That is, the terminal 100 sequentially targets a plurality of users (e.g., including the user of the terminal 100, other users of terminals, etc.) in the chat room related to the corresponding topic talk, and performs the chat function related to the topic and keyword of the corresponding chat room once sequentially (or under the control of the room leader).

In addition, the terminal 100 performs free talking (or a free talking chat function) with a plurality of users participating in the corresponding chat room for a preset time (e.g., including 2 minutes) according to the keywords (e.g., interpersonal relationships, junior management, company life, boss, career, office worker, company, stress, other/adding keyword, etc.) set by the room leader who creates the chat room related to the topic talk.

In this way, the terminal 100 may freely perform the chat function with other users in relation to the topic, the keyword, etc., in which the user of the terminal 100 is interested.

In addition, in relation to the chat room in which the terminal 100 is participating, when the subject or keyword of the corresponding chat room is not permitted according to social norms (e.g., including drugs, human trafficking, etc.), the reporting function for the corresponding chat room may also be performed.

The embodiment of the present invention mainly describes participating in the chat room related to the topic talk that has already been generated (or opened) in the terminal 100 and performing a chat function with other users, but is not limited thereto. When a preset room creation menu is selected from the app execution result screen displayed on the terminal 100, the terminal 100 may display the room creation screen corresponding to the selected room creation menu, receive room creation information including at least one input value corresponding to at least one other input item according to the user selection (or user input/touch/control) of the corresponding terminal 100 on the displayed room creation screen, and may be linked with the server 200 based on the received room creation information to create a new chat room related to the topic talk set by the user, and linked with other terminals owned by other users participating in the created chat room and the server 200 to perform chat functions for topic talk related to a specific topic.

In addition, when the topic talk (or topic talk chat function) related to the chat room ends, the terminal 100 is linked with the server 200 to perform a self-evaluation function for the user of the corresponding terminal 100 and a counterpart evaluation function for one or more other users participating in the corresponding chat room. Here, examples of the self-evaluation function include an evaluation function for an overall satisfaction item (e.g., including disappointed, average, satisfied, other, etc.) with one's chat, an anxiety item, a confidence/assertiveness item, a mind control item, a communication skills/logic/persuasive power item, an immersion item, an empathy/listening skills item, etc. In this case, the anxiety item, the confidence/assertiveness item, the mind control item, the communication skills/logic/persuasive power item, the immersion item, the empathy/listening skills item, etc., may be selected in a preset range (e.g., 0 to 100 points) depending on the user's selection. In addition, the counterpart evaluation function includes an evaluation function for a satisfaction item (e.g., including disappointed, average, satisfied, etc.) of overall chats by different users, other user-specific chat evaluation items (e.g., including “He doesn't seem anxious at all, he's confident and assertive,” “He's good at controlling his emotions,” “He's good at communicating logically,” “He's good at immersing himself in the situation,” “He listens well and has good empathy,” etc.), etc.

That is, when the topic talk (or topic talk chat function) related to the chat room ends, the terminal 100 transmits the information related to the self-evaluation according to the user input of the terminal 100, receives the information, etc., related to counterpart evaluation of other users, and transmits the information related to the received self-evaluation, the information related to the received counterpart evaluation of other users, the identification information of the terminal 100, etc., to the server 200.

In addition, the terminal 100 receives the chat results related to the user of the corresponding terminal 100 according to the self-evaluation function and the counterpart evaluation function transmitted from the server 200 in response to information related to self-evaluation related to the previously transmitted topic talk, information related to counterpart evaluation, etc. Here, the chat result (or chat result information) includes a total average score item according to self-evaluation, an overall rating display item, an item for displaying scores (e.g., including an anxiety score, a confidence/assertiveness score, a mind control score, a communication skills/logic/persuasive power score, an immersion score, an empathy/listening skills score, etc.) for each self-evaluation item and an average score display item related to the topic talk, an evaluation item received according to the counterpart's evaluation, the number saves display item for displaying the number of times the chat content from the corresponding chat room can be saved (or downloaded), a chat recording storing item for storing the chat content, a chat time display item for displaying the total chat time in the corresponding chat room and the time the corresponding user spoke, a chat content output item for re-listening to the chat content in the corresponding chat room, etc. In addition, examples of the received evaluation item include the number of votes item received for each chat satisfaction item, the number of votes item received for each chat evaluation item, etc.

In addition, the terminal 100 displays (or outputs) the received chat results.

In addition, when a chat room related to any one role-playing is selected from one or more chat rooms related to the role-playing for the specific role (or situation) displayed in the preset chat room display area among the app execution result screen displayed on the corresponding terminal 100, the terminal 100 is linked with the server 200 to enter (or participate in) the chat room related to the selected role-playing. In this case, the terminal 100 displays the waiting screen according to the entry into the chat room. Here, the waiting screen includes the type of the corresponding chat room, the topic, the chat room name, the keyword (or hashtag), the nickname (or chat name) of the user in the corresponding chat room, the chat temperature, and the number of chats (or the number of times the user has participated in the chat room), etc. In this case, the chat temperature may be calculated based on the number of chats, the received evaluation results, etc., and the concept of level or points may be applied instead of the chat temperature. That is, for the level or points, the level or the preset number of points may be calculated according to a plurality of preset grades based on the number of chats, the received evaluation results, etc.

In addition, when the room leader who opened the corresponding chat room selects the preset start menu while the number of people (or the number of participants) set in the chat room related to the corresponding role-playing is met, the terminal 100 displays the my role description screen for confirming the description of the role of the user of the terminal 100 in the chat room related to the corresponding role-playing and the emotions felt by the user.

In addition, the terminal 100 is linked with one or more other terminals (not shown) owned by one or more other users in the chat room related to the role-playing and the server 200 to perform the chat function according to the role-playing while alternately performing the main and sub roles with the user and other users. In this case, when the chat view menu displayed on one side of the screen of the terminal 100 is selected to confirm the appropriate chat for the role while performing the chat function according to the performance of the main role or sub-role, the terminal 100 may display information (or lines/tips) on the related main role or sub-role on one side of the screen. In addition, the terminal 100 is linked with the server 200 and uses the avatar (or character) preset in the terminal 100 to change (or apply/reflect) and display the expression (e.g., including happiness, joy, excitement, passion, pleasure, frustration, outrage, sadness, surprise, pain, rage, etc.) of the corresponding avatar according to the analysis (e.g., including a change in a user's gaze, a change in facial expression, a voice tremor, a change in heart rate, etc.) of the psychological state of the corresponding user's voice and/or facial expression while the user of the terminal 100 is chatting.

That is, the terminal 100 sequentially targets a plurality of users (e.g., including the user of the terminal 100, other users of terminals, etc.) in the chat room related to the role-playing, and performs the chat function according to the main role and sub role of the corresponding chat room once sequentially (or under the control of the room leader). Here, the main role should directly answer (or deal with/respond to) to a given situation spontaneously without any set chat, and the sub-role is responsible for describing the corresponding situation description, confirming the answer of the user performing the main role, etc. In this case, the user performing the main role should answer questions on the “my role” description screen (e.g., including questions such as “How do you feel in this situation?” etc.) for confirming the description of the role and the emotion felt by the user, and the answer to this question may be used to identify the automatic thoughts in the cognitive reconstruction therapy, one of the cognitive behavior therapy programs, to evaluate the automatic thought. Here, the automatic thoughts represent (or mean) thoughts, ideas, etc., for interpreting the situation when suddenly faced with a certain situation.

In this way, the terminal 100 may freely perform the chat function with other users according to the role-playing in relation to the topic, the keyword, etc., in which the user of the terminal 100 is interested.

The embodiment of the present invention mainly describes participating in the chat room related to the role-playing that has already been generated (or opened) in the terminal 100 and performing a chat function with other users, but is not limited thereto. When a preset room creation menu is selected from the app execution result screen displayed on the terminal 100, the terminal 100 may display the room creation screen corresponding to the selected room creation menu, receive other room creation information including at least one other input value corresponding to at least one other input item according to the user selection (or user input/touch/control) of the corresponding terminal 100 on the displayed room creation screen, may be linked with the server 200 based on the received other room creation information to create a new chat room related to the role-playing set by the user, and linked with other terminals owned by other users participating in the created chat room and the server 200 to perform chat functions for role-playing related to specific roles.

In addition, when the chat room related to the role-playing is selected, after displaying the waiting screen including the type of chat room, the topic, the chat room name, the keyword, the nickname of the user entering the corresponding chat room, the chat temperature, the number of chats, etc., in the case where the start menu is selected while the number of people set in the chat room related to the role-playing is met, the embodiment of the present invention mainly describes that a “my role” description screen for confirming a description of the role of the user of the terminal 100 in the chat room related to the role-playing and the emotions felt by the user is displayed and the terminal 100 is linked with the server 200 and one or more other terminals owned by one or more other users entering the chat room related to the role-playing to perform the chat function according to the role playing while alternately performing main roles and sub roles with the user and other users, but is not limited thereto. After selecting the chat room related to the role-playing on the terminal 100 and displaying the waiting screen, the terminal 100 may be configured to set (or designate) a main role and a sub role for the user and one or more other users participating in the chat room related to the role-playing, respectively, under the control of the room leader (or other terminals owned by the room leader) who opens the chat room related to the corresponding role-playing, display a character introduction screen according to the set main role and sub role on the terminal 100 and the other terminals, respectively, display a short (or simple) situation description, and confirm the emotions of each user and then perform the chat function according to the role-playing according to the set main role and sub-role. In addition, after performing the chat function according to the role-playing according to the set main role and sub-role, the terminal 100 and the other terminals may be configured to perform the chat function according to the role-playing while alternately performing the main role and sub-role (or modifying the role) under the control of the room leader (or other terminals owned by the corresponding room leader).

In addition, when the role-playing (or role-playing chat function) related to the chat room ends, the terminal 100 is linked with the server 200 to perform a self-evaluation function for the user of the corresponding terminal 100 and a counterpart evaluation function for one or more other users participating in the corresponding chat room. Here, the self-evaluation function includes an evaluation function for overall satisfaction item (e.g., including disappointed, average, satisfied, other, etc.) with the user's chat, an anxiety item, a confidence/assertiveness item, a mind control item, a communication skills/logic/persuasive power item, an immersion item, an empathy/listening skills item, etc. In this case, the anxiety item, the confidence/assertiveness item, the mind control item, the communication skills/logic/persuasive power item, the immersion item, the empathy/listening skills item, etc., may be selected in a preset range (e.g., 0 to 100 points) depending on the user's selection. In addition, the counterpart evaluation function includes an evaluation function for a satisfaction item (e.g., including disappointed, average, satisfied, etc.) of overall chats by different users, other user-specific chat evaluation item (e.g., including “He doesn't seem anxious at all, he's confident and assertive, “He's good at controlling his emotions,” “He's good at communicating logically,” “He's good at immersing himself in the situation,” “He listens well and has good empathy,” etc.), etc.

That is, when the role-playing (or role-playing chat function) related to the chat room ends, the terminal 100 transmits the information related to the self-evaluation according to the user input of the terminal 100, receives the information, etc., related to counterpart evaluation of other users, and transmits the information related to the received self-evaluation, the information related to the received counterpart evaluation of other users, the identification information of the terminal 100, etc., to the server 200.

In addition, the terminal 100 receives other chat results related to the user according to the self-evaluation function and the counterpart evaluation function transmitted from the server 200 in response to information related to self-evaluation related to the previously transmitted role-playing, information related to counterpart evaluation, etc. Here, the other chat results (or other chat result information) include a situation description and scoring item for displaying one's initial feelings about the situation, a “my first emotion of how I felt about the situation” display item, a total average score item according to self-evaluation, an overall rating display item, an item for displaying scores (e.g., including an anxiety score, a confidence/assertiveness score, a mind control score, a communication skills/logic/persuasive power score, an immersion score, an empathy/listening skills score, etc.) for each self-evaluation item and an average score related to the corresponding role-playing, an evaluation item received according to the counterpart's evaluation, the number saves display item for displaying the number of times content from the corresponding chat room can be saved (or downloaded), a chat recording storing item for storing the corresponding chat content, a chat time display item for displaying a total chat time in the corresponding chat room and the time the user spoke, a chat content output item for re-listening to the chat content from the corresponding chat room, etc. In addition, the received evaluation item includes a number of votes item for each chat satisfaction item, a number of votes item for each chat evaluation item, etc.

In addition, the terminal 100 displays (or outputs) the other received chat results.

In addition, regarding the chat room related to the theme practice created in the terminal 100 in relation to the theme practice for a specific theme displayed in the preset chat room display area among the app execution result screen displayed on the terminal 100, when the room leader who opens the corresponding chat room selects the preset start menu while the number of people (or number of participants) set in the chat room related to the theme practice is met, the terminal 100 is linked with one or more other terminals (not shown) owned by one or more other users who have entered a chat room related to the theme practice and the server 200 to perform the chat function related to the specific theme (or topic) of the corresponding chat room. In this case, in the case where it is difficult to give an answer related to the theme while performing the chat function for the theme, when the tip menu displayed on one side of the screen of the terminal 100 is selected to check the tip, and the terminal 100 may display information (or a tip) on related themes on one side of the screen. In addition, the terminal 100 is linked with the server 200 and uses the avatar (or character) preset in the terminal 100 to change (or apply/reflect) and display the expression (e.g., including happiness, joy, excitement, passion, pleasure, frustration, outrage, sadness, surprise, pain, rage, etc.) of the corresponding avatar according to the analysis (e.g., including a change in a user's gaze, a change in facial expression, a voice tremor, a change in heart rate, etc.) of the psychological state of the corresponding user's voice and/or facial expression while the user of the terminal 100 is chatting.

That is, the terminal 100 sequentially targets a plurality of users (e.g., including the user of the terminal 100, other users of terminals, etc.) in the chat room related to the corresponding theme practice, and performs the chat function related to the theme of the corresponding chat room once sequentially (or under the control of the room leader). In this case, the terminal 100 may perform the chat function related to the corresponding theme without a spectator (or in a state without a spectator) on the chat room set as no spectator, or perform the chat function related to the corresponding theme with a spectator (or in a state having a spectator) on the chat room set as spectator according to the preset maximum number of spectators.

In this way, the terminal 100 may freely perform the chat function alone or with other users in relation to a specific theme in which the user of the terminal 100 appears to be interested.

The embodiment of the present invention mainly describes performing the chat function with other users participating in the chat room related to the theme practice generated (or opened) in the terminal 100, but is not limited thereto. When the chat room related to the theme practice is selected from one or more chat rooms related to the theme practice that has already been generated (or opened) displayed in the preset chat room display area within the app execution result screen displayed on the terminal 100, the terminal link may be linked with the server 200 to enter the chat room related to the selected theme practice and may be linked with other terminals (not illustrated) owned by other users entering the chat room related to the theme practice to perform the chat function.

In addition, when the theme practice (or theme practice chat function) related to the question-and-answer chat room ends, the terminal 100 is linked with the server 200 to perform the self-evaluation function for the user of the terminal 100. Here, the self-evaluation function includes an evaluation function for overall satisfaction item (e.g., including disappointed, average, satisfied, other, etc.) with the user's chat, an anxiety item, a confidence/assertiveness item, a mind control item, a communication skills/logic/persuasive power item, an immersion item, an empathy/listening skills item, etc. In this case, the anxiety item, the confidence/assertiveness item, the communication skills/logic/persuasive power item, the immersion item, the empathy/listening skills item, etc., may be selected in a preset range (e.g., 0 to 100 points) depending on the user's selection.

That is, when the theme practice (or theme practice chat function) related to the question-and-answer chat room ends, the terminal 100 receives the information related to the self-evaluation, etc., according to the user input of the terminal 100, and transmits the received information related to the self-evaluation, the identification information of the terminal 100, etc., to the server 200.

In addition, after the user of the terminal 100 performs the chat function through the theme practice with other users, when the theme practice (or theme practice chat function) related to the free theme chat room or the discussion theme chat room ends, the terminal 100 is linked with the server 200 to perform the self-evaluation function for the user of the terminal 100 and the counterpart evaluation function for one or more other users participating in the corresponding chat room. Here, the self-evaluation function includes an evaluation function for overall satisfaction item (e.g., including disappointed, average, satisfied, others, etc.) with the user's chat, an anxiety item, a confidence/assertiveness item, a mind control item, a communication skills/logic/persuasive power item, an immersion item, an empathy/listening skills item, etc. In this case, the anxiety item, the confidence/assertiveness item, the mind control item, the communication skills/logic/persuasive power item, the immersion item, the empathy/listening skills item, etc., may be selected in a preset range (e.g., 0 to 100 points) depending on the user's selection. In addition, the counterpart evaluation function performs a feedback (or feedback evaluation/feedback information) function on the counterpart that participates in the corresponding chat, or includes evaluation functions for chat evaluation items for each user (e.g., including “He doesn't seem anxious at all,” “He's confident and assertive, “He's good at controlling his emotions,” “He's good at communicating logically,” “He's good at immersing himself in the situation,” “He listens well and has good empathy,” etc.), etc. In this case, the terminal 100 may be configured to allow the user to directly input content into the counterpart evaluation function.

That is, when the theme practice (or theme practice chat function) related to the free theme chat room or the discussion theme chat room ends, the terminal 100 transmits the information related to the self-evaluation according to the user input of the terminal 100, receives the information, etc., related to counterpart evaluation of other users, and transmits the information related to the received self-evaluation, the information related to the received counterpart evaluation of other users, the identification information of the terminal 100, etc., to the server 200.

In addition, when the chat function is performed in the question-and-answer chat room, the terminal 100 receives the chat result related to the user of the terminal 100 according to the performance of the self-evaluation function transmitted from the server 200.

In addition, the terminal 100 displays (or outputs) the received chat results.

In addition, when the user of the terminal 100 performs the chat function through theme practice with other users (or performs the chat function in the free theme chat room or the discussion theme chat room), the terminal 100 receives another chat result related to the user of the terminal 100 according to the performance of the self-evaluation function and the counterpart evaluation function transmitted from the server 200.

In addition, the terminal 100 displays (or outputs) another received chat result.

In addition, according to the corresponding reserved counseling schedule for the expert terminal 300, when the terminal 100 is invited to the metaverse chat room (or XR counseling room/XR chat room) created by the expert terminal 300, the terminal 100 enters (or participates in) the metaverse chat room (or XR counseling room/XR chat room) created by the corresponding expert terminal 300. In this case, the terminal 100 displays the metaverse waiting screen according to the entry into the metaverse chat room. Here, the metaverse waiting screen includes the type of the corresponding metaverse chat room, the topic, the chat room name, the keyword, the nickname (or chat name) of the user in the corresponding metaverse chat room, the chat temperature, and the number of chats (or the number of times the user has participated in the chat room), etc. In this case, the chat temperature may be calculated based on the number of chats, the received evaluation results, etc., and the concept of level or points may be applied instead of the chat temperature. That is, for the level or points, the level or the preset number of points may be calculated according to a plurality of preset grades based on the number of chats, the received evaluation results, etc.

In addition, the terminal 100 is linked with the server 200 and the expert terminal 300 to receive the questionnaire transmitted (or provided) from the expert terminal 300 via the server 200. Here, the questionnaire includes one or more multiple-choice/subject questions related to a specific situation (or specific topic).

In addition, the terminal 100 displays the received questionnaire.

In addition, the terminal 100 receives questionnaire response information including a plurality of input values corresponding to the plurality of input items included in the questionnaire displayed on the terminal 100. Here, the questionnaire response information includes response information (e.g., not at all, no, average, yes, very much, etc.) for each question.

In addition, the terminal 100 transmits the received questionnaire response information, the identification information of the terminal 100, etc., to the expert terminal 300 via the server 200.

In addition, the terminal 100 receives the response results to the questionnaire related to the corresponding questionnaire response information transmitted (or provided) from the expert terminal 300 in response to the questionnaire response information related to the previously transmitted questionnaire. Here, the response results include the user's response information for each question, correct answer information for each question, and the user's earned score compared to the total points, etc.

In addition, the terminal 100 displays (or outputs) the response results to the received questionnaire.

In addition, the terminal 100 is linked with the server 200 and the expert terminal 300 to perform the psychological counseling function, the speech lesson, the mentor function, etc., with the expert through the metaverse chat room (or counseling room/XR chat room) generated from the corresponding expert terminal 300.

In addition, after the chat function ends, the terminal 200 receives the feedback information transmitted from the expert terminal 300, and displays (or outputs) the received feedback information. Here, the feedback information includes information such as an expert's view (or opinion) regarding the psychological counseling function, the speech lesson function, the mentor function, etc., performed in the metaverse chat room.

In addition, when the terminal 100 receives a request from the expert terminal 300 to participate in the metaverse chat room (or XR counseling room/XR chat room), the terminal 100 enters the metaverse chat room (or XR counseling room/XR chat room) created by the expert terminal 300 through the mediation of the server 200 or enters a specific metaverse chat room (or XR counseling room/XR chat room) selected by the expert terminal 300 from the plurality of pre-created metaverse chat rooms (or XR counseling rooms/XR chat rooms).

That is, when the terminal 100 transmits an affirmative response to the expert terminal 300 after receiving a request from the expert terminal 300 to participate in the metaverse chat room (or XR counseling room/XR chat room), the terminal 100 enters the metaverse chat room (or XR counseling room/XR chat room) created by the expert terminal 300 through the mediation of the server 200 to perform the chat function.

In addition, when the terminal 100 transmits an affirmative response to the expert terminal 300 after receiving a request from the expert terminal 300 to participate in the metaverse chat room (or XR counseling room/XR chat room), the terminal 100 enters a specific metaverse chat room (or XR counseling room/XR chat room) selected by the expert terminal 300 from among the plurality of pre-created metaverse chat rooms (or XR counseling room/XR chat room) through the mediation of the server 200.

In this case, the terminal 100 displays information indicating that the expert terminal 300 is participating in the chat as a spectator on one side of the screen of the terminal 100. Here, the terminal 100 may display icons or emoticons indicating that the expert terminal 300 is participating in the chat as a spectator on one side of the screen of the terminal 100.

In addition, the terminal 100 is linked with the expert terminal 300 owned by a specific expert selected from the plurality of located experts and the server 200 to perform the counseling function, the speech lesson function, the mentor function (or mentor-mentee function/mentoring function), etc. In this case, when the subscription function is being performed on the terminal 100, the counseling function, the speech lesson function, the mentor function, etc., with the corresponding expert may be performed free of charge, a free usage ticket assigned in advance depending on the performance of the subscription function may be deducted (or processed for use) according to the performance of the counseling function, the speech lesson function, or the mentor function, or the corresponding expert may perform and use a per-case payment function for a pre-set usage fee depending on the corresponding counseling function case, speech lesson function case, or mentor function case.

In addition, when the terminal 100 performs the counseling function, speech lesson function, mentor function, etc., with an expert, the terminal 100 may provide the personal information related to the user, the counseling record sheet (or counseling record information), the people diary (or people diary information), the chat report (or “my report” information), the psychological test results, etc., to the expert terminal 300 owned by the corresponding expert depending on the personal information use agreement in the form of an electronic document preset by the terminal 100. In this case, the digital rights management (DRM) function may be set so that the personal information related to the user provided to the expert terminal 300, the counseling record sheet (or counseling record information), the people diary (or people diary information), the chat report (or my report information), the psychological test results, etc., may not be stored, but can only be viewed.

In addition, when various types of information related to the user of the terminal 100 are provided to the expert terminal 300 or when the terminal 100 downloads the chat content according to the chat function performed by the user of the terminal 100 in the form of a file, the server 200 manages (or stores) log records in which the corresponding information is provided, log records in which the corresponding chat content is downloaded, etc. Here, the log record includes the type of information/chat content provided, date and time information on when it was provided/downloaded, information on the user/terminal that received/downloaded it, etc.

In this way, the terminal 100 may perform a topic talk chat room function, a role-playing chat room function, a theme practice chat room function, a people diary writing and/or management function, a “my report” management function, etc.

The embodiment of the present invention describes that the terminal 100 performs a chat room search function, a chat function/user-to-user chat function, a counseling with expert function/expert search function, a room creation function, a topic talk chat room function, a role-playing chat room function, a theme practice chat room function, a people diary writing and/or management function, a “my report” management function, etc., in the form of the dedicated app, but is not limited thereto, and it may perform the chat room search function, the chat function/user-to-user chat function, the counseling with expert function/expert search function, the room creation function, the topic talk chat room function, the role-playing chat room function, the theme practice chat room function, the people diary writing and/or management function, the “my report” management function, etc., through the website provided on the server 200, etc., in addition to the dedicated app.

The server 200 communicates with the terminal 100, the expert terminal 300, etc.

In addition, the server 200 performs a member subscription process for a user of the terminal 100, the expert terminal 300, etc.

In addition, the server 200 registers personal information related to the user of the terminal 100, the expert terminal 300, etc. In this case, the server 200 registers (or manages) the corresponding personal information, etc., in a DB server (not shown), and may be provided with a personal information use consent form in advance in the form of an electronic document from the user of the corresponding terminal 100, the expert of the expert terminal 300, etc.

In addition, the server 200 performs a member management function for a user of the terminal 100, the expert terminal 300, etc.

In this case, the server 200 provides the terminal 100, the expert terminal 300, etc., with the dedicated app and/or website providing a chat room search function, a chat function/user-to-user chat function, a counseling with expert function/expert search function, a room creation function, a topic talk chat room function, a role-playing chat room function, a theme practice chat room function, a people diary writing and/or management function, a “my report” management function, a counseling function for a user, a people diary/“my report” check function for a specific user while performing the counseling function, a counseling result writing and management function, a speech lesson function, a mentor function (or mentoring function), a psychological counseling function, an XR chat room (or metaverse chat room) creation and management function, etc. In this case, the dedicated app may be configured as different user interfaces (UIs) according to a user who receives a chat room search function, a chat function/user-to-user chat function, a counseling with expert function/expert search function, a room creation function, a topic talk chat room function, a role-playing chat room function, a theme practice chat room function, a people diary writing and/or management function, a “my report” management function, etc., and an expert (or counselor) who receives a counseling function for a user, a people diary/“my report” check function for a specific user while performing the counseling function, a counseling result writing and management function, a speech lesson function, a mentor function (or mentoring function), a psychological counseling function, an XR chat room (or metaverse chat room) creation and management function, etc.

In addition, the server 200 provides a bulletin board function for notices, events, etc.

In addition, when the terminal 100 sets up (or requests) the subscription function to perform the chat room function, the counseling function, etc. provided by the server 200, the server 200 is linked with the terminal 100 and the payment server to perform a payment function for subscription fees (or costs) related to the corresponding chat room function, the counseling function, etc. In this case, the server 200 may perform card payment, automatic transfer function through linkage with a bank payment account, payment using cash points or cash remaining in the account of the terminal 100 that is subscribed as a member of the server 200, and a payment function through simple payments including Kakao Pay, Naver Pay, etc.

When the payment function fails, the server 200 provides the payment failure information (e.g., including payment date, payment amount, failure information (e.g. insufficient balance, limit exceeded, etc.), etc.) (or information indicating that the payment has failed) to the terminal 100.

In addition, when the payment function is performed normally, the server 200 provides the payment function performance result to the terminal 100. Here, the result of performing the payment function includes the subscription period, the payment amount information, the payment date and time information, etc.

In addition, according to the performance of the subscription function in the terminal 100, when the terminal 100 wants to participate in the chat room and perform the chat function or the counseling function with an expert, the server 200 provides the chat function through the linkage between the corresponding terminal 100 and other terminals, or provides the counseling function through the linkage between the corresponding terminal 100 and the expert terminal 300. In this case, when performing the chat function, the counseling function, etc., on the terminal 100, the server 200 may perform the payment function according to a preset usage fee for each case, and after the payment function ends normally, provide the corresponding terminal 100 with the chat function, the counseling function, etc., related to the corresponding payment case.

In addition, the server 200 receives the search word, the identification information of the terminal 100, etc., transmitted from the terminal 100.

In addition, the server 200 searches for (or confirms) the chat room corresponding to the search word among one or more chat rooms opened in the server 200.

As the search result (or the confirmation result), when there is no chat room corresponding to the search word among one or more chat rooms opened in the corresponding server 200, the server 200 transmits the information indicating that the chat room corresponding to the corresponding search word does not exist (or information indicating that the chat room registered corresponding to the corresponding search word does not exist) to the terminal 100.

In addition, as the search result (or the confirmation result), when there is a chat room corresponding to the search word among one or more chat rooms opened in the corresponding server 200, the server 200 transmits the list of chat rooms corresponding to the search word to the terminal 100. Here, the list of chat rooms includes the number of chat rooms found, a type of chat room, a topic, a chat room name, a keyword, a nickname (or screen name) of a user in the corresponding chat room, the number of people currently inside compared to the total number of people, whether the chat is in progress (e.g., waiting, chatting, chat over, etc.), an entry menu (or no entry menu), etc.

In addition, the server 200 receives the expert search information, the identification information of the terminal 100, etc., transmitted from the terminal 100.

In addition, the server 200 searches for (or confirms) an expert corresponding to the expert search information among one or more experts registered in the server 200.

As the search results (or the confirmation results), when the expert corresponding to the expert search information is not found among one or more experts registered in the server 200, the server 200 transmits information indicating that the expert corresponding to the corresponding expert search information has not yet been found to the terminal 100.

In addition, As the search results (or the confirmation results), when the expert corresponding to the expert search information is found among one or more experts registered in the server 200, the server 200 transmits one or more pieces of expert information corresponding to the found expert search information to the terminal 100. Here, the expert information includes an expert name, a counseling field (or specialty field) (e.g., psychological counseling, speech lesson, mentoring, etc.), a brief introduction, a photo (or image), a fee per session, a rating (or star rating), review information, immediate counseling availability, popularity (or number of likes), etc.

In addition, when the specific expert information is selected from information on one or more experts displayed on the terminal 100, the server 200 receives specific expert information, detailed expert information request information, identification information of the terminal 100, etc., transmitted from the terminal 100.

In addition, the server 200 confirms (or searches for) the expert detailed information corresponding to the received specific expert information among the expert detailed information for each expert registered in the server 200.

In addition, the server 200 transmits detailed expert information corresponding to the confirmed (or found) specific expert information to the terminal 100. Here, the detailed expert information includes an expert name item, a counseling field (or specialty) item (e.g., including psychological counseling, speech lesson, mentoring, etc.), a rating (or star rating) item, a photo (or image) item, a brief introduction item, a usage fee per counseling session, a re-use desire rate of users who received counseling item, a total number of counseling sessions item, a counseling information item, a counselor information item, a review item, etc. In addition, the counseling information item includes a price option item (e.g., including a basic counseling item, a severe anxiety counseling item, etc.), a detailed description item, a location item, a popularity (or number of likes) item, a counseling application item, etc. In addition, the basic counseling item, the severe anxiety counseling item, etc., include the number of counseling sessions, additional composition (e.g., XR chat room training 40%, data analysis 60%, etc.), time (e.g., including 1 hour, etc.), price (e.g., 50,000 won/time), etc. In addition, the counselor information item includes a counselor introduction, a career introduction, a certification introduction, etc. In addition, the review item includes an overall star rating, ratings for each category (e.g., including use of XR, friendliness, analytical skills, empathy and comfort, appropriate advice, etc.), the total review, etc.

In addition, the server 200 performs a counseling questionnaire writing function for the user of the terminal 100 to whom a new member subscribes.

In addition, the server 200 is linked with the terminal 100 to map (or match/link) the detailed emotional record information with the user information of the terminal 100 and stores the mapped information in the corresponding server 200 (or registration/management).

That is, the server 200 receives the detailed emotional record information, the emotional record storage request information, the identification information of the terminal 100, etc., transmitted from the terminal 100.

In addition, the server 200 confirms (or searches for) the user information corresponding to the received identification information of the terminal 100 among user information (or personal information/member information/expert information) for each piece of identification information of the terminal being managed by the server 200.

In addition, the server 200 maps (or matches/links) the user information corresponding to the identified (or searched) identification information of the corresponding terminal 100 to the received detailed emotional record information, the received identification information of the terminal 100, etc., and stores (or registers/manages) the mapped information in the corresponding server 200.

In addition, the server 200 is linked with the terminal 100 to map (or match/link) the simple emotional record information with the user information of the terminal 100 and stores the mapped information in the corresponding server 200 (or registration/management).

That is, the server 200 receives the simple emotional record information, the other emotional record storage request information, the identification information of the terminal 100, etc., transmitted from the terminal 100.

In addition, the server 200 confirms (or searches for) the user information corresponding to the received identification information of the terminal 100 among user information (or personal information/member information) for each piece of identification information of the terminal being managed by the server 200.

In addition, the server 200 maps (or matches/links) the user information corresponding to the identified (or searched) identification information of the corresponding terminal 100 to the received simple emotional record information, the received identification information of the terminal 100, etc., and stores (or registers/manages) the mapped information in the corresponding server 200.

In addition, when a preset detailed score view item is selected from the detailed information included in the content display area displayed on the terminal 100, the terminal 100 is linked with the server 200 to display detailed score information corresponding to the selected detailed score view item. Here, the detailed score information (or detailed score screen) includes the sort items for sorting functions related to all, topic talk, role-playing, theme practice, etc., the “number of times you've participated in the chat room” display item, the period display item for displaying the corresponding preset period, the “number of times you've participated in the chat room for each category” display item, the “free expression” index display item, the anxiety index display item, the relative evaluation display item, the “my ‘free expression’ score aspect” display item, the “my anxiety score aspect” display item, the “my confidence/assertiveness, mind control, communication skills/logic/persuasive power, immersion, empathy/listening skills score pattern” display item, the “topic I have practiced a lot” display item, the “keyword that I have practiced a lot” display item, the “target that I have practiced a lot” display item, the “situation that I have practiced a lot” display item, the “Which emotions I am sensitive to?” display item, the “Which experts have I practiced with a lot?” display item, the “theme which I have practiced a lot” display item, etc.

In addition, when the participation list item in the preset user-to-user item is selected from the “my report” screen displayed on the terminal 100, to provide the participation list related to the chat list in which the user of the terminal 100 participates, the server 200 is linked with the terminal 100 to provide the participation list screen corresponding to the participation list item to the terminal 1000 Here, the participation list screen includes a sort item for sorting functions related to all, topic talk, role-playing, theme practice, etc., a change information display item for providing information on changes in self-evaluation results or other's evaluation results compared to a preset period (or last week), a “number of times you've participated in the chat room” display item for displaying the number of times the user has participated in the chat room within a preset period, an information display item for each chat participation case for providing information related to topic talk, role-playing, theme practice, etc., in which the user participates, etc. In this case, the information display item for each chat participation includes a date information display item of participation in the chat room, a detail view item for displaying the detailed information, a chat room type (or chat room category) display item (e.g., including topic talk, role-playing, theme practice, etc.) for displaying the type of chat room in which the user participates, a chat room topic display item for displaying the topic of the chat room, a chat room name display item for displaying the room title (or chat room name) of the chat room, a keyword display item for displaying the main keywords/hashtags related to the corresponding chat room, a self-evaluation information display item for displaying the self-evaluation results, a received evaluation information display item for displaying the evaluation information received from other users participating in the corresponding chat room, a chat recording display item for the re-listening function of chat content from the chat room, etc.

In addition, when a specific participation list (or a detail view item displayed on one side of the corresponding specific participation list) is selected from one or more participation lists displayed in the information display items for each chat participation in the participation list screen displayed on the terminal 100, the server 200 provides the detail view information related to the selected specific participation list to the terminal 100. Here, the detailed view information includes a date information display item of participation in the chat room corresponding to the selected specific participation list, a chat room type (or chat room category) display item for displaying the type of chat room in which the user participates (e.g., including a topic talk chat room, a role-playing chat room, a theme practice chat room, etc.), a chat room topic display item for displaying the topic of the chat room, a chat room name display item for displaying the room title of the chat room, a keyword display item for displaying main keywords/hashtags related to the chat room, a chat participant information display item for displaying information (e.g., including nicknames) of other users participating in the corresponding chat room, a self-evaluation result display item (or self-evaluation information display item) for displaying self-evaluation results, a received evaluation result display item (or a received evaluation information display item) for displaying evaluation results received from other users participating in the chat room, a chat recording display item for re-listening to chat content from the chat room, etc.

In addition, when the cognitive behavior therapy record item in the preset expert interaction item is selected from the “my report” screen displayed on the terminal 100, to provide expert feedback according to the user of the terminal 100 performing a training function on an appropriate chat room type in relation to a specific topic or situation, the server 200 provides the cognitive behavior therapy record screen corresponding to the cognitive behavior therapy record item to the terminal 100.

In addition, when the writing and modification item displayed on one side of the specific cognitive behavior therapy record is selected from among one or more cognitive behavior therapy record items displayed in the cognitive behavior therapy record information display item for each chat participation in the cognitive behavior therapy record screen displayed on the terminal 100, the server 200 provides the cognitive behavior therapy record input screen corresponding to the selected writing and modification item to the terminal 100 to edit the cognitive behavior therapy record information related to the selected specific cognitive behavior therapy record. Here, in the case of the pre-training, the cognitive behavior therapy record input screen includes A “What are situations that make you anxious?” item, A “How are you feeling at this time” item (e.g., including anger/fury, irritation, disbelief, discomfort, disappointment, sadness/depression, anxiety, tiredness, fatigue, frustration, bitterness, remorse, jealousy, shame, embarrassment, despondence, outrage, panic, isolation, loneliness, boredom, etc.), a “What are your thoughts in that situation?” item (e.g., including a confidence level, a cognitive error, etc.), a “Please correct my wrong thoughts” item (or reasonable response finding item), a behavioral goal setting item, etc. In this case, the types of cognitive errors include black and white (or black and white logic), divination (or fortune teller/catastrophe), ignoring (or ignoring advantages), inference (or emotional inference), name (or naming), mind reading (or mind reading technique), filtering (or mental filtering), obsession (or obsessive burden), uselessness (or useless thoughts), etc. In addition, in the case of the post-training, the cognitive behavior therapy record input screen includes a “Please display any area that has improved slightly through training” item (e.g., improvement-related item, anxiety-related item, etc.), a “What actually happened related to an area I was worried about before training?” item, a “Please re-evaluate your thoughts before training” item, a “how I feel now” selection item (e.g. happiness, joy, vitality, confidence, courage, spirit, gratitude, appreciation, passion, inspiration, ecstasy, love, anger/rage, irritation, disbelief, displeasure, disappointment, sadness/depression, anxiety, tiredness, fatigue, frustration, etc.), a “What did you learn?” item, etc.

In addition, the server 200 is linked with the terminal 100 to apply the plurality of input values to the user information of the terminal 100 and the cognitive behavior therapy record information related to the specific cognitive behavior therapy record case and provide the terminal 100 with the cognitive behavior therapy record screen in the state in which the plurality of input values are applied (or updated).

In addition, when the item for writing a new cognitive behavior therapy record sheet in the cognitive behavior therapy record screen displayed on the terminal 100 is selected, the server 200 is linked with the terminal 100 to provide the terminal 100 with a new cognitive behavior therapy record sheet writing screen corresponding to the new cognitive behavior therapy record sheet writing item. Here, the new cognitive behavior therapy record sheet creation screen includes a cognitive behavior therapy record sheet title input item for inputting the cognitive behavior therapy record sheet title, a pre-training or post-training item for selecting pre/post-training, a pre-training input item (e.g., including “What are situations that make you anxious?” “How are you feeling at this time?” “What are your thoughts in that situation?” “Please correct my wrong thoughts,” “Please set action goal,” etc.), a post-training input item (e.g., including “Please display any area that has improved slightly through training,” “What actually happened in relation to an area I was worried about before training?” “Please re-evaluate your thoughts before training,” “How do you feel now, and what have you learned?” etc.) etc.

In addition, the server 200 is linked with the terminal 100 to generate the a new cognitive behavior therapy record related to the user (or user information) of the terminal 100 based on the plurality of received different input values, apply the plurality of different input values to the cognitive behavior therapy record information related to the generated new cognitive behavior therapy record case, and provide the terminal 100 with the cognitive behavior therapy record screen in the state in which the plurality of different input values are applied.

In addition, when a chat room related to any one topic talk is selected from one or more chat rooms related to the topic talk displayed in the preset chat room display area among the app execution result screen displayed on the corresponding terminal 100, the server 200 is linked with the terminal 100 to cause the terminal 100 to enter (or participate in) the chat room related to the selected topic talk. In this case, the server 200 provides the terminal 100 with a waiting screen according to the entry of the corresponding terminal 100 into the chat room. Here, the waiting screen includes the type of the corresponding chat room, the topic, the chat room name, the keyword, the nickname (or chat name) of the user in the corresponding chat room, the chat temperature, and the number of chats (or the number of times the user has participated in the chat room), etc. In this case, the chat temperature may be calculated based on the number of chats, the received evaluation results, etc., and the concept of level or points may be applied instead of the chat temperature. That is, for the level or points, the level or a preset number of points may be calculated according to a plurality of preset grades based on the number of chats, the received evaluation results, etc.

In addition, when the number of people set in the chat room related to the corresponding topic talk is satisfied, the server 200 is linked with the terminal 100 participating in the chat room related to the corresponding topic talk and one or more other terminals (not illustrated) each owned by one or more other users to perform (or manage/control) the chat function according to the corresponding topic talk. In this case, the server 200 automatically stores the chat content of the user of the terminal 100 and other users of the terminal according to the performance of the chat function.

In addition, the server 200 analyzes the content spoken by the user of the terminal 100 and other users of terminals, and changes (or applies/reflects) and provides the avatar (or character) related to the user of the terminal 100 and the facial expression (e.g., including happiness, joy, excitement, passion, pleasure, frustration, outrage, sadness, surprise, pain, rage, etc.) of the avatar related to the other users of terminals according to the psychological state analysis (e.g., including a change in a user's gaze, a change in facial expression, a voice tremor, a change in heart rate, etc.).

In addition, when the topic talk (or topic talk chat function) related to the chat room ends, the server 200 is linked with the terminal 100 to perform a self-evaluation function for the user of the corresponding terminal 100 and a counterpart evaluation function for one or more other users participating in the corresponding chat room. Here, the self-evaluation function includes an evaluation function for overall satisfaction item (e.g., including disappointed, average, satisfied, other, etc.) with “my chat,” an anxiety item, a confidence/assertiveness item, a mind control item, a communication skills/logic/persuasive power item, an immersion item, an empathy/listening skills item, etc. In this case, the anxiety item, the confidence/assertiveness item, the mind control item, the communication skills/logic/persuasive power item, the immersion item, the empathy/listening skills item, etc., may be selected in a preset range (e.g., 0 to 100 points) depending on the user's selection. In addition, the counterpart evaluation function includes an evaluation function for a satisfaction item (e.g., including disappointed, average, satisfied, etc.) of overall chats by different users, other user-specific chat evaluation items (e.g., including “He doesn't seem anxious at all,” “He's confident and assertive,” “He's good at controlling his emotions,” “He's good at communicating logically,” “He's good at immersing in the situation,” “He listens well and has good empathy,” etc.), etc.

That is, when the topic talk (or topic talk chat function) related to the chat room ends, the server 200 receives (or collects) the information related to the self-evaluation from the plurality of terminals 100 participating in the chat room related to the corresponding topic talk, the information related to the counterpart evaluations by other users, the identification information of the terminal 100, etc.

In addition, the server 200 generates chat results for each user participating in the corresponding chat room according to the performance of the self-evaluation function and the counterpart evaluation function performed by each of the plurality of terminals participating in the topic talk related to the corresponding chat room. Here, the chat result (or chat result information) includes a total average score item according to self-evaluation, an overall rating display item, an item for displaying scores (e.g., including an anxiety score, a confidence/assertiveness score, a mind control score, a communication skills/logic/persuasive power score, an immersion score, an empathy/listening skills score, etc.) for each self-evaluation item and an average score display item related to the topic talk, an evaluation item received according to the counterpart's evaluation, the number saves display item for displaying the number of times the chat content from the corresponding chat room can be saved (or downloaded), a chat recording storing item for storing the chat content, a chat time display item for displaying the total chat time in the corresponding chat room and the time the corresponding user spoke, a chat content output item for re-listening to the chat content in the corresponding chat room, etc. In addition, examples of the received evaluation item include the number of votes item received for each chat satisfaction item, the number of votes item received for each chat evaluation item, etc.

That is, the server 200 generates different chat results for each user participating in the corresponding chat room based on the information related to the self-evaluation received (or collected) from each of the plurality of terminals 100 participating in the chat room related to the corresponding topic talk, the information related to the counterpart evaluation of other users, etc.

In addition, the server 200 transmits the generated chat results for each user to the terminal 100 owned by each user.

In addition, the server 200 updates (or calculates/computes) the free expression index, the anxiety index, the chat temperature (or level/point), etc., related to the user based on another chat results generated in relation to the user of the terminal 100.

In addition, when a chat room related to any one role-playing is selected from one or more chat rooms related to the role-playing for the specific role (or situation) displayed in the preset chat room display area within the app execution result screen displayed on the corresponding terminal 100, the server 200 is linked with the terminal 100 to cause the corresponding terminal 100 to enter (or participate in) the chat room related to the selected role-playing. In this case, the server 200 provides the terminal 100 with a waiting screen according to the entry of the corresponding terminal 100 into the chat room. Here, the waiting screen includes the type of the corresponding chat room, the topic, the chat room name, the keyword (or hashtag), the nickname (or chat name) of the user in the corresponding chat room, the chat temperature, and the number of chats (or the number of times the user has participated in the chat room), etc. In this case, the chat temperature may be calculated based on the number of chats, the received evaluation results, etc., and the concept of level or points may be applied instead of the chat temperature. That is, for the level or points, the level or the preset number of points may be calculated according to a plurality of preset grades based on the number of chats, the received evaluation results, etc.

In addition, when the number of people set in the chat room related to the role-playing is satisfied, the server 200 is linked with the terminal 100 participating in the chat room related to the role-playing and one or more other terminals (not illustrated) each owned by one or more other users to perform (or manage/control) the chat function according to the role-playing while alternately performing main and sub roles with the user and other users. In this case, the server 200 automatically stores the chat content of the user of the terminal 100 and other users of the terminal according to the performance of the chat function.

In addition, the server 200 analyzes the content spoken by the user of the terminal 100 and other users of terminals, and changes (or applies/reflects) and provides the avatar (or character) related to the user of the terminal 100 and the facial expression (e.g., including happiness, joy, excitement, passion, pleasure, frustration, outrage, sadness, surprise, pain, rage, etc.) of the avatar related to the other users of terminals according to the psychological state analysis (e.g., including a change in a user's gaze, a change in facial expression, a voice tremor, a change in heart rate, etc.).

In addition, when the role-playing (or role-playing chat function) related to the chat room ends, the server 200 is linked with the terminal 100 to perform a self-evaluation function for the user of the corresponding terminal 100 and a counterpart evaluation function for one or more other users participating in the corresponding chat room. Here, the self-evaluation function includes an evaluation function for overall satisfaction item (e.g., including disappointed, average, satisfied, other, etc.) with the user's chat, an anxiety item, a confidence/assertiveness item, a mind control item, a communication skills/logic/persuasive power item, an immersion item, an empathy/listening skills item, etc. In this case, the anxiety item, the confidence/assertiveness item, the mind control item, the communication skills/logic/persuasive power item, the immersion item, the empathy/listening skills item, etc., may be selected in a preset range (e.g., 0 to 100 points) depending on the user's selection. In addition, the counterpart evaluation function includes an evaluation function for a satisfaction item (e.g., including disappointed, average, satisfied, etc.) of overall chats by different users, other user-specific chat evaluation item (e.g., including “He doesn't seem anxious at all, he's confident and assertive, “He's good at controlling his emotions,” “He's good at communicating logically,” “He's good at immersing himself in the situation,” “He listens well and has good empathy,” etc.), etc.

That is, when the role-playing (or role-playing chat function) related to the chat room ends, the server 200 receives (or collects) the information related to the self-evaluation from the plurality of terminals 100 participating in the chat room related to the corresponding role-playing, the information related to the counterparty evaluations by other users, the identification information of the terminal 100, etc.

In addition, the server 200 generates other chat results for each user participating in the corresponding chat room according to the performance of the self-evaluation function and the counterpart evaluation function performed by each of the plurality of other terminals participating the role-playing related to the corresponding chat room. Here, the other chat results (or other chat result information) include a situation description and scoring item for displaying one's initial feelings about the situation, a “my first emotion of how I felt about the situation” display item, a total average score item according to self-evaluation, an overall rating display item, an item for displaying scores (e.g., including an anxiety score, a confidence/assertiveness score, a mind control score, a communication skills/logic/persuasive power score, an immersion score, an empathy/listening skills score, etc.) for each self-evaluation item and an average score related to the corresponding role-playing, an evaluation item received according to the counterpart's evaluation, the number saves display item for displaying the number of times content from the corresponding chat room can be saved (or downloaded), a chat recording storing item for storing the corresponding chat content, a chat time display item for displaying a total chat time in the corresponding chat room and the time the user spoke, a chat content output item for re-listening to the chat content from the corresponding chat room, etc. In addition, the received evaluation item includes a number of votes item for each chat satisfaction item, a number of votes item for each chat evaluation item, etc.

That is, the server 200 generates different chat results for each user participating in the corresponding chat room based on the information related to the self-evaluation received (or collected) from each of the plurality of terminals 100 participating in the chat room related to the corresponding role-playing, the information related to the counterpart evaluation of other users, etc.

In addition, the server 200 transmits the generated other chat results for each user to the terminal 100 owned by each user.

In addition, the server 200 updates (or calculates/computes) the free expression index, the anxiety index, the chat temperature (or level/point), etc., related to the user based on other chat results generated in relation to the user of the terminal 100.

In addition, when a preset room creation menu is selected from the app execution result screen displayed on the terminal 100, to create a new chat room related to the category the user wants, the server 200 provides (or transmits) a room creation screen corresponding to the selected room creation menu to the terminal 100. Here, the room creation screen includes a room type selection menu for selecting a room type, etc. In addition, the room type selection menu includes the topic talk item for free chat between users with similar interests, a role-playing item for practicing expressing emotions and opinions by setting up situations, a theme practice item for developing (or practicing) adaptability and appropriate responsiveness by creating the theme that may cause tension, etc.

In addition, when a preset theme practice item is selected from the room creation screen displayed on the corresponding terminal 100, to develop (or practice) adaptability and appropriate responsiveness by creating a theme that may cause tension, the server 200 provides (or transmits) a spectator type setting screen corresponding to the selected theme practice item to the terminal 100.

In addition, when a preset no spectator item is selected from the spectator type setting screen displayed on the terminal 100, to create a theme practice chat room for performing the chat function with the question-and-answer, the server 200 provides (or transmits) a theme setting screen corresponding to the selected no spectator item to the terminal 100.

In addition, when a preset question-and-answer item is selected from the theme setting screens displayed on the terminal 100, the server 200 provides (or transmits) a questionnaire setting screen for receiving self-introduction or question content to the terminal 100.

In addition, after receiving the questionnaire setting information from the questionnaire setting screen, when the registration item displayed on one side of the questionnaire setting screen is selected, the server 200 provides (or transmits) a detailed theme setting screen to which the questionnaire setting information is applied (or reflected) to the terminal 100.

In addition, after receiving the detailed theme setting information on the detailed theme setting screen displayed on the terminal 100, when the room creation item (or creation item) displayed on one side of the detailed theme setting screen is selected, the server 200 is linked with the terminal 100 to create (or open) a new theme practice chat room (or question-and-answer theme practice chat room) corresponding to the detailed theme setting information. In this case, the theme practice chat room creation may be performed in the terminal 100 that is performing a subscription function in advance or in the terminal 100 that has paid a preset usage amount for each topic to create the theme practice chat room. In addition, the server 200 provides (or transmits) a waiting screen to the terminal 100 according to the entry of the terminal 100 into the chat room. Here, the waiting screen includes the type of the corresponding chat room, the topic, the chat room name, the keyword, the nickname (or chat name) of the user who enters the corresponding chat room, the chat temperature, and the number of chats (or the number of times the user has participated in the chat room), etc. In this case, the chat temperature may be calculated based on the number of chats, the received evaluation results, etc., and the concept of level or points may be applied instead of the chat temperature. That is, for the level or point, the level or the preset number of points may be calculated according to a plurality of preset grades based on the number of chats, the received evaluation results, etc.

In addition, when a preset presence spectator item is selected from the spectator type setting screen displayed on the terminal 100, to create a theme practice chat room in the form of a free theme or discussion theme, the server 200 provides (or transmits) other theme setting screens corresponding to the selected presence spectator item to the terminal 100.

In addition, when any one theme item is selected from other theme setting screens displayed on the terminal 100, the server 200 provides (or transmits) other detailed theme setting screens for receiving the room title, the number of participants in training, or the maximum number of spectators to the terminal 100.

In addition, after receiving other theme detailed setting information on other theme detailed setting screens displayed on the terminal 100, when the room creation item (or creation item) displayed on one side of other theme detailed setting screens is selected, the server 200 is linked with the terminal 100 to create (or open) a new theme practice chat room (or free theme practice chat room/discussion theme practice chat room) corresponding to the other detailed theme setting information. In this case, the theme practice chat room creation may be performed in the terminal 100 that is performing a subscription function in advance or in the terminal 100 that has paid a preset usage amount for each topic to create the theme practice chat room. In addition, the server 200 provides (or transmits) a waiting screen to the terminal 100 according to the entry of the terminal 100 into the chat room. Here, the waiting screen includes the type of the corresponding chat room, the topic, the chat room name, the keyword, the nickname (or chat name) of the user who enters the corresponding chat room, the chat temperature, and the number of chats (or the number of times the user has participated in the chat room), etc. In this case, the chat temperature may be calculated based on the number of chats, the received evaluation results, etc., and the concept of level or points may be applied instead of the chat temperature. That is, for the level or point, the level or the preset number of points may be calculated according to a plurality of preset grades based on the number of chats, the received evaluation results, etc.

In addition, when the room leader who opens the corresponding chat room selects the preset start menu while the number of people (or the number of participants) set in the chat room related to the corresponding theme practice is met, the terminal 100 is linked with one or more other terminals 100 (not illustrated) owned by one or more other users in the corresponding chat room related to the corresponding theme practice and the server 200 to perform the chat function related to the specific theme (or topic) of the corresponding chat room. In this case, in the case where it is difficult to give an answer related to the theme while performing the chat function for the theme, when the tip menu displayed on one side of the screen of the terminal 100 is selected to check the tip, the terminal 100 may display information (or a tip) on related themes on one side of the screen. In addition, the terminal 100 is linked with the server 200 and uses the avatar (or character) preset in the terminal 100 to change (or apply/reflect) and display the expression (e.g., including happiness, joy, excitement, passion, pleasure, frustration, outrage, sadness, surprise, pain, rage, etc.) of the corresponding avatar according to the analysis (e.g., including a change in a user's gaze, a change in facial expression, a voice tremor, a change in heart rate, etc.) of the psychological state of the corresponding user's voice and/or facial expression while the user of the terminal 100 is chatting.

In addition, when the theme practice (or theme practice chat function) related to the question-and-answer chat room ends, the server 200 is linked with the terminal 100 to perform the self-evaluation function for the user of the terminal 100. Here, the self-evaluation function includes an evaluation function for overall satisfaction item (e.g., including disappointed, average, satisfied, other, etc.) with the user's chat, an anxiety item, a confidence/assertiveness item, a mind control item, a communication skills/logic/persuasive power item, an immersion item, an empathy/listening skills item, etc. In this case, the anxiety item, the confidence/assertiveness item, the communication skills/logic/persuasive power item, the immersion item, the empathy/listening skills item, etc., may be selected in a preset range (e.g., 0 to 100 points) depending on the user's selection.

That is, when the theme practice (or theme practice chat function) related to the question-and-answer chat room ends, the server 200 receives (or collects) the information related to the self-evaluation, the identification information of the terminal 100, etc., from the terminal 100 that participates in the question-and-answer chat room related to the corresponding theme practice and performs the chat function. In addition, when a preset room creation menu is selected from the app execution result screen displayed on the terminal 100, to create a new chat room related to the category the user wants, the server 200 provides (or transmits) a room creation screen corresponding to the selected room creation menu to the terminal 100. Here, the room creation screen includes a room type selection menu for selecting a room type, etc. In addition, the room type selection menu includes the topic talk item for free chat between users with similar interests, a role-playing item for practicing expressing emotions and opinions by setting up situations, a theme practice item for developing (or practicing) adaptability and appropriate responsiveness by creating the theme that may cause tension, etc.

In addition, when a preset theme practice item is selected from the room creation screen displayed on the corresponding terminal 100, to develop (or practice) adaptability and appropriate responsiveness by creating a theme that may cause tension, the server 200 provides (or transmits) a spectator type setting screen corresponding to the selected theme practice item to the terminal 100.

In addition, when a preset no spectator item is selected from the spectator type setting screen displayed on the terminal 100, to create a theme practice chat room for performing the chat function with the question-and-answer, the server 200 provides (or transmits) a theme setting screen corresponding to the selected no spectator item to the terminal 100.

In addition, when a preset question-and-answer item is selected from the theme setting screens displayed on the terminal 100, the server 200 provides (or transmits) a questionnaire setting screen for receiving self-introduction or question content to the terminal 100.

In addition, after receiving the questionnaire setting information from the questionnaire setting screen, when the registration item displayed on one side of the questionnaire setting screen is selected, the server 200 provides (or transmits) a detailed theme setting screen to which the questionnaire setting information is applied (or reflected) to the terminal 100.

In addition, after receiving the detailed theme setting information on the detailed theme setting screen displayed on the terminal 100, when the room creation item (or creation item) displayed on one side of the detailed theme setting screen is selected, the server 200 is linked with the terminal 100 to create (or open) a new theme practice chat room (or question-and-answer theme practice chat room) corresponding to the detailed theme setting information. In this case, the theme practice chat room creation may be performed in the terminal 100 that is performing a subscription function in advance or in the terminal 100 that has paid a preset usage amount for each topic to create the theme practice chat room. In addition, the server 200 provides (or transmits) a waiting screen to the terminal 100 according to the entry of the terminal 100 into the chat room. Here, the waiting screen includes the type of the corresponding chat room, the topic, the chat room name, the keyword, the nickname (or chat name) of the user who enters the corresponding chat room, the chat temperature, and the number of chats (or the number of times the user has participated in the chat room), etc. In this case, the chat temperature may be calculated based on the number of chats, the received evaluation results, etc., and the concept of level or points may be applied instead of the chat temperature. That is, for the level or point, the level or the preset number of points may be calculated according to a plurality of preset grades based on the number of chats, the received evaluation results, etc.

In addition, when a preset presence spectator item is selected from the spectator type setting screen displayed on the terminal 100, to create a theme practice chat room in the form of a free theme or discussion theme, the server 200 provides (or transmits) other theme setting screens corresponding to the selected presence spectator item to the terminal 100.

In addition, when any one theme item is selected from other theme setting screens displayed on the terminal 100, the server 200 provides (or transmits) other detailed theme setting screens for receiving the room title, the number of participants in training, or the maximum number of spectators to the terminal 100.

In addition, after receiving other theme detailed setting information on other theme detailed setting screens displayed on the terminal 100, when the room creation item (or creation item) displayed on one side of other theme detailed setting screens is selected, the server 200 is linked with the terminal 100 to create (or open) a new theme practice chat room (or free theme practice chat room/discussion theme practice chat room) corresponding to the other detailed theme setting information. In this case, the theme practice chat room creation may be performed in the terminal 100 that is performing a subscription function in advance or in the terminal 100 that has paid a preset usage amount for each topic to create the theme practice chat room. In addition, the server 200 provides (or transmits) a waiting screen to the terminal 100 according to the entry of the terminal 100 into the chat room. Here, the waiting screen includes the type of the corresponding chat room, the topic, the chat room name, the keyword, the nickname (or chat name) of the user who enters the corresponding chat room, the chat temperature, and the number of chats (or the number of times the user has participated in the chat room), etc. In this case, the chat temperature may be calculated based on the number of chats, the received evaluation results, etc., and the concept of level or points may be applied instead of the chat temperature. That is, for the level or point, the level or the preset number of points may be calculated according to a plurality of preset grades based on the number of chats, the received evaluation results, etc.

In addition, when the room leader who opens the corresponding chat room selects the preset start menu while the number of people (or the number of participants) set in the chat room related to the corresponding theme practice is met, the terminal 100 is linked with one or more other terminals 100 (not illustrated) owned by one or more other users in the corresponding chat room related to the corresponding theme practice and the server 200 to perform the chat function related to the specific theme (or topic) of the corresponding chat room. In this case, in the case where it is difficult to give an answer related to the theme while performing the chat function for the theme, when the tip menu displayed on one side of the screen of the terminal 100 is selected to check the tip, the terminal 100 may display information (or a tip) on related themes on one side of the screen. In addition, the terminal 100 is linked with the server 200 and uses the avatar (or character) preset in the terminal 100 to change (or apply/reflect) and display the expression (e.g., including happiness, joy, excitement, passion, pleasure, frustration, outrage, sadness, surprise, pain, rage, etc.) of the corresponding avatar according to the analysis (e.g., including a change in a user's gaze, a change in facial expression, a voice tremor, a change in heart rate, etc.) of the psychological state of the corresponding user's voice and/or facial expression while the user of the terminal 100 is chatting.

In addition, when the theme practice (or theme practice chat function) related to the question-and-answer chat room ends, the server 200 is linked with the terminal 100 to perform the self-evaluation function for the user of the terminal 100. Here, the self-evaluation function includes an evaluation function for overall satisfaction item (e.g., including disappointed, average, satisfied, other, etc.) with the user's chat, an anxiety item, a confidence/assertiveness item, a mind control item, a communication skills/logic/persuasive power item, an immersion item, an empathy/listening skills item, etc. In this case, the anxiety item, the confidence/assertiveness item, the communication skills/logic/persuasive power item, the immersion item, the empathy/listening skills item, etc., may be selected in a preset range (e.g., 0 to 100 points) depending on the user's selection.

That is, when the theme practice (or theme practice chat function) related to the question-and-answer chat room ends, the server 200 receives (or collects) the information related to the self-evaluation, the identification information of the terminal 100, etc., from the terminal 100 that participates in the question-and-answer chat room related to the corresponding theme practice and performs the chat function.

In addition, after the user of the terminal 100 performs the chat function through the theme practice with other users, when the theme practice (or theme practice chat function) related to the free theme chat room or the discussion theme chat room ends, the server 200 is linked with the terminal 100 to perform the self-evaluation function for the user of the terminal 100 and the counterpart evaluation function for one or more other users participating in the corresponding chat room. Here, the self-evaluation function includes an evaluation function for overall satisfaction item (e.g., including disappointed, average, satisfied, others, etc.) with the user's chat, an anxiety item, a confidence/assertiveness item, a mind control item, a communication skills/logic/persuasive power item, an immersion item, an empathy/listening skills item, etc. In this case, the anxiety item, the confidence/assertiveness item, the mind control item, the communication skills/logic/persuasive power item, the immersion item, the empathy/listening skills item, etc., may be selected in a preset range (e.g., 0 to 100 points) depending on the user's selection. In addition, the counterpart evaluation function performs a feedback (or feedback evaluation/feedback information) function on the counterpart that participates in the corresponding chat, or includes evaluation functions for chat evaluation items for each user (e.g., including “He doesn't seem anxious at all,” “He's confident and assertive, “He's good at controlling his emotions,” “He's good at communicating logically,” “He's good at immersing himself in the situation,” “He listens well and has good empathy,” etc.), etc. In this case, the terminal 100 may be configured to allow the user to directly input content into the counterpart evaluation function.

That is, when the theme practice (or theme practice chat function) related to the free theme chat room or the discussion theme chat room ends, the server 200 receives (or collects) the information related to the self-evaluation from the plurality of terminals 100 participating in the chat room related to the corresponding theme practice, the information related to the counterparty evaluations by other users, the identification information of the terminal 100, etc.

In addition, the server 200 generates the chat result for the user of the terminal 100 participating in the corresponding chat room according to the performance of the self-evaluation function in the terminal 100 participating in the theme practice related to the question-and-answer chat room. Here, the chat result (or question-and-answer chat room information) includes total average score items according to self-evaluation, an overall rating display item, an item for displaying scores (e.g., including an anxiety score, a confidence/assertiveness score, a mind control score, a communication skills/logic/persuasive power score, an immersion score, an empathy/listening skills score, etc.) for each self-evaluation item and an average score related to the corresponding theme practice, the number of saves display item for displaying the number of times chat content from the corresponding chat room can be saved (or downloaded), a chat recording storing item for storing the corresponding chat content, a chat time display item for displaying a total chat time in the corresponding chat room and the time the user spoke, a chat content output item for re-listening the chat content from the corresponding chat room, etc.

In addition, the server 200 transmits the generated chat results to the first terminal 100.

In addition, the server 200 updates (or calculates/computes) the free expression index, the anxiety index, the chat temperature (or level/point), etc., related to the user based on other chat results generated in relation to the user of the terminal 100.

In addition, when the user of the terminal 100 performs the chat function through the theme practice with other users, the server 200 generates another chat result for each user participating in the corresponding chat room depending on the performance of the self-evaluation function and the counterpart evaluation function performed by one or more terminals participating in the theme practice related to the corresponding free theme chat room or the corresponding discussion theme chat room. Here, the other chat result (or other chat result information) includes total average score items according to self-evaluation, an overall rating display item, an item for displaying scores (e.g., including an anxiety score, a confidence/assertiveness score, a mind control score, a communication skills/logic/persuasive power score, an immersion score, an empathy/listening skills score, etc.) for each self-evaluation item and an average score related to the theme practice display item, an evaluation item received according to the counterpart's evaluation, the number of saves display item for displaying the number of times chat content from the corresponding chat room can be saved (or downloaded), a chat recording storing item for storing the chat content, a chat time display item for displaying the total chat time in the corresponding chat room and the time the corresponding user spoke, a chat content output item for re-listening to the chat content in the corresponding chat room, etc. In addition, the received evaluation item includes the number of votes item for each chat satisfaction item, the number of votes item for each chat evaluation item, an evaluation content item input directly by the counterpart, etc.

In addition, the server 200 transmits the generated other chat result for each user to the terminal 100 owned by each user.

In addition, the server 200 updates (or calculates/computes) the free expression index, the anxiety index, the chat temperature (or level/point), etc., related to the user based on other chat results generated in relation to the user of the terminal 100.

In this way, when the chat functions such as topic talk, role-playing, and theme practice are performed on the terminal 100, the server 200 uses the results (e.g., including self-evaluation information, received evaluation information, etc.) of performing the corresponding chat function to update the “free expression” index, the anxiety index, the chat temperature (or level/points), etc., related to the user.

In addition, the server 200 may not only apply the results (e.g., including self-evaluation information, received evaluation information, etc.) of performing the corresponding chat function, but also apply (or reflect) the detailed emotional record information, the simple emotional record information, etc., recorded by the corresponding terminal 100 along with the results of executing the corresponding chat function to update the “free expression” index, the anxiety index, the chat temperature (or level/points), etc., related to the user.

In addition, when the terminal 100 is invited to the chat room created by the expert terminal 300, the server 200 is linked with the terminal 100 and the expert terminal 300 to allow the terminal 100 and the expert terminal 300 to enter (or participate in) the same chat room.

In addition, the server 200 is linked with the terminal 100 and the expert terminal 300 to provide (or transmit/forward) the questionnaire transmitted from the expert terminal 300 to the terminal 100. Here, the questionnaire includes one or more multiple-choice/subject questions related to a specific situation (or specific topic).

In addition, the server 200 provides (or transmits/forwards) questionnaire response information transmitted from the terminal 100 to the expert terminal 300.

In addition, the server 200 provides (or transmits/forwards) the response result to the questionnaire related to the corresponding questionnaire response information transmitted (or provided) from the expert terminal 300 to the terminal 100 in response to the questionnaire response information. Here, the response result includes a user's response information for each question, correct answer information for each question, the user's earned score compared to the total points, etc.

In addition, the server 200 is linked with the terminal 100 and the expert terminal 300 to perform (or monitor) the psychological counseling function, the speech lesson, the mentor function, etc., with the expert through the chat room generated from the corresponding expert terminal 300.

In addition, after the chat function ends, the server 200 provides (or transmits/forwards) the feedback information transmitted from the expert terminal 300 to the terminal 100. Here, the feedback information includes information such as an expert's view (or opinion) regarding the psychological counseling function, the speech lesson function, the mentor function, etc., performed in the chat room.

The expert terminal 300 communicates with the terminal 100, the server 200, etc. In this case, the expert terminal 300 may be a terminal owned by an expert (or counselor) who wishes to receive a counseling function for a user, a people diary/“my report” confirm function for a specific user while performing the counseling function, a counseling result generation and management function, a speech lesson function, a mentor function (or mentoring function), a psychological counseling function and an XR chat room (or metaverse chat room) creation and management function, etc.

In addition, the expert terminal 300 is linked with the server 200 to subscribe as the expert (or counselor) to receive the counseling function for the user through the dedicated app and/or website provided by the server 200, the people diary/“my report” confirmation function for a specific user while performing the counseling function, the counseling result writing and management function, the speech lesson function, the mentor function (or mentoring function), the XR chat room (or metaverse chat room) writing and management function, etc., and register the personal information, etc., to the server 200. In this case, the personal information includes an ID, an email address, a password (or passcode), a name, a gender, a date of birth, contact information, an address (or address information), an authorized certificate proving expertise etc.

In addition, the expert terminal 300 may subscribe a user as a member on the server 200 using SNS account information to which the user of the expert terminal 300 subscribes or other site account information. Here, the SNS account may be information related to Facebook, Twitter, Instagram, Kakao Story, Naver Blog, etc. In addition, other site accounts may be information related to YouTube, Kakao, Naver, etc. In this case, the dedicated app may be configured as different user interfaces according to a user who receives a chat room search function, a chat function/user-to-user chat function, a counseling with expert function/expert search function, a room creation function, a topic talk chat room function, a role-playing chat room function, a theme practice chat room function, a people diary writing and/or management function, a “my report” management function, etc., and an expert (or counselor) who receives a counseling function for a user, a people diary/my report check function for a specific user while performing the counseling function, a counseling result writing and management function, a speech lesson function, a mentor function (or mentoring function), a psychological counseling function, an XR chat room (or metaverse chat room) creation and management function, etc.

In addition, when performing the member subscription process, the expert terminal 300 should complete an authentication function through an identity authentication method (e.g., including a mobile phone, a credit card, an i-PIN, etc.) to complete the member subscription process for the server 200 normally.

In addition, when subscribing as the expert, the expert terminal 300 may transmit a certificate (or a certificate in the form of a file such as PDF format) that can prove that the expert is the corresponding expert to the server 200, and when the expert verification procedure is completed normally in the corresponding server 200, complete the member subscription process for the server 200 normally.

In this case, the expert terminal 300 is linked with the server 200 to receive the results according to approval, rejection, request for additional supplementation, etc., through the review of the corresponding certificate, and displays (or outputs) the received results.

In addition, after the member subscription is completed, to use the service provided by the server 200, the expert terminal 300 installs a dedicated app (or application/application/specific app) provided from the server 200 on the corresponding expert terminal 300. In this case, the dedicated app includes the native app, the mobile web app, the RWD, the AWD, the hybrid app, etc., and may be an app for performing a counseling function for a user, a people diary/“my report” confirmation function for a specific user while performing the counseling function, a counseling result generation and management function, a speech lesson function, a mentor function (or mentoring function), and an XR chat room (or metaverse chat room) creation and management function, etc.

In addition, after the member subscription is completed, the expert terminal 300 may display a discount coupon provided by the server 200 through the corresponding dedicated app. In this case, the discount coupon may be a discount coupon including discount information of a certain percentage when performing the counseling function, etc., provided by the server 200.

In addition, the expert terminal 300 executes a dedicated app pre-installed on the corresponding expert terminal 300 and displays an app execution result screen according to the execution of the dedicated app. In this case, the app execution result screen includes an expert information display menu (or button/item/area) for displaying information related to the expert of the corresponding expert terminal 300, a dashboard menu for displaying general information related to an expert, a calendar menu for displaying a counseling schedule of the corresponding expert, a member list menu for displaying member information of a user receiving counseling from the corresponding expert, a member report menu for displaying activity details for a preset period of a user receiving counseling from the corresponding expert, a message menu for a message (or post message) function with a user who is receiving counseling from or has a counseling history with the corresponding expert, a “my performance” menu for providing information such as the counseling performance performed by the corresponding expert, a data management menu for managing information on a questionnaire or data related to a user that an expert is counseling or has a counseling history with, a setting menu (or my page menu) for setting user information, a website function, etc., an XR room creation menu for performing a counseling function with a user in an XR or metaverse environment, a detailed information display area (or detailed information display menu) for displaying detailed information (or additional information) according to the menu selection, etc. Here, the expert terminal 300 may perform a login procedure when accessing the website using the ID and password according to member subscription, a barcode or QR code including the ID, etc., while subscribed as a member in the server 200 that provides the dedicated app (or website) to perform one or more functions of the corresponding website (e.g., including a counseling function for a user, a function of confirming people diary/“my report” for a specific user while performing the counseling function, a function of generating and managing counseling results, a speech lesson function, a mentor function (or mentoring function), a psychological counseling function, an XR chat room (or metaverse chat room) creation and management function, etc.). In this case, the dedicated app (or the website) may be configured as different user interfaces according to an expert (or counselor) who receives the counseling function for a user, the function of confirming a people diary/“my report” for a specific user while performing the counseling function, the function of generating and managing counseling results, the speech lesson function, the mentor function (or mentoring function), the XR chat room (or metaverse chat room) creation and management function, etc., and a user (or counseling requester/client) who receives the chat room search function, the chat function/user-to-user chat function, the counseling with expert function/expert search function, the room creation function, the topic talk chat room function, the role-playing chat room function, the theme practice chat room function, the people diary creation and/or management function, the “my report” management function, etc.

In addition, when the preset dashboard menu is selected from the app execution result screen displayed on the expert terminal 300, to display the information related to the expert of the corresponding expert terminal 300, the expert terminal 300 is linked with the server 200 to display a dashboard screen corresponding to the selected dashboard menu. Here, the dashboard screen includes an immediate counseling menu for performing a counseling function with a user (or client) in real time while accessing a website on the expert terminal 300, a search menu for searching for functions or menus corresponding to the search word, a notification menu for confirming notices or notification messages, etc., a user information display menu for displaying user information (e.g., including an expert character/picture, an expert name/name, etc.), a calendar item (or calendar menu/area/screen) for displaying an expert's counseling schedule by day, week, month, etc., a “my profile card” menu for displaying an expert's profile card, a counseling request approval count menu for displaying the number of counseling request approvals in relation to counseling requests requested from the corresponding expert, the total number of reserved counseling sessions display menu for displaying the total number of counseling sessions requested from the corresponding expert, a number of new members this month display menu for displaying the number of users who requested new counseling sessions from the expert in that month, a total number of members display menu for displaying the total number of users who have requested counseling from the corresponding expert, a member list item (or member list menu/area/screen) for displaying member information of the user receiving counseling from the corresponding expert, etc. In this case, when accessing the dedicated website on the expert terminal 300 (when logged into the website), the expert terminal 300 may display a dashboard screen corresponding to the dashboard menu set by default in the detailed information display area.

In addition, when the preset calendar menu is selected from the app execution result screen displayed on the expert terminal 300, the expert terminal 300 is linked with the server 200 to display a calendar screen corresponding to the selected calendar menu to display the counseling schedule of the corresponding expert. Here, the calendar screen includes a calendar type menu for selecting the type of calendar provided (e.g., including list, monthly, weekly, etc.), an immediate counseling menu for performing a counseling function with the user (or client) in real time while accessing the website on the expert terminal 300, a search menu for searching for functions or menus corresponding to the search word, a notification menu for confirming notices or notification messages, etc., a user information display menu for displaying user information (e.g., including an expert character/picture, an expert name/name, etc.), a detailed counseling schedule menu for displaying the counseling schedule of the expert according to the calendar type and selected period selected in the calendar type item, a counseling request approval menu for providing information on one or more counseling requests for which the expert has not yet processed counseling request approval among the counseling requests requested from the corresponding expert, an available reservation time menu for setting the available counseling reservation date and time zone of the corresponding expert, an output menu for outputting the expert's counseling schedule displayed on the calendar screen, a filter menu for providing a filter function for the expert's counseling schedule, a counseling addition menu for adding a new counseling schedule, etc. In this case, the calendar type item may be in a state in which a weekly item is selected by default. In addition, the detailed counseling schedule item may display (or include) some of the counseling schedules reserved for a specific date and time zone (e.g., including a user/client's nickname, a reservation time zone, etc.) when the counseling schedule is reserved for the specific date and time zone, and separately display a date and time zone that are available for reservation and a date and time zone that are not available for reservation among all dates and time zones depending on the expert setting (e.g., including different background colors and/or background patterns, etc., for each date and time zone that are available for reservation and are not available for reservation). In addition, examples of the output item include a print item through a printer (not illustrated), a print (or store/output) item in a file format such as a PDF format or an HWP format, etc.

In addition, when the preset member list menu is selected from the app execution result screen displayed on the expert terminal 300, the expert terminal 300 is linked with the server 200 to display a member list screen corresponding to the selected member list menu to display the member information of the user receiving counseling from the corresponding expert. Here, the member list screen includes a member list type menu for selecting the member list type provided (e.g., including list, thumbnail, etc.), an immediate counseling menu for performing a counseling function with a user (or client) in real time while accessing the website on the expert terminal 300, a search menu for searching for functions or menus corresponding to the search word, a notification menu for confirming notices or notification messages, etc., a user information display menu for displaying user information (e.g., including expert character/picture, expert name/name, etc.), a number of current counseling members display menu for displaying the number of members receiving counseling from the corresponding expert, a sorting menu for providing a sorting function for the member list (e.g., including the order of the earliest next reservation date, the most recently completed counseling date, the order of the longest membership period remaining, the order of the shortest membership period remaining, the order of the first registration date, etc.), a member search menu for searching for members, a counseling request approval menu for providing information on one or more counseling requests for which the expert has not yet processed counseling request approval among the counseling requests requested from the corresponding expert, a filter menu for providing a filter function for the expert's counseling schedule, an output menu for outputting a member list displayed on the member list screen, a deletion menu for deleting information on a specific member displayed on the member list screen, a detailed member list display area (or detailed member list display menu) for displaying information on a user (or client) who has registered with (or is receiving counseling from) the expert according to the member list type selected in the member list type item, etc. In this case, the member list type menu may be in a state in which a list item is selected as default.

In addition, when the preset member report menu is selected from the app execution result screen displayed on the expert terminal 300, the expert terminal 300 is linked with the server 200 to display a member report screen corresponding to the selected member list menu to display the activity details for a preset period of the user receiving counseling from the corresponding expert. Here, the member report screen includes an immediate counseling menu for performing a counseling function with a user (or client) in real time while accessing the website on the expert terminal 300, a search menu for searching for functions or menus corresponding to the search word, a notification menu for confirming notices or notification messages, etc., a user information display menu for displaying user information (e.g., including an expert character/picture, an expert name/name, etc.), a sorting menu for providing a sorting function (e.g. currently online, most recent activity, high “free expression” index, low “free expression” index, etc.) for the member report in order to provide a member report based on activity details for a preset period (e.g. 7 days), a member search menu for searching for members, a counseling request approval menu for providing information on one or more counseling requests for which the expert has not yet processed counseling request approval among the counseling requests requested from the corresponding expert, a filter menu for providing a filter function for the expert's counseling schedule, an output menu for outputting information displayed on the member report screen, a deletion menu for deleting information on a specific member displayed on the member report screen, a detailed member report display area for displaying information on one or more members that meet the sort item, etc. In addition, the detailed member report display area (or detailed member report detail menu) is configured in the form of a profile card, and includes a fixed setting item for selecting whether to fix the profile card of the corresponding counseling requester, a character (or photo/image) of a counseling requester, a nickname item (or name item), a total counseling time item (or total played time item), a “free expression” index item, etc. In addition, the “free expression” index item includes a total “free expression” index (or average “free expression” index) item, a topic talk “free expression” index item, a role-playing “free expression” index item, a theme practice “free expression” index item, etc. In addition, the “free expression” index represents an evaluation index for the user (or counseling requester) of the terminal 100 according to the performance of the chat function, and may be calculated by adding up the average score of the user's self-evaluation after performing the chat function in the chat room (e.g., including the topic talk chat room, the role-playing chat room, the theme practice chat room, etc.) in which the user participates, in relation to one or more chat rooms in which the user of the corresponding terminal 100 participates for a preset period or all chat rooms in which the user of the corresponding terminal 100 participates, and results (or evaluation received) (e.g., +1 for affirmative evaluation/satisfied, −1 for negative evaluation/not great) of evaluation of the user by the counterpart in the chat room in which the user participates.

In addition, when a preset message menu is selected from the app execution result screen displayed on the expert terminal 300, to provide a message (or post message) function with a user who is receiving counseling from the corresponding expert or has a counseling history with the corresponding expert, the expert terminal 300 is linked with the server 200 to display a message screen corresponding to the selected message menu. Here, the message screen includes an immediate counseling menu for performing a counseling function with a user (or client) in real time while accessing the website on the expert terminal 300, a search menu for searching for functions or menus corresponding to the search word, a notification menu for confirming notices or notification messages, etc., a user information display menu for displaying user information (e.g. including an expert character/picture, an expert name/name, etc.), a member search menu for searching for members, a message list menu for providing a list of one or more counseling requesters who have exchanged messages with the expert, when a specific counseling requester (or the nickname of the counseling requester) is selected from the list of counseling requestors included in the message list, a message content display menu for displaying the content of the message exchanged with the specific counseling requester, a detailed personal information display menu for displaying personal information on the specific counseling requester, etc. In addition, the detailed personal information display menu includes a current online state display item for displaying information on the real-time connection state of the person requesting counseling, a character (or image/photo) item of a person requesting counseling, a nickname, a gender, a date of birth, an emergency contact network (or phone number) of the person requesting counseling, a counseling ticket type item (e.g., time, number of sessions, etc. for each type), a first registration date item, an average counseling time display item, the most recent counseling date display item (or the most recently completed counseling date display item), a next reservation date display item (or counseling reservation date display item), a deletion item for deleting content of messages with the person requesting counseling, a report item for reporting content of messages with the person requesting counseling, etc.

In addition, when the preset “my performance” menu is selected from the app execution result screen displayed on the expert terminal 300, to provide information such as the counseling performance performed by the corresponding expert, the expert terminal 300 is linked with the server 200 to display a “my performance” screen corresponding to the selected my performance menu. Here, the “my performance” screen is an immediate counseling menu for performing a counseling function with a user (or client) in real time while accessing the website on the expert terminal 300, a search menu for searching for functions or menus corresponding to the search word, a notification menu for confirming notices or notification messages, etc., a user information display menu for displaying user information (e.g., including an expert character/picture, an expert name/name, etc.), a “my performance” period menu for selecting the type of “my performance” provided (e.g., including, weekly, monthly, yearly, etc.), a total number of counseling ticket purchase case display menu according to “my performance” type selected in the “my performance” period menu, a counseling ticket repurchase rate display menu, a total revenue display menu, a total number of members consulted display menu, a monthly new counseling member information display menu, a recently registered member display menu for displaying information on a preset number of (e.g., 6) recently registered members, a monthly counseling performance display menu, a detail menu for displaying counseling performance details, a menu for displaying an expert's average rating (or rating/star rating), a menu for displaying the total number of reviews, a menu for displaying the number of new reviews for a preset period (e.g., the last 7 days), etc.

In addition, when the preset data management menu is selected from the app execution result screen displayed on the expert terminal 300, to manage the information on questionnaires or materials related to users with whom the expert is consulting or has a counseling history, the expert terminal 300 is linked with the server 200 to display a data management screen corresponding to the selected data management menu. Here, the data management screen includes an immediate counseling menu for performing a counseling function with the user (or client) in real time while accessing the website on the expert terminal 300, a search menu for searching for functions or menus corresponding to the search word, a notification menu for confirming notices or notification messages, etc., a user information display menu for displaying user information (e.g., including an expert character/picture, an expert name/name, etc.), a number of questionnaires held display menu for displaying the number of questionnaires held by the expert in relation to the counseling requester in counseling, an amount of data held display menu for displaying the amount of data held by the expert in relation to the person requesting counseling, a member search menu for searching for members, an additional menu for adding questionnaires or data held by the expert, an output menu for outputting the information displayed on the data management screen, a deletion menu for deleting specific information displayed on the data management screen, a data information display menu related to the counseling requester being managed by the corresponding expert, etc. In addition, the data information display menu includes a file name item, a format item (e.g., including a questionnaire, data, etc.), a writer item (or expert item), a last modified date item, a file size item, etc., according to the data type.

In addition, when the preset setting menu is selected from the app execution result screen displayed on the expert terminal 300, to set the user information or dedicated app function, etc., the expert terminal 300 is linked with the server 200 to display the setting screen corresponding to the selected setting menu.

In this way, the expert terminal 300 displays a screen related to a specific menu selected from the corresponding app execution result screen, and additionally performs a specific function according to the expert input (or expert selection/touch/control) on the displayed screen.

In addition, when the counseling can be performed on the expert terminal 300 in real time, the expert terminal 300 may set the counseling content, composition, time, usage fee, etc. while executing the corresponding dedicated app.

Accordingly, the terminal 100 that requests counseling from the corresponding expert terminal 300 through the real-time counseling function, speech lesson function, or mentor function and the server 200 may be linked to perform the counseling function, the speech lesson function, or the mentor function in real time.

In addition, when the expert terminal 300 wants to register (or set) information on an available counseling time zone by date, the expert terminal 300 may be linked with the server 200 to set the counseling content, the composition, the available counseling time zone, the usage fee, etc.

In addition, when the preset XR room creation menu (or metaverse room creation menu) is selected from the app execution result screen displayed on the expert terminal 300, to perform the counseling function with the expert in the XR, the expert terminal 300 is linked with the server 200 to display the XR room creation screen (or metaverse room creation screen) corresponding to the selected XR room creation menu.

In addition, the expert terminal 300 is linked with the server 200 to create (or open) the XR chat room (or chat room/metaverse chat room) based on a plurality of input values according to the user input corresponding to the plurality of input items included in the corresponding XR room creation screen.

In addition, the expert terminal 300 is linked with the server 200 to invite a random user to the corresponding created (or opened) XR chat room.

In addition, when the terminal 100 related to the user who responded to the invitation enters the created (or opened) XR chat room, the expert terminal 300 transmits (or provides) the questionnaire created (or written/configured) by the expert terminal 300 to the terminal 100 via the server 200. Here, the questionnaire includes one or more multiple-choice/subject questions related to a specific situation (or specific topic).

In addition, the expert terminal 300 receives questionnaire response information transmitted from the terminal 100 in response to the previously transmitted questionnaire. Here, the questionnaire response information includes response information (e.g., not at all, no, average, yes, very much, etc.) for each question.

In addition, the expert terminal 300 generates a response result based on the received questionnaire response information. Here, the response result includes a user's response information for each question, correct answer information for each question, and the user's earned score compared to the total points, etc. In this case, the response result to the questionnaire response information may be generated by the server 200 based on the preset correct answer information for the corresponding questionnaire.

In addition, the expert terminal 300 transmits the generated response result (or a response result to a questionnaire related to the corresponding questionnaire response information) to the terminal 100.

In addition, the expert terminal 300 is linked with the terminal 100 and the server 200 to perform the psychological counseling function, the speech lesson, the mentor function, etc., with the expert through the chat room generated from the corresponding expert terminal 300.

In addition, after the terminal 100 ends the chat function, the expert terminal 300 generates the feedback information according to the performance of the corresponding chat function. Here, the feedback information includes information such as an expert's view (or opinion) regarding the psychological counseling function, the speech lesson function, the mentor function, etc., performed in the chat room.

In addition, the expert terminal 300 provides the generated feedback information to the terminal 100.

In addition, while consulting with the terminal 100 through the XR counseling room, the expert terminal 300 enters, as the spectator or chatter, the metaverse chat room selected according to the corresponding expert selection from among a new metaverse chat room or one or more pre-created metaverse chat rooms with the corresponding terminal 100 to monitor the chat function between the user of the terminal 100 and other users and provide the feedback information according to the monitoring to the terminal 100.

In addition, when the expert terminal 300 performs the counseling function, the speech lesson function, the mentor function, etc., with the terminal (100) that has previously written (or registered) the personal information use consent form in the form of the electronic document, the expert terminal 300 confirms (or analyzes) the exact state of the user of the terminal 100 while confirming the personal information related to the user of the corresponding terminal 100 during the counseling/lesson/mentoring, the counseling record sheet (or counseling record information), the people diary (or people diary information), the chat report (or “my report” information), the psychological test results, etc., in real time, and provides an appropriate response plan accordingly. In this case, the digital rights management (DRM) function may be set so that the personal information related to the user provided to the expert terminal 300, the counseling record sheet (or counseling record information), the people diary (or people diary information), the chat report (or “my report” information), the psychological test results, etc., may not be stored, but can only be viewed.

In addition, when the expert terminal 300 performs the counseling function, the speech lesson function, the mentor function, etc., with the terminal 100, the expert terminal 300 participates in the chat room, in which a chat function is being performed with other users in the corresponding terminal 100, in the spectator mode to observe the user of the terminal 100 and provide the customized advice and analysis information to the user of the terminal 100 based on the observation results.

The embodiment of the present invention describes performing a people diary/“my report” check function for a specific user, a counseling result writing and management function, a speech lesson function, a mentor function (or mentoring function), a psychological counseling function, an XR chat room (or metaverse chat room) creation and management function, etc., while the expert terminal 300 is performing a counseling function for the user in the form of the dedicated app, but is not limited thereto, and may perform the counseling function for the user, the people diary/“my report” check function for the specific user while performing the counseling function, the counseling result writing and management function, the speech lesson function, the mentor function (or mentoring function), the XR chat room (or metaverse chat room) creation and management function, etc., through the website, etc., provided by the server 200 in addition to the dedicated app.

In this way, when the preset theme practice menu is selected from the app execution result screen, in situations where there is a spectator or in situations where there is no spectator focusing on the situations in which the user of the terminal shows interest, it is possible to perform the group counseling or the group training through the metaverse chat room with the free theme or discussion theme as a topic.

Also, in this way, when the preset “my report” menu is selected from the app execution result screen, it is possible to provide the analysis results according to the performance of the functions such as topic talk, role-playing, and theme practice in relation to the user of the terminal and provide the participation list related to the chat list in which the user participates.

In addition, in this way, when the preset topic talk menu is selected from the app execution result screen, it is possible to perform the chat function with other users of terminals focusing on the specific topic in which the user of the terminal is interested and keywords related to the specific topic, and provide the chat results based on the self-evaluation and counterpart evaluation.

In addition, in this way, when the preset role-playing menu is selected from the app execution result screen, it is possible to perform the chat function according to the role-playing with other users of terminals focusing on the specific topic in which the user of the terminal is interested and keywords related to the specific topic, and provide the chat results based on the self-evaluation and counterpart evaluation.

In addition, in this way, when the preset theme practice menu is selected from the app execution result screen, it is possible to provide the feedback related to the situations in which the user of the terminal is interested either alone or in collaboration with other users of terminals and provide the chat results based on the self-evaluation and counterpart evaluation according to the feedback.

In addition, in this way, when the preset people diary menu is selected from the app execution result screen, the user of the terminal can record the emotions felt in a specific situation today in the diary format.

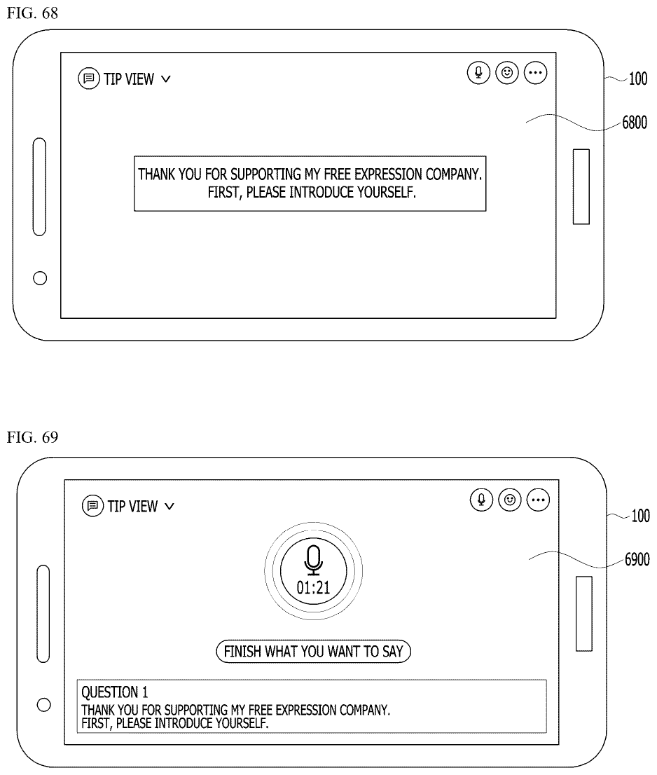

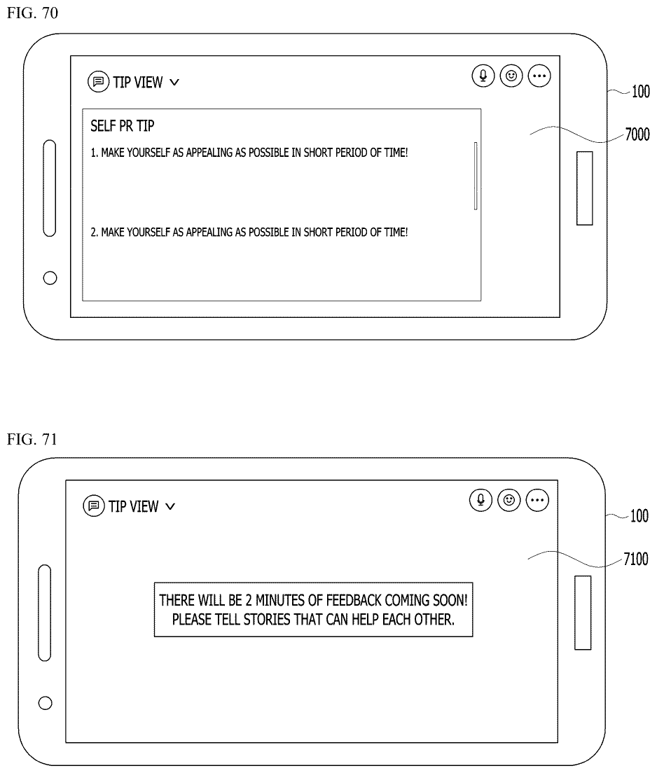

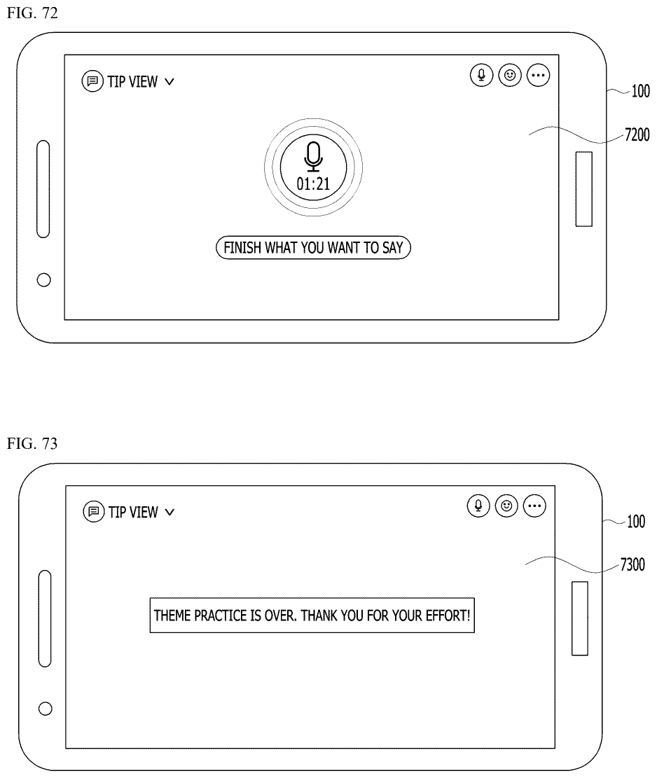

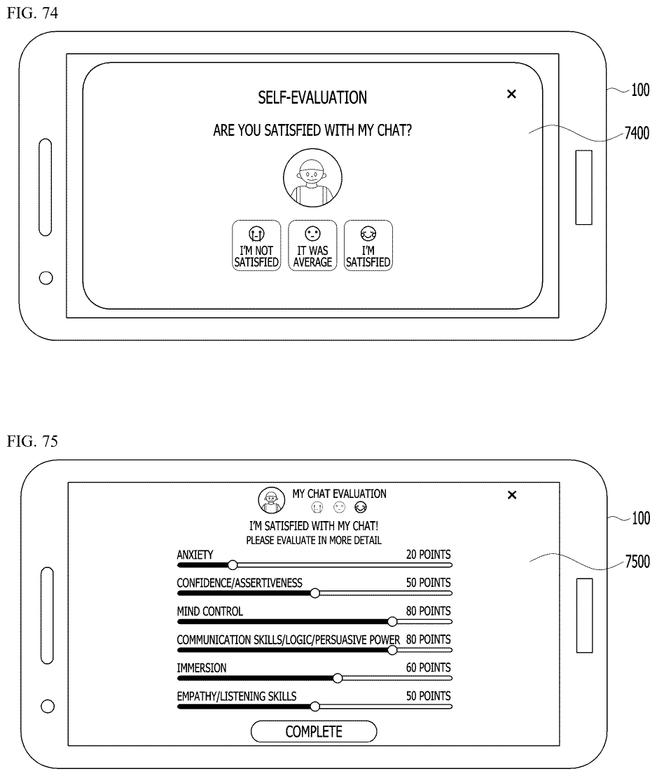

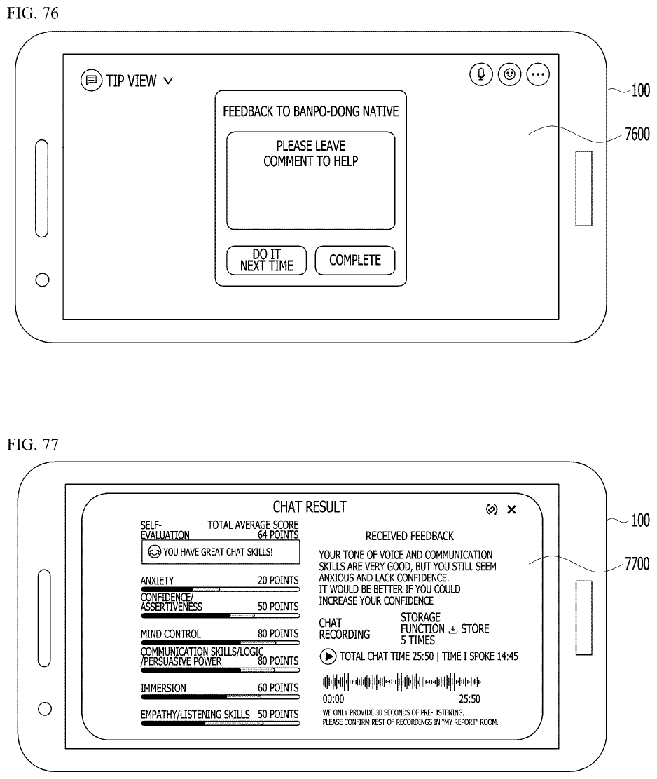

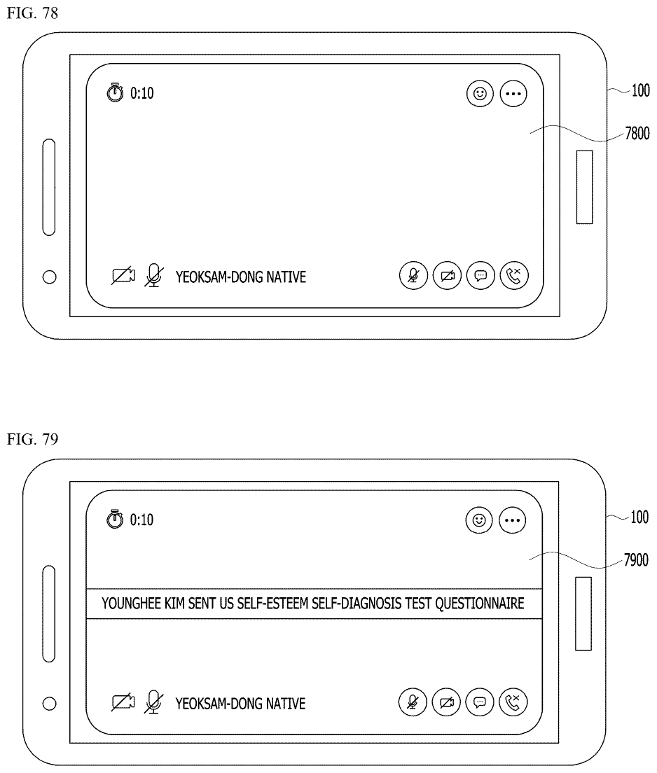

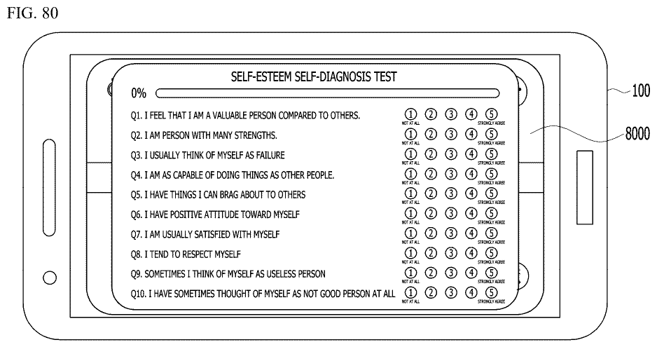

Hereinafter, the method of providing psychological counseling according to the present invention will be described in detail with reference to FIGS. 1 to 80.

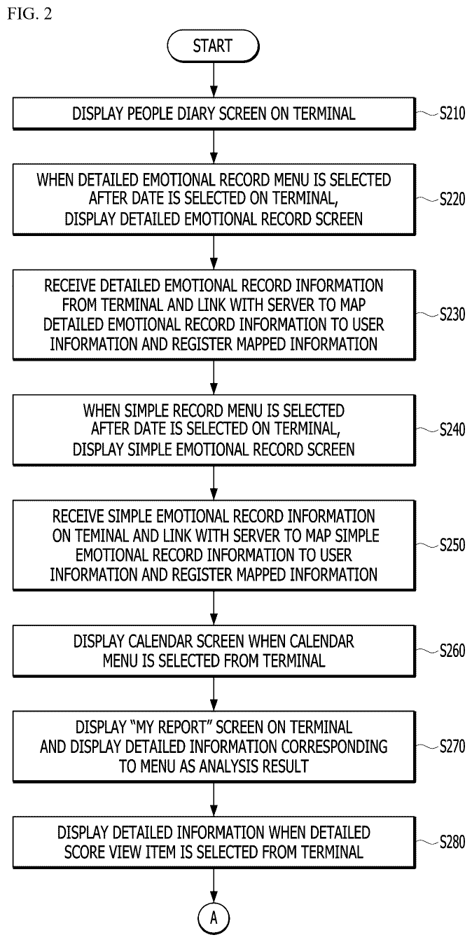

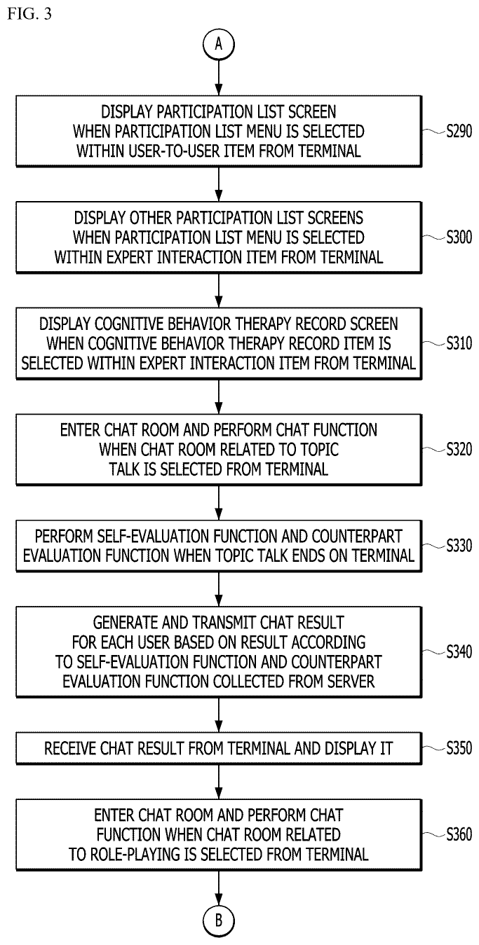

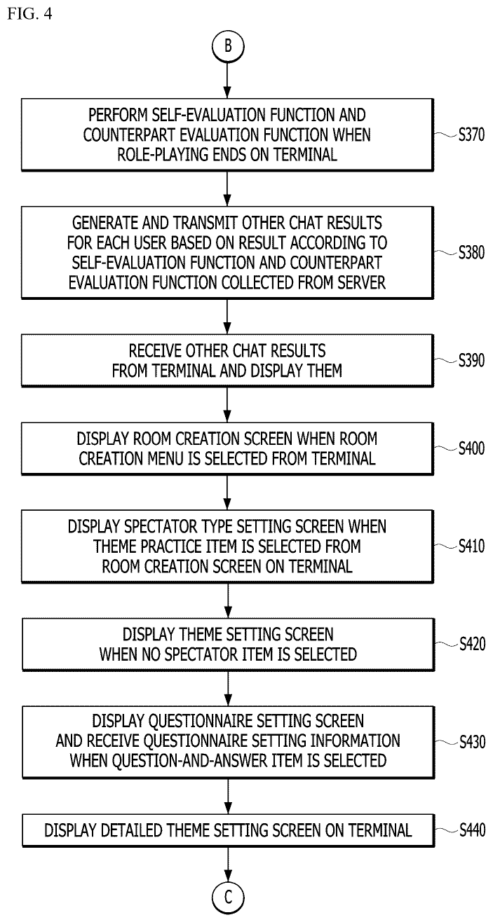

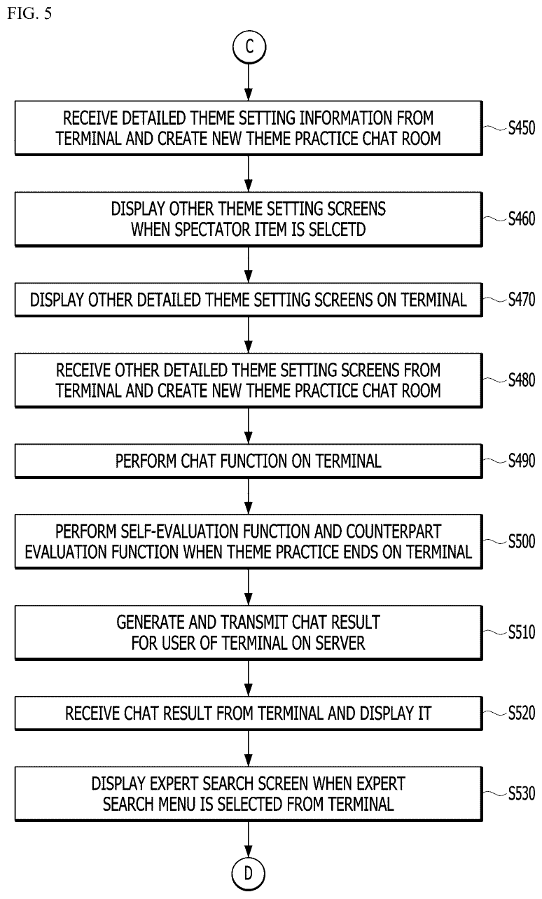

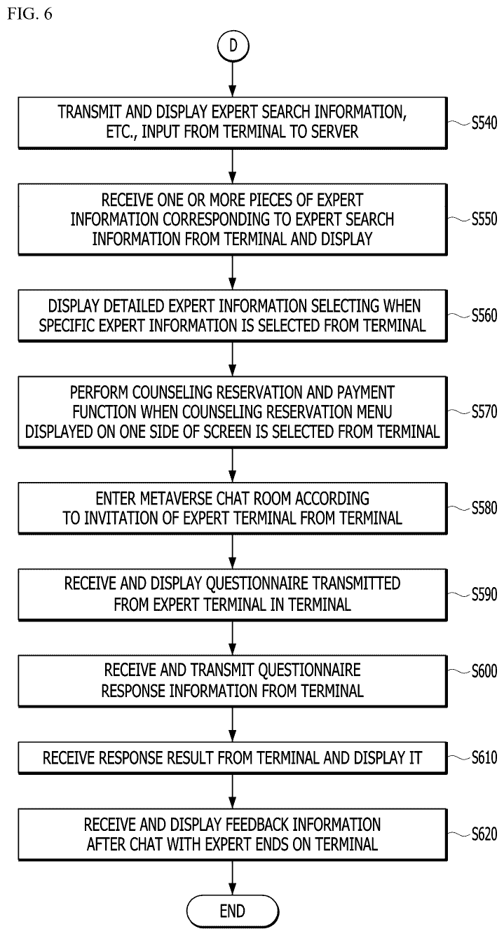

FIGS. 2 to 6 are flowcharts illustrating a method of providing psychological counseling according to an embodiment of the present invention.

First, the terminal 100 executes a dedicated app pre-installed on the terminal 100 and displays an app execution result screen according to the execution of the dedicated app. In this case, the app execution result screen includes an edit menu (or button/item) for screen editing, a search menu (or expert search menu) for searching for the chat room corresponding to the search word or searching for an expert, a notification menu for confirming notices or notification messages, etc., a “my page” menu (or setting menu) for setting a user information or dedicated app function, etc., an advertising display area (or advertising/event display menu) where advertising content or event content is displayed, a subscription menu for regular subscription to chat room service, a user-to-user menu for performing chat functions corresponding to topic talk, role-playing, theme practice, etc. with other users related to a specific topic or situation, an expert interaction menu for performing psychological counseling or speech lesson functions with an expert, a chat room display area (or chat room display menu) for displaying a list of chat rooms opened in relation to a preset category, a filter menu for providing a filter function for the list of opened chat rooms, a view only waiting rooms menu for providing a list of waiting chat rooms, a random participation menu for randomly participating in a chat room among the opened chat rooms, a room creation menu for creating a new chat room of one's choice, a home menu for moving to a home screen/home screen, a people diary menu for writing an emotional state by date related to a user, a “my report” menu for providing analysis results based on performance of a user's chat function, participation list, and cognitive behavior therapy record (or cognitive behavior record), etc. In this case, the user-to-user menu (or user-to-user tab) includes an analysis result item for providing analysis results according to the performance of functions such as topic talk, role-playing, and theme practice in relation to the user of the terminal 100, a participation list item for providing a participation list related to the chat list in which the user participates, etc. In addition, the expert interaction menu (or expert interaction tab) includes a participation list menu for providing a list of people who have performed a psychological counseling function, a speech lesson function, or a mentoring function with an expert in relation to a specific topic or situation, a cognitive behavior therapy record menu for providing expert feedback based on performance of a training function on appropriate chat room methods in relation to the specific topic or situation, etc. In addition, the filter screen includes a topic talk filter item (e.g., interpersonal relationship, daily life, employment/workplace, knowledge sharing, well-being, psychology/MBTI, travel, art, sports, other, etc.) related to topic talk, a role-playing filter item (e.g., including work, dating, daily life, other, etc.) related to role-playing, a theme practice filter item (e.g., including blind dates, phone situations, interview situations, work reports, small-scale presentations, large-scale presentations, professional discussions, pros and cons discussion, other, etc.) related to theme practice, etc. Here, the terminal 100 performs a login procedure when running the dedicated app using the ID and password according to the membership subscription, a barcode or a QR code including the ID, etc., while subscribing as a member in the server 200 that provides the dedicated app to perform one or more functions of the dedicated app (e.g., including a chat room search function, a chat function/user-to-user chat function, a counseling with expert function/expert search function, a room creation function, a people diary writing function, a “my report” checking function, etc.). In this case, the dedicated app may be configured as different user interfaces according to a user who receives a chat room search function, a chat function/user-to-user chat function, a counseling with expert function/expert search function, a room creation function, a topic talk chat room function, a role-playing chat room function, a theme practice chat room function, a people diary writing and/or management function, a “my report” management function, etc., and an expert (or counselor) who receives a counseling function for a user, a people diary/“my report” check function for a specific user while performing the counseling function, a counseling result writing and management function, a speech lesson function, a mentor function (or mentoring function), a psychological counseling function, an XR chat room (or metaverse chat room) creation and management function, etc.

In addition, when the preset people diary menu is selected from the app execution result screen displayed on the terminal 100, the terminal 100 displays the people diary screen corresponding to the selected people diary menu to write the emotional state by date related to the user. Here, the people diary screen includes a date selection menu for selecting a date, when there is a pre-stored memo (or diary/emotional record content) corresponding to the activated (or selected) date, a memo content display area (or memo content display menu) for displaying the corresponding memo content, a detailed emotional record menu for recording detailed emotions, a simple record menu for simply recording emotions, a calendar menu for confirming an emotional record state by date, etc. In this case, the current date (or today's date) in the corresponding terminal 100 may be displayed by default in the date selection item.

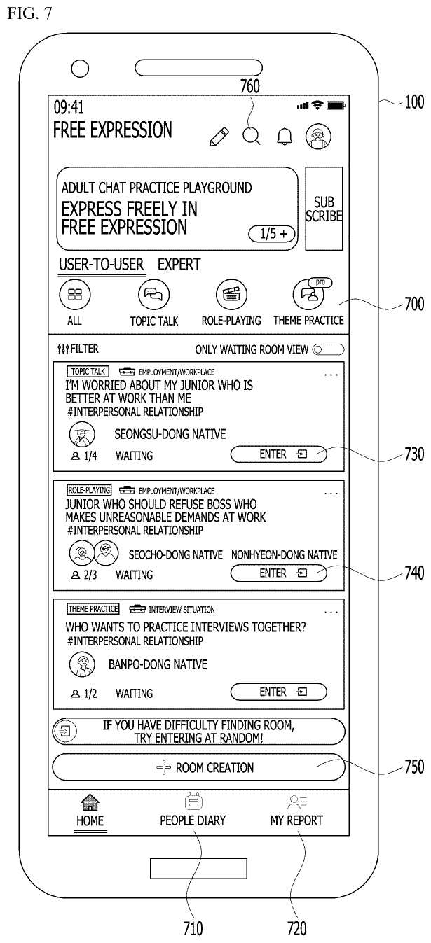

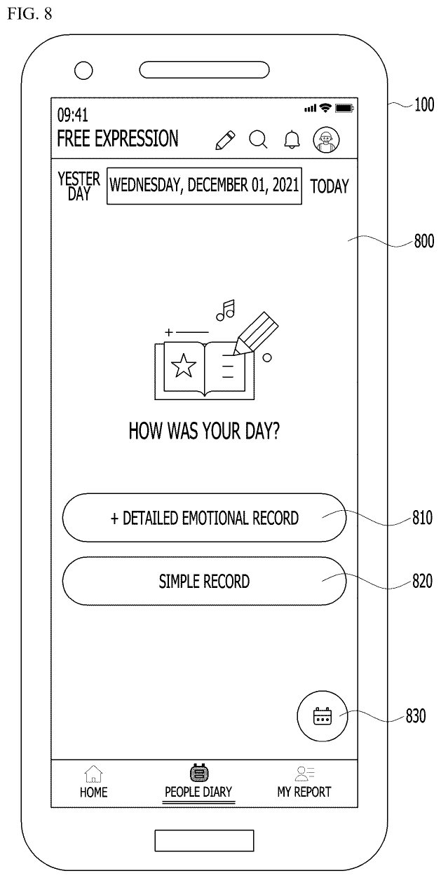

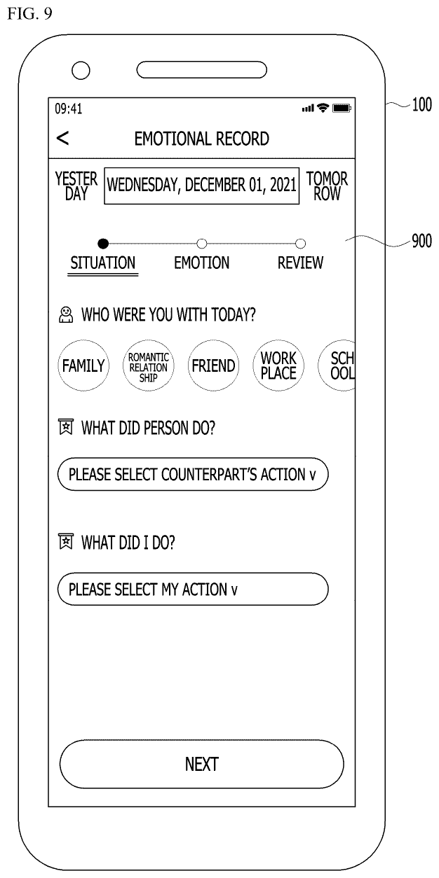

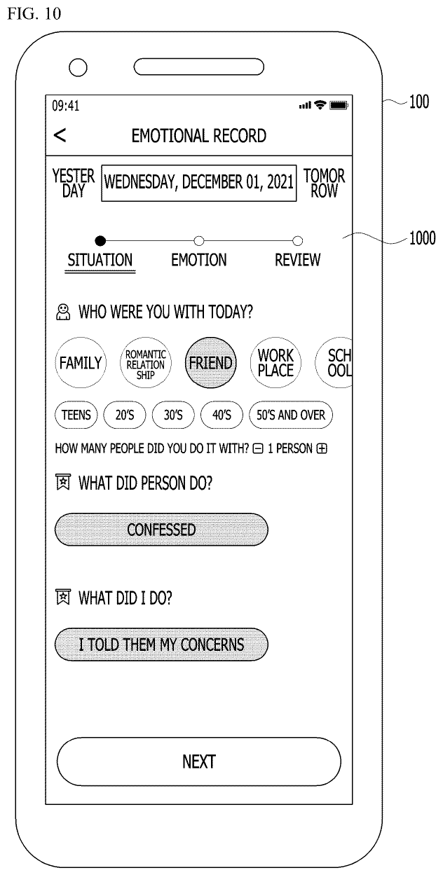

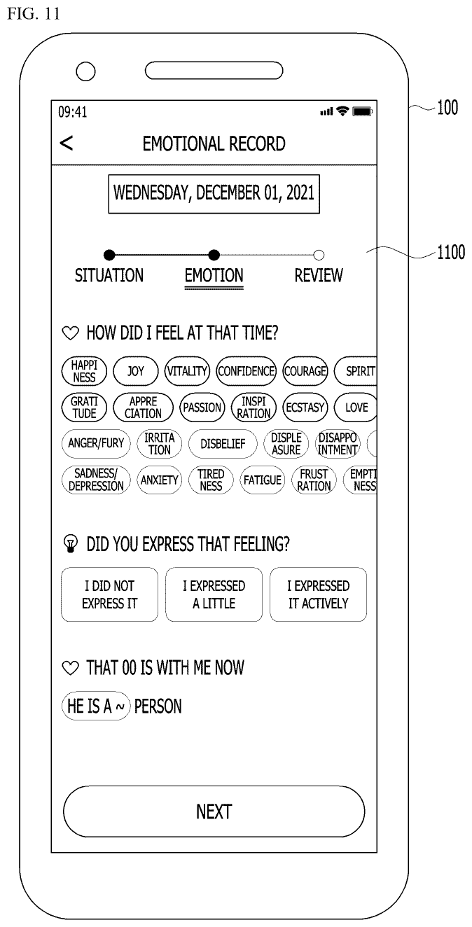

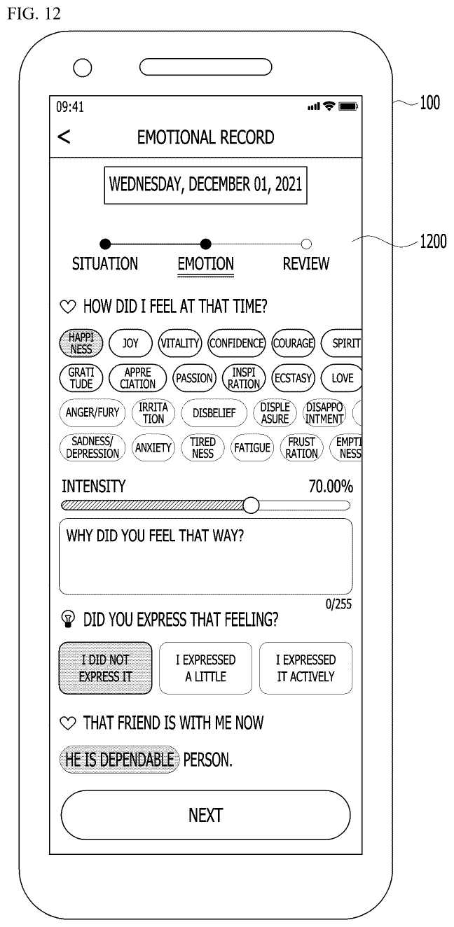

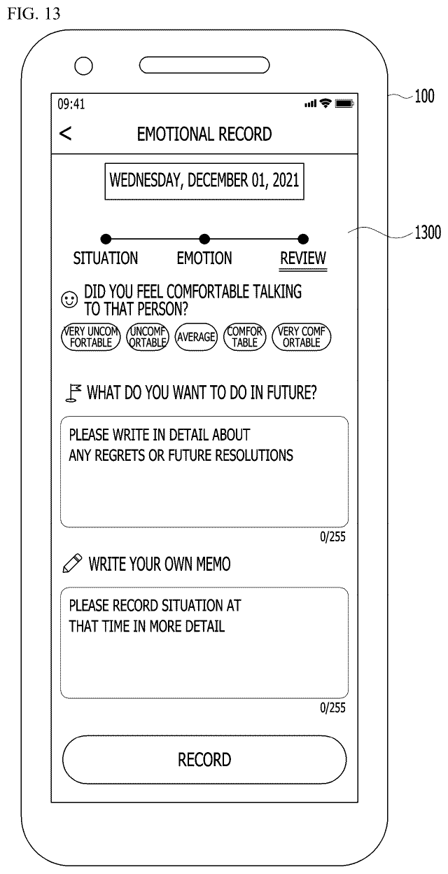

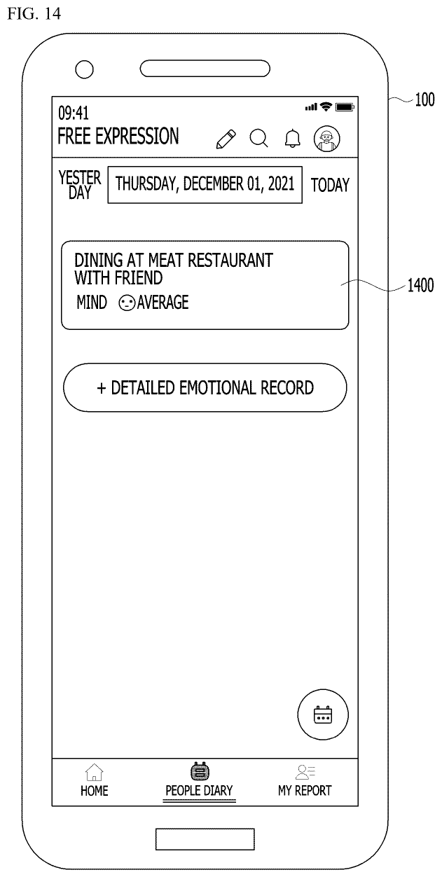

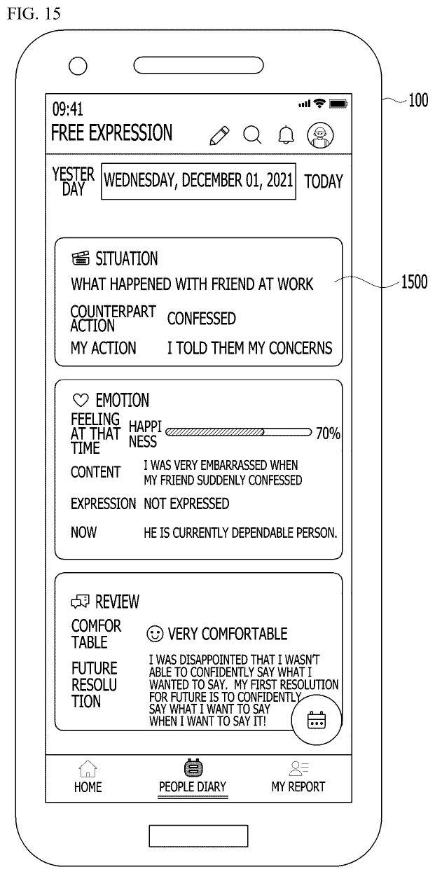

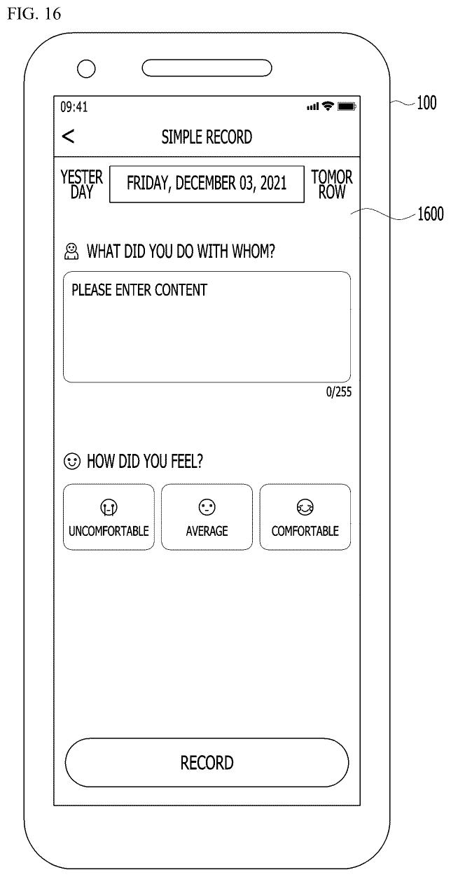

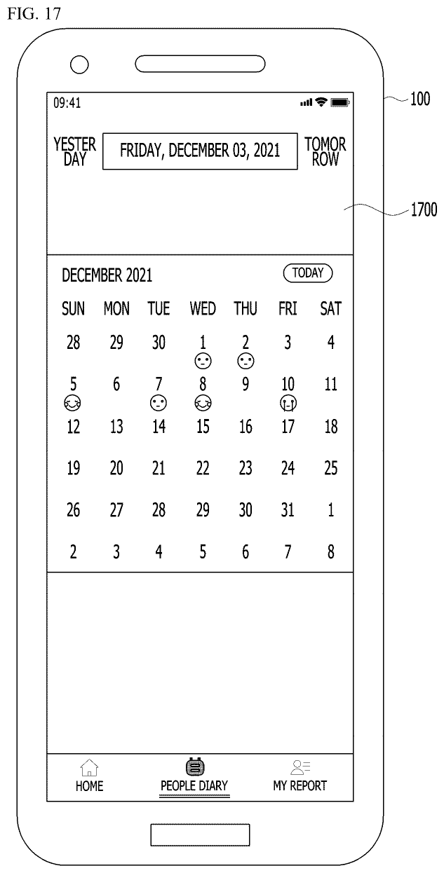

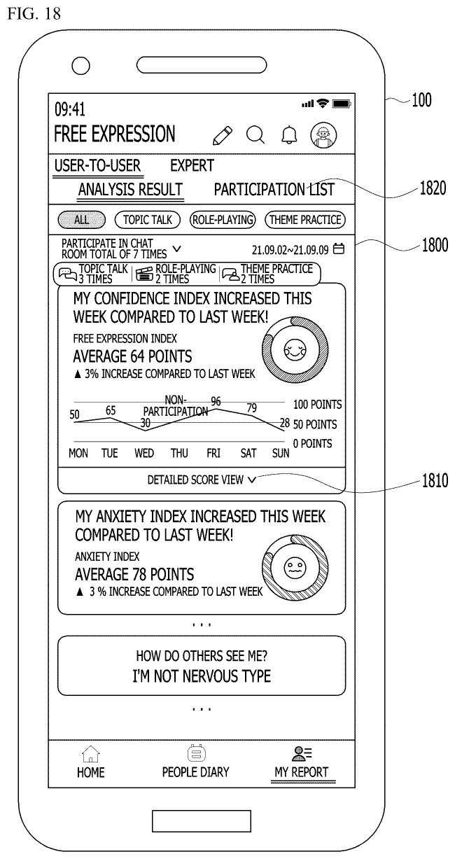

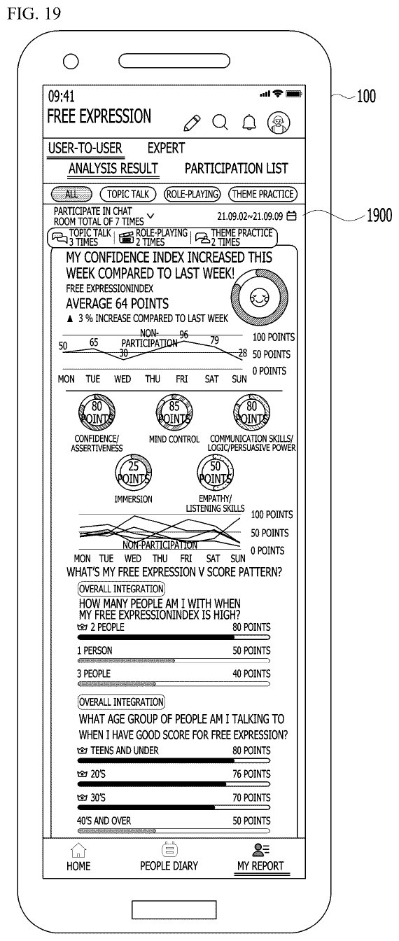

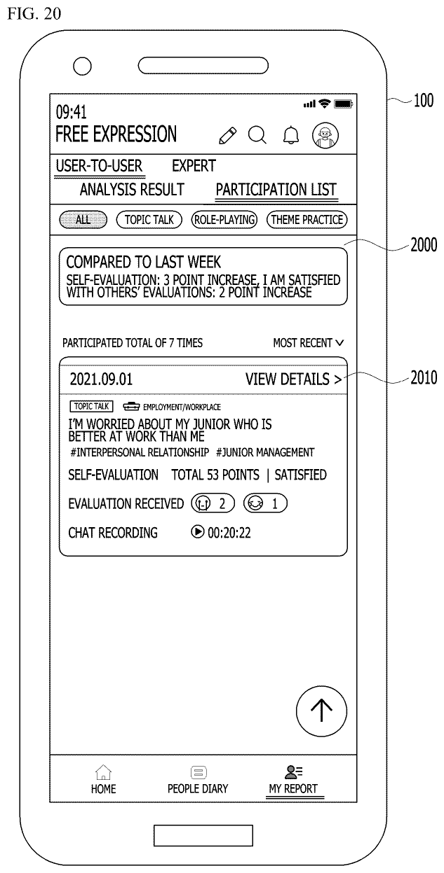

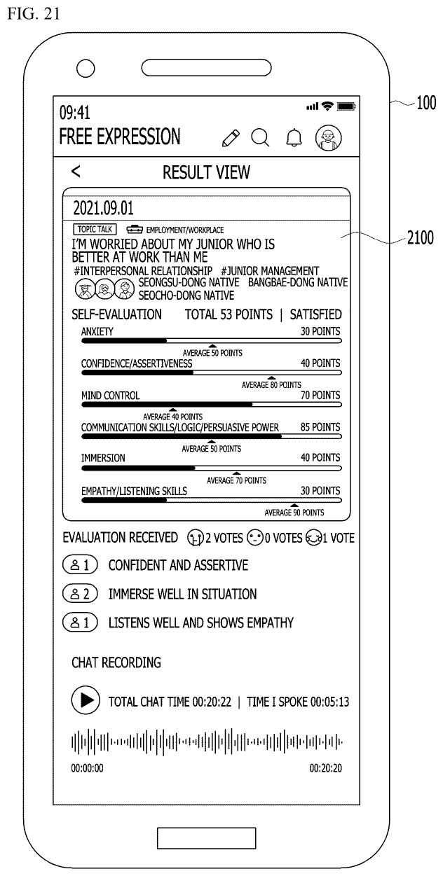

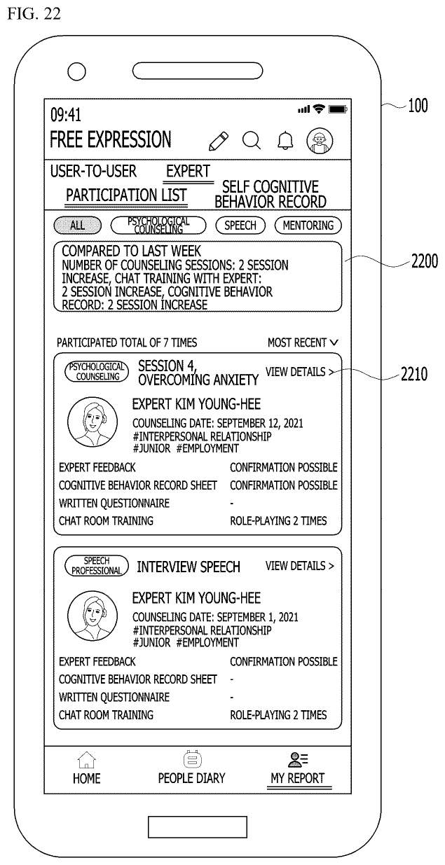

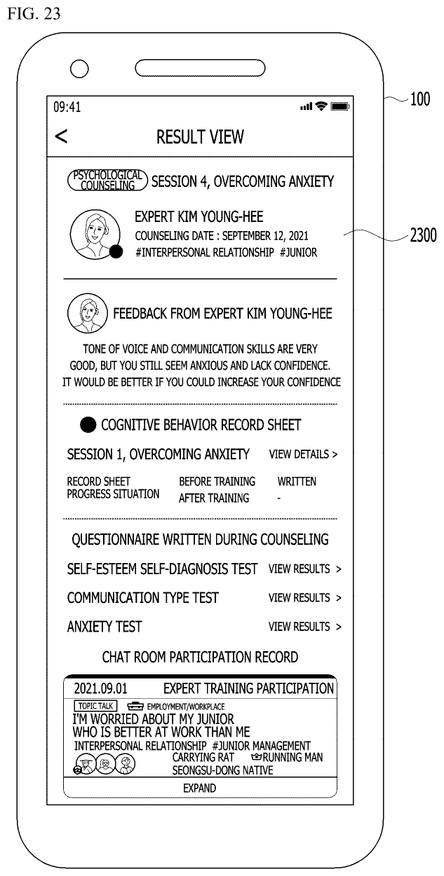

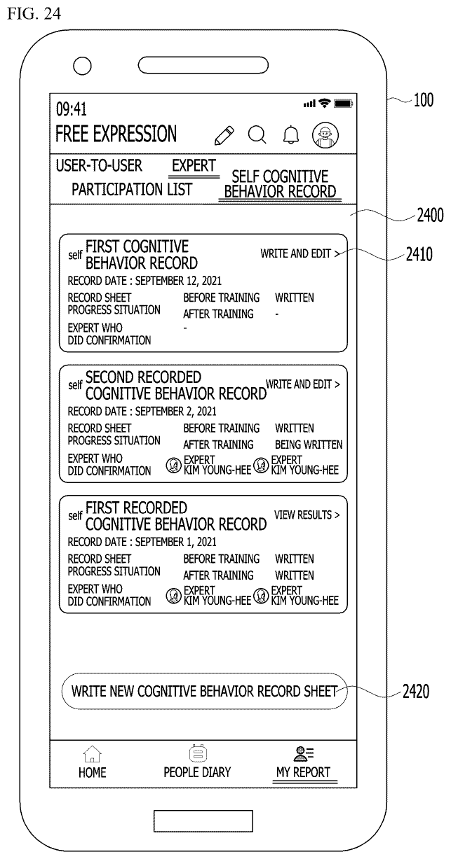

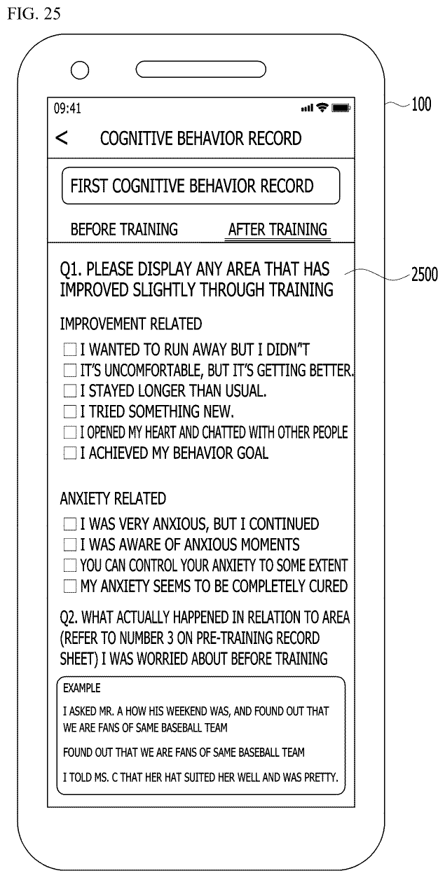

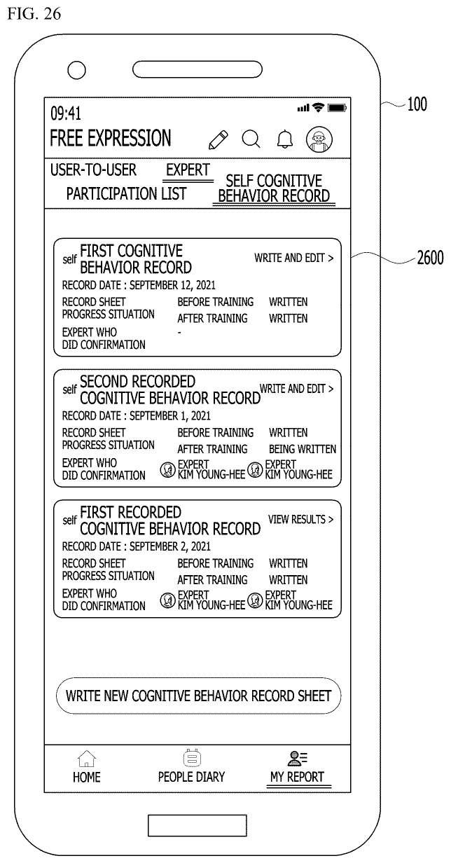

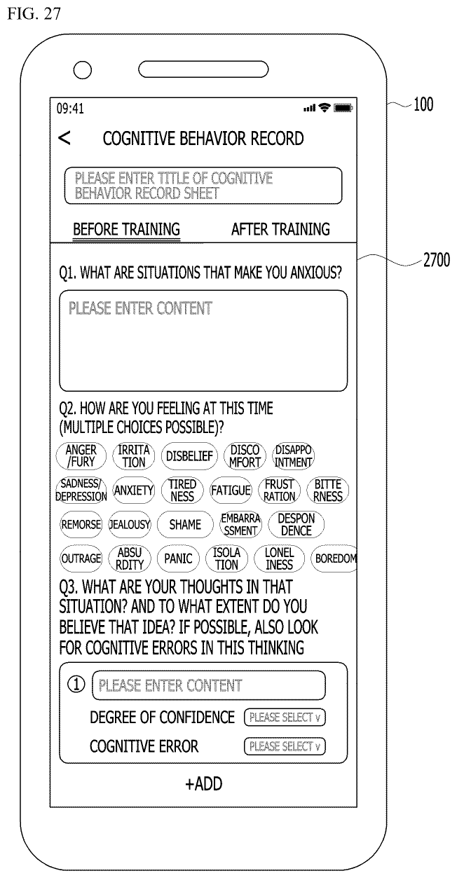

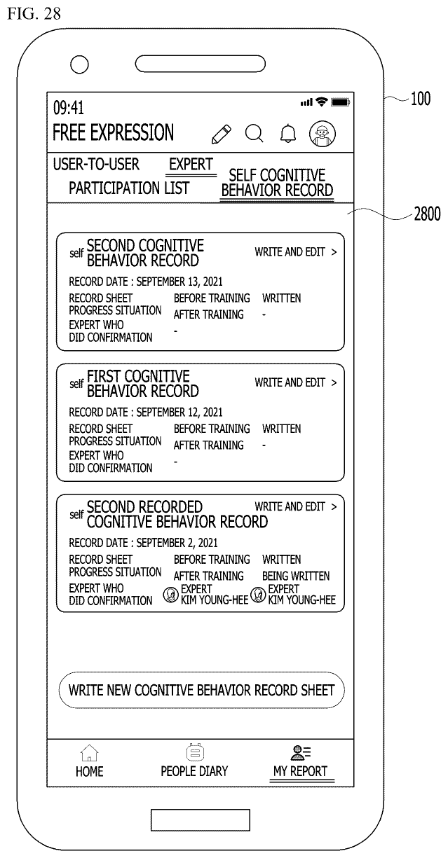

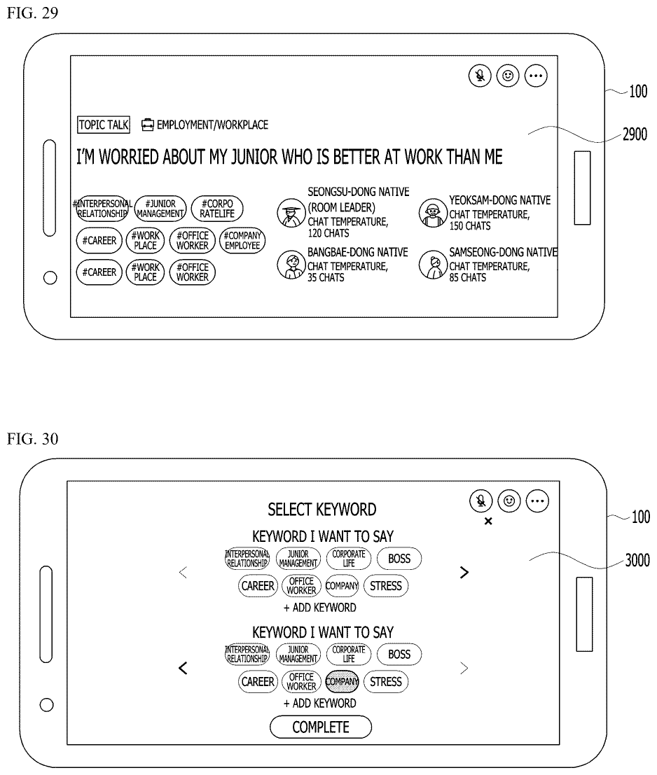

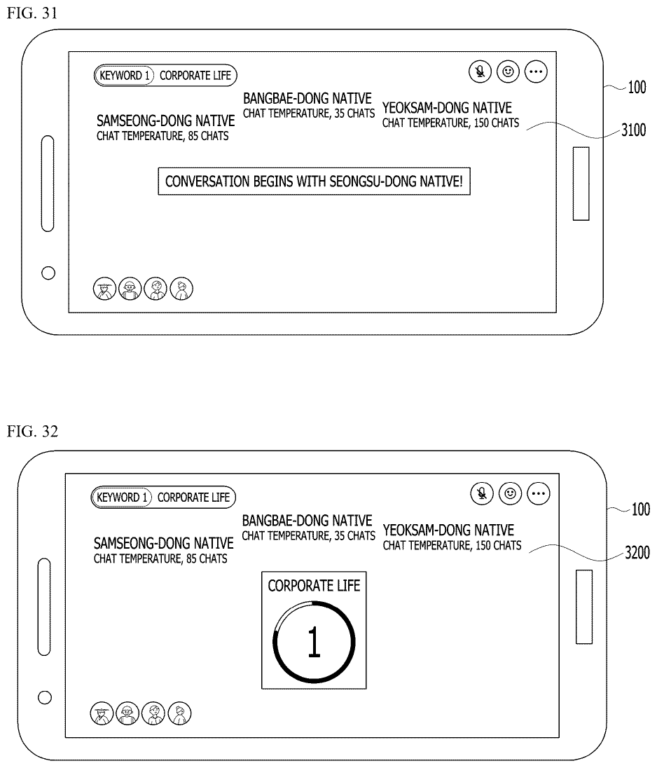

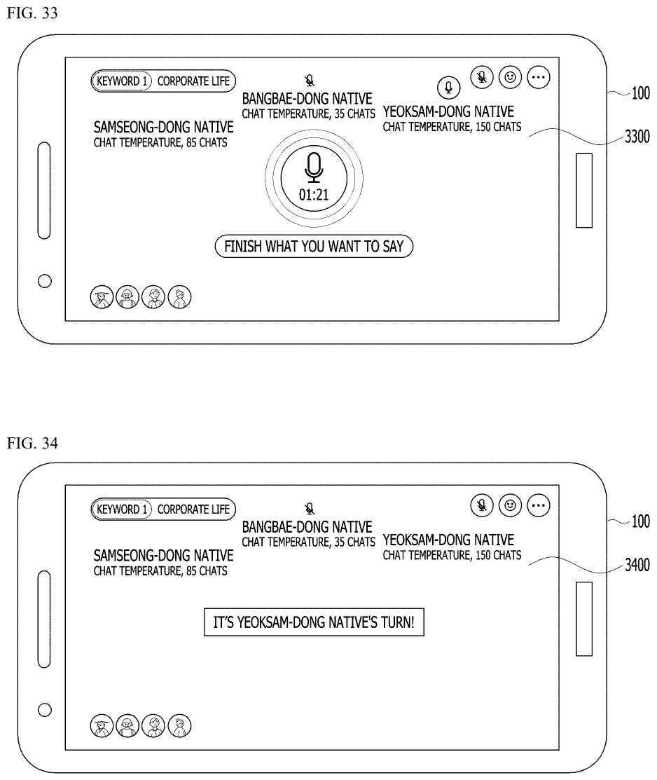

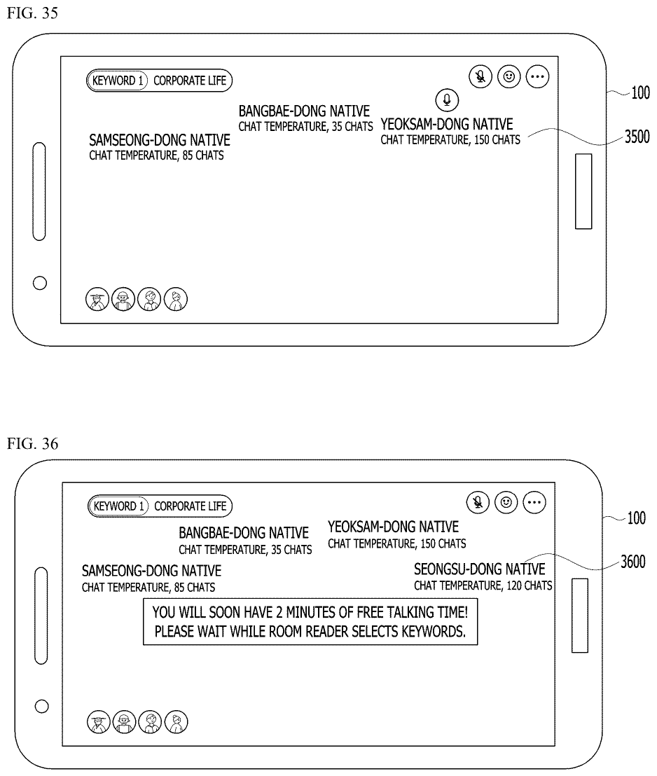

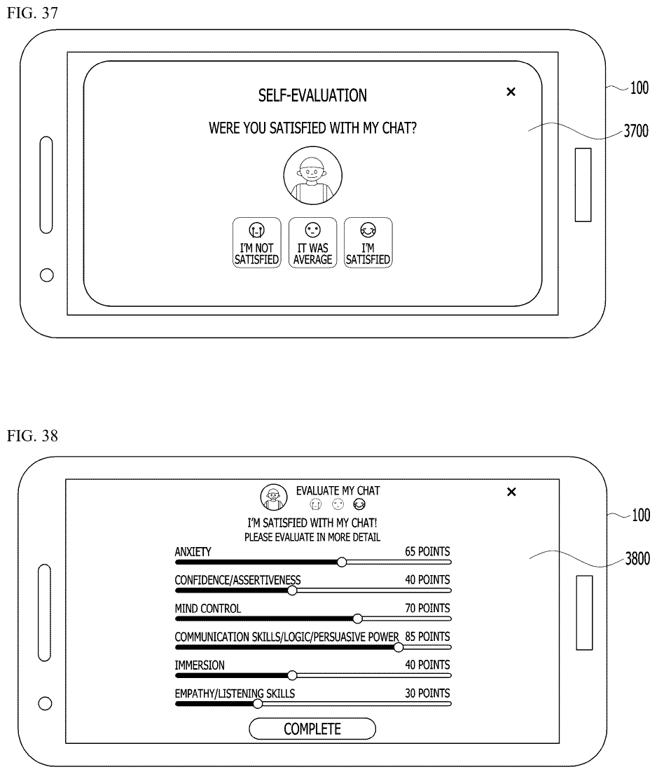

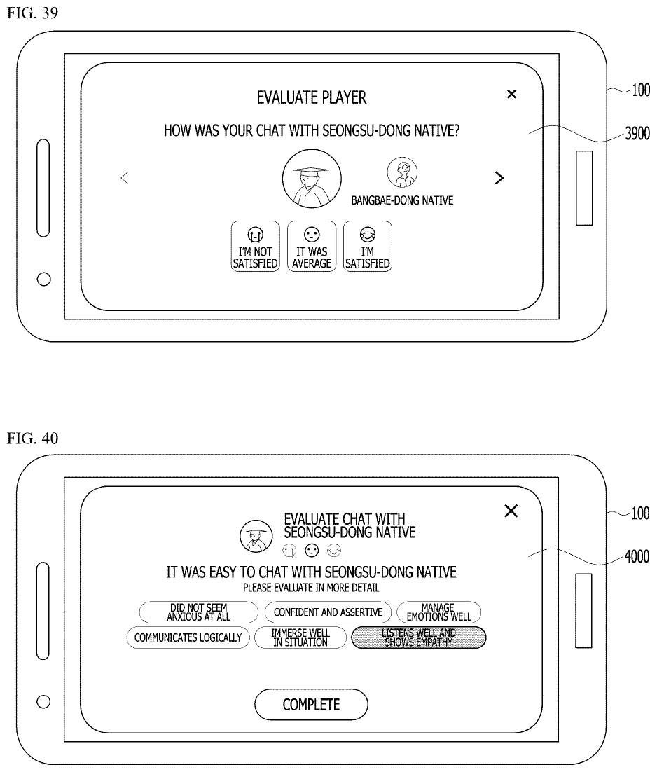

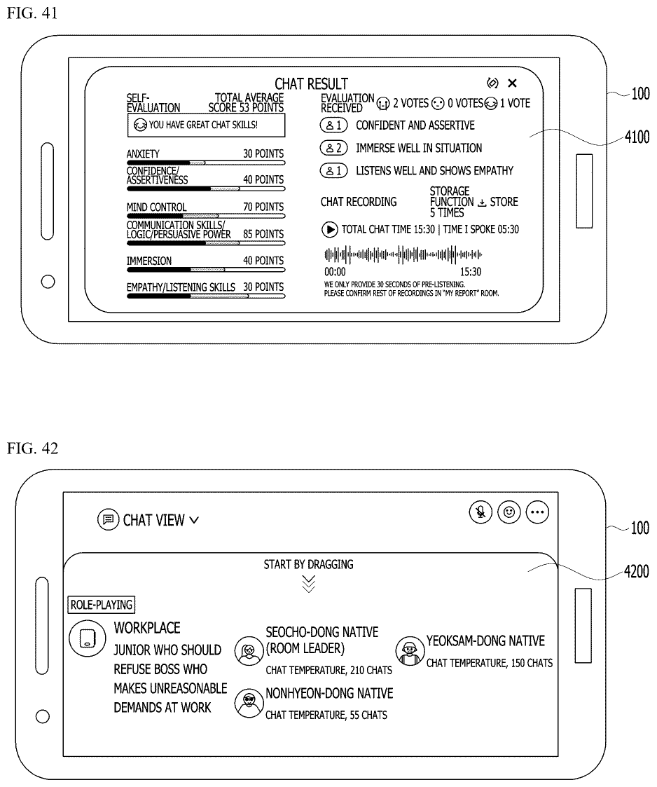

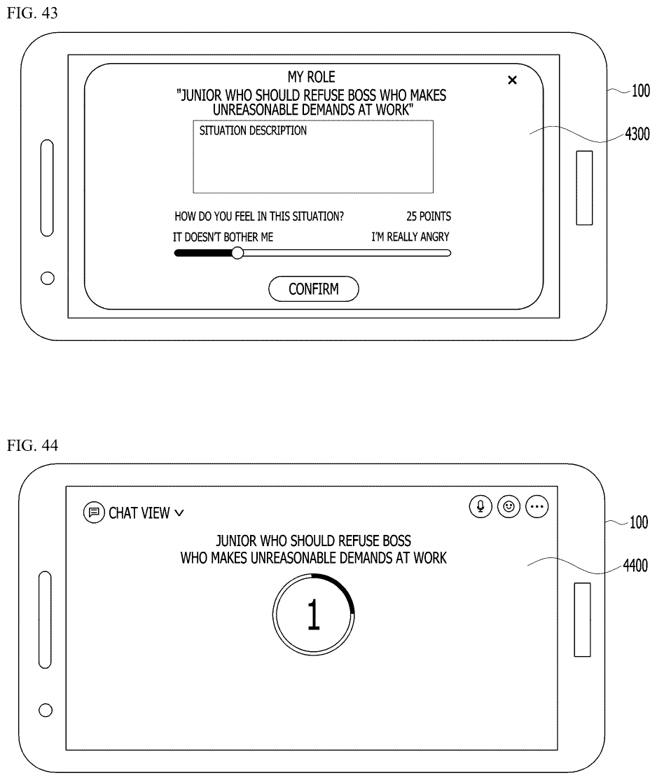

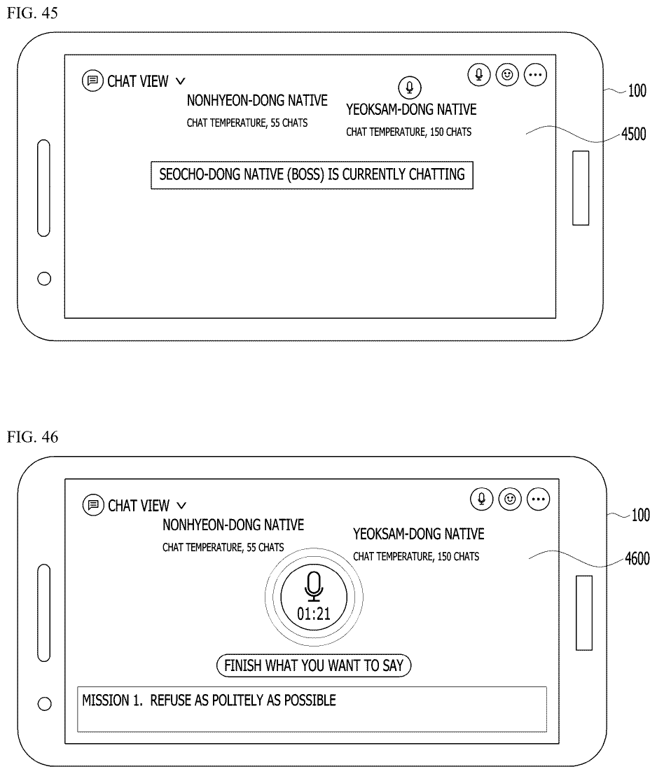

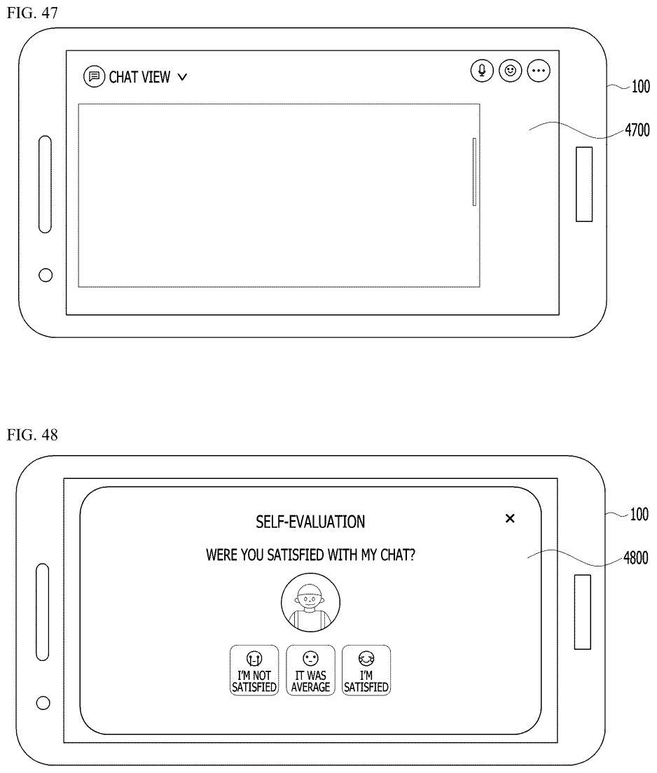

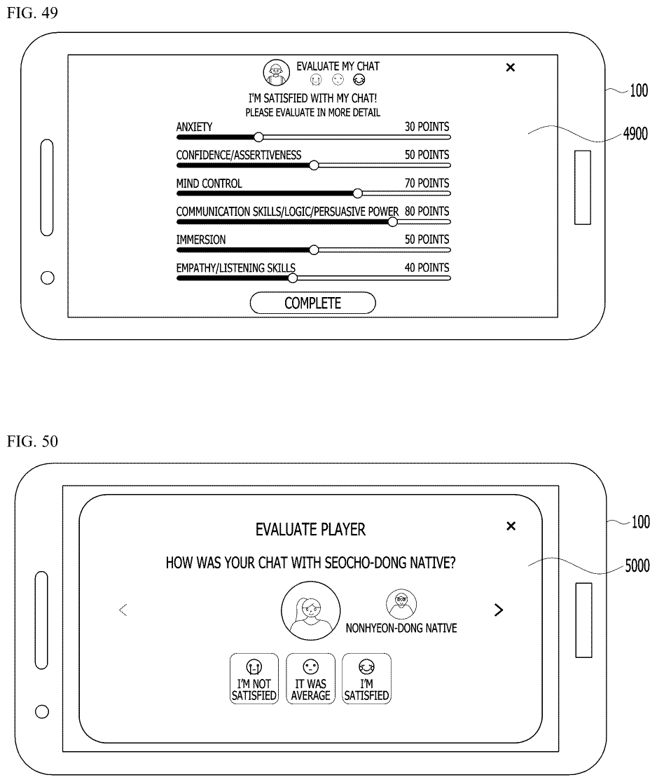

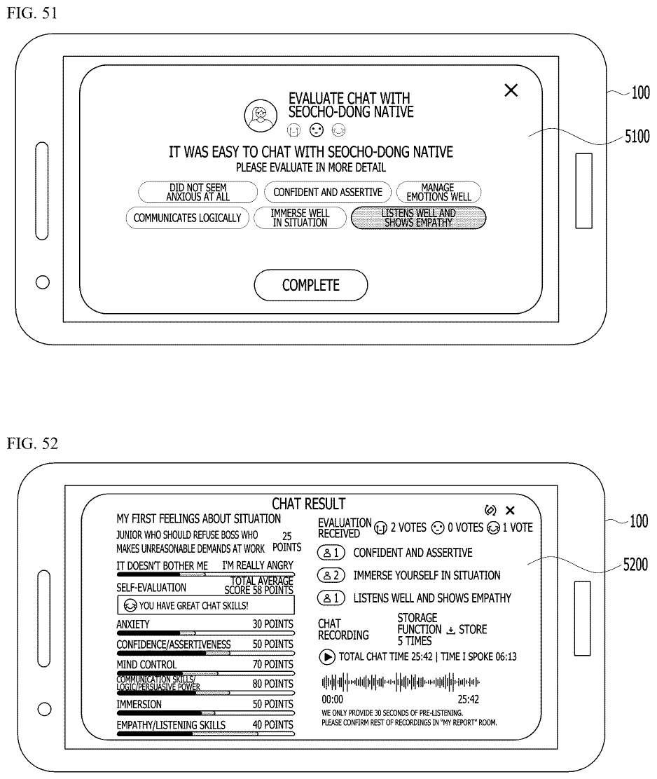

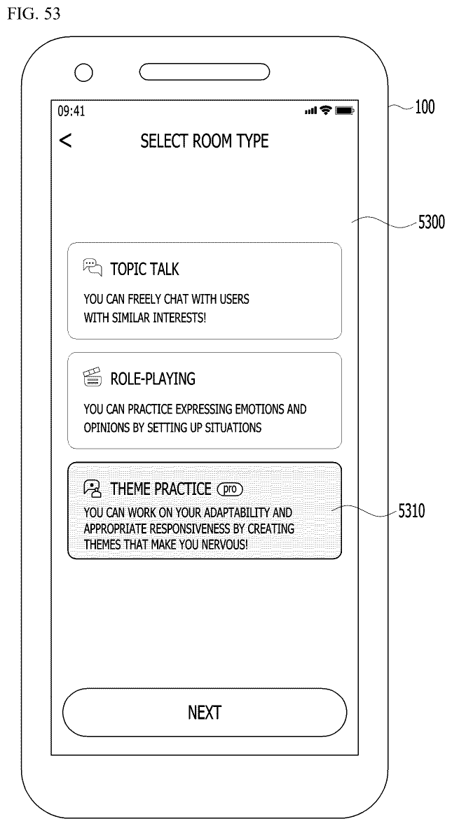

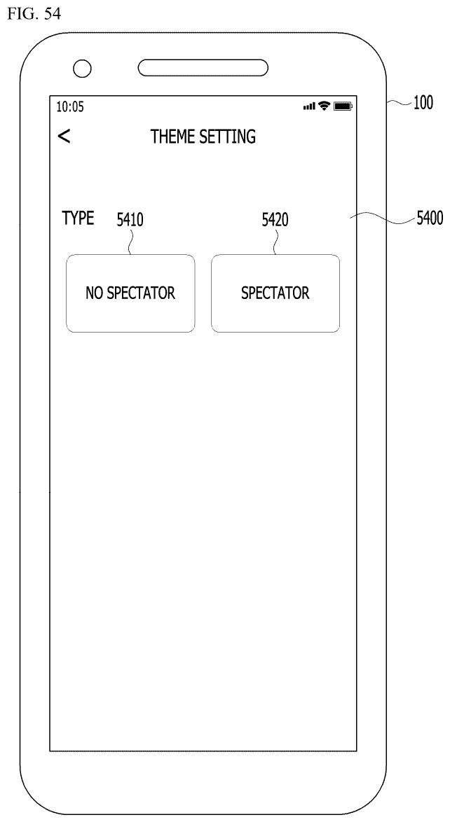

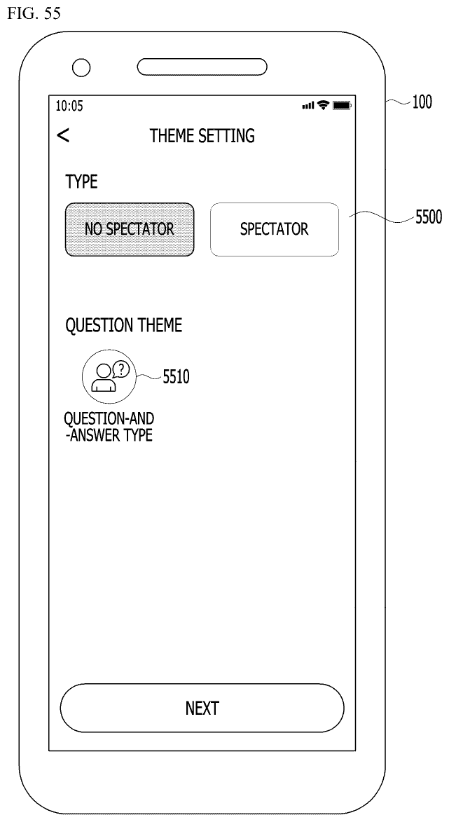

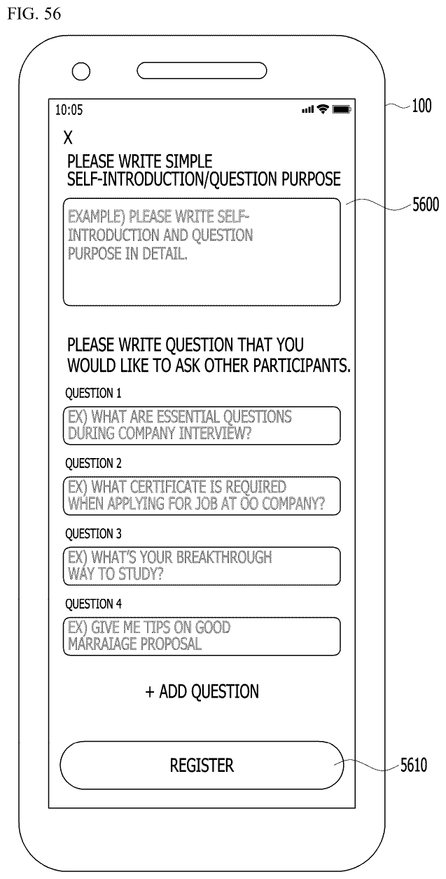

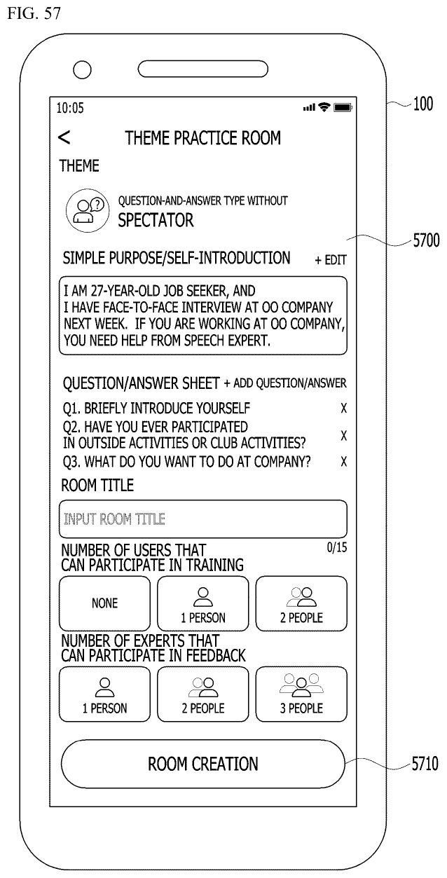

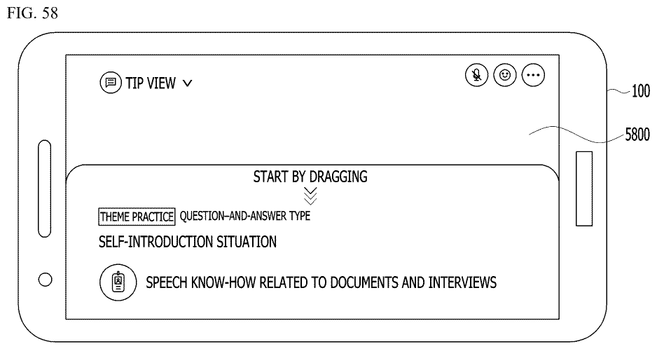

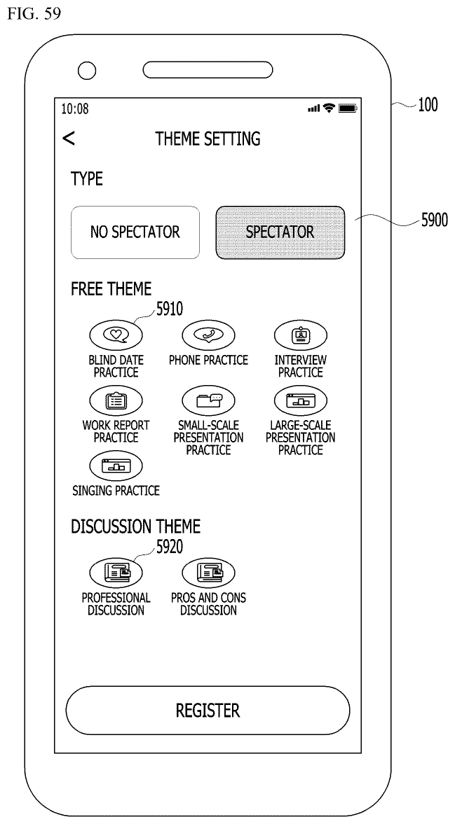

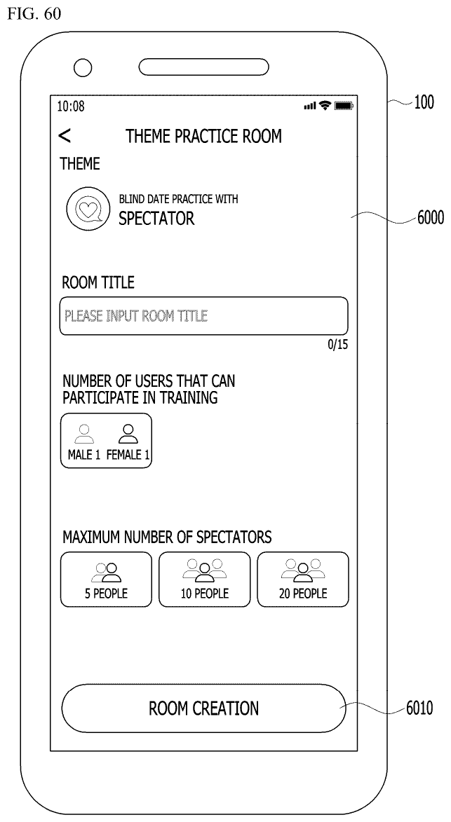

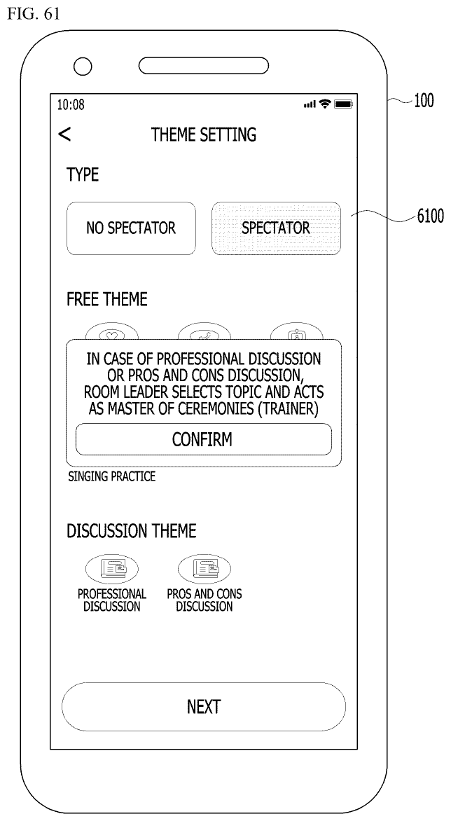

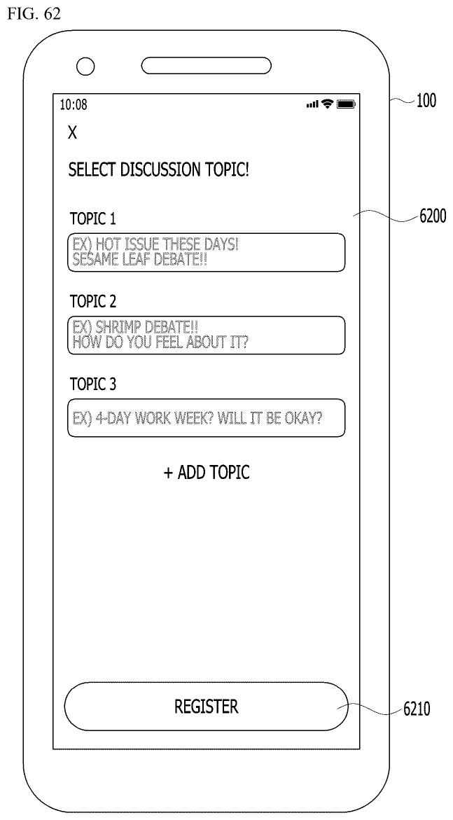

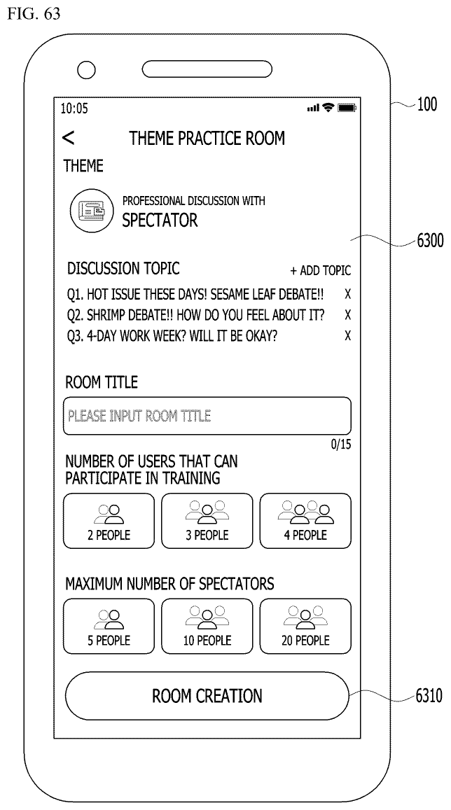

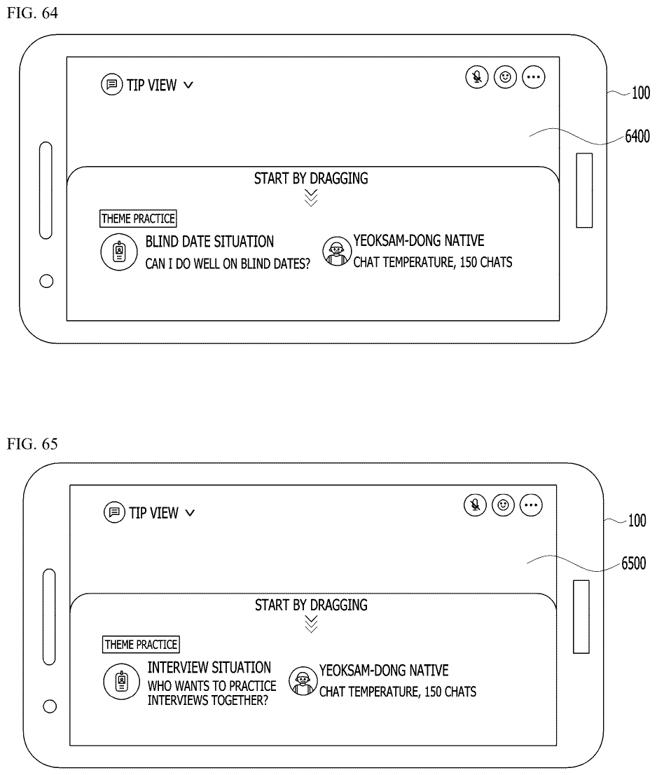

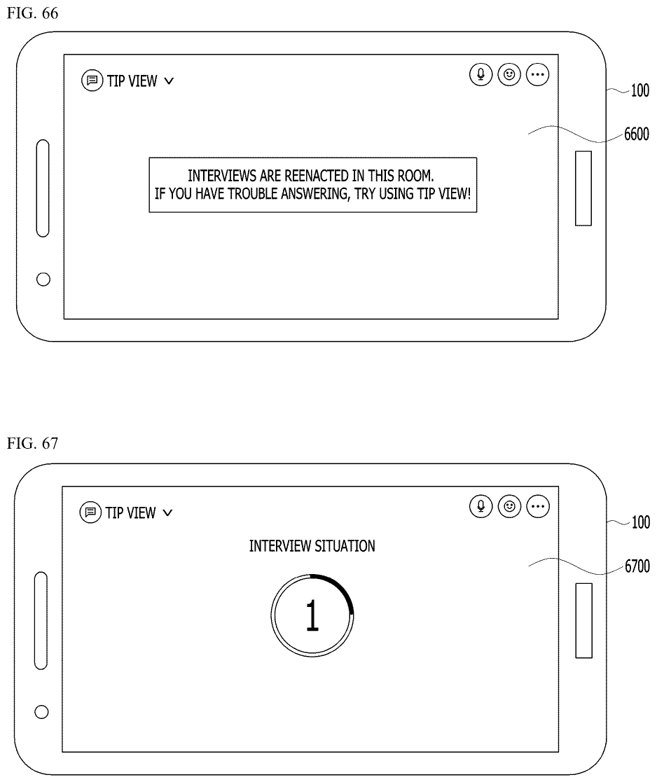

As an example, the first terminal 100 executes the “free expression” app pre-installed on the first terminal, and as illustrated in FIG. 7, displays the “free expression” app execution result screen 700.

In addition, when a people diary menu 710 is selected from the “free expression” app execution result screen 700, as illustrated in FIG. 8, the first terminal displays a people diary screen 800 corresponding to the selected people diary menu 710 (S210).

Thereafter, after a specific date is selected from the people diary screen displayed on the terminal 100 according to the user input (or user selection/touch/control) of the terminal 100, when the preset detailed emotional record menu is selected, the terminal 100 displays a detailed emotional record screen corresponding to the selected detailed emotional record menu. Here, the detailed emotional record screen includes a date selection item for selecting a date, a situation selection item for selecting a specific situation, an emotion selection item for selecting an emotional state felt by a user in relation to the specific situation, a review selection item related to a user's regrets and future resolutions in relation to the specific situation, etc. In addition, examples of the situation selection item include a “Who were you with today?” item (e.g., including family, a date/lover, a friend, work/a coworker, school, a senior/junior, others, etc.) for selecting who on was with, a “What did they do?” item (e.g., including affirmative items such as “They complimented me,” “They empathized with me,” “They comforted me,” “They gave me advice, and “They respected me,” negative items such as “They ignored me, “They were annoyed, “They were upset, “They were angry, and “We argued,” etc.) for selecting what action the counterpart did, a “What action did I take?” item for selecting what action one took oneself (e.g., including affirmative items such as “I were considerate,” “I apologized,” “I acknowledged it,” “I supported them,” and “I helped them,” negative items such as “I was embarrassed,” “I was rambling,” “I was having a hard time,” “I was silent,” “I could not do anything,” and ”I forced a smile,” etc.), etc. In addition, examples of the emotion selection item include a “How did I feel at that time?” item (e.g., including happiness, joy, vitality, confidence, courage, spirit, gratitude, appreciation, passion, inspiration, ecstasy, love, anger/fury, irritation, disbelief, displeasure, disappointment, sadness/depression, anxiety, exhausted, fatigue, frustration, nothingness, etc.) for selecting how the user felt in a corresponding situation, a “Did you express that feeling?” item (e.g., including not expressed, expressed a little, expressed actively, etc.) for selecting whether the user expressed the emotion they felt, a “OO is currently a ‘˜˜˜’ person to me” item (e.g., including affirmative items such as reliable, cute, lovely, and kind, neutral items such as mediocre and average, and negative items such as bad and disgusting, etc.) for selecting how one currently feel about one's counterpart, etc. In addition, the review selection item includes a “Did you feel comfortable talking to that person?” item (e.g., including very uncomfortable, uncomfortable, average, comfortable, very comfortable, etc.) for selecting one's comfort level when talking to a counterpart, a “What do you want to do in the future?” item for selecting one's regrets or future resolutions, a “Please write your own memo to record the situation in more detail at that time,” item, etc.

As an example, after the first date (e.g., Wednesday, Dec. 1, 2021) is selected from the people diary screen 800 displayed on the first terminal, when a preset detailed emotional record menu 810 is selected, as illustrated in FIGS. 9 to 13, a first terminal displays detailed emotional record screens 900, 1000, 1100, 1200, and 1300 corresponding to the selected detailed emotional record menu 810. In this case, the detailed emotional record screens 900, 1000, 1100, 1200, and 1300 are screens corresponding to situation selection items 900 and 1000, emotion selection items 1100 and 1200, and a review selection item 1300, to facilitate user input, and may be divided into corresponding screens.

As another example, after the second date (e.g., Thursday, Dec. 2, 2021) is selected from the people diary screen 800 displayed on the first terminal, when the preset detailed emotional record menu 810 is selected, as illustrated in FIG. 14, the first terminal displays an emotional record status screen 1400 indicating that simple emotional record information already exists on the second date (S220).

Thereafter, the terminal 100 receives detailed emotional record information including a plurality of input values corresponding to the plurality of input items related to the emotion felt by the user in a specific situation on the previously selected specific date (or specific date) according to the user input of the terminal 100 from the detailed emotional record screen displayed on the terminal 100. Here, the detailed emotional record information includes date information, situation information (e.g., including who else was there, other people's behaviors, one's own behaviors, etc.), and emotional information (e.g., including one's emotional state, whether emotions were expressed, one's current emotions, etc.), review information (e.g., level of comfort in chat, future resolutions, memos for oneself, etc.), etc.

In addition, the terminal 100 is linked with the server 200 to map (or match/link) the received detailed emotional record information with the user information of the terminal 100 and stores (or registers/manages) the mapped information in the corresponding server 200.

That is, the terminal 100 transmits the received detailed emotional record information, the emotional record storage request information, the identification information of the terminal 100, etc., to the server 200. Here, the identification information of the terminal 100 includes a mobile directory number (MDN), mobile Internet protocol (IP), mobile media access control (MAC), subscriber identification module (SIM) card unique information, a serial number, etc.

In addition, the server 200 receives the detailed emotional record information, the emotional record storage request information, the identification information of the terminal 100, etc., transmitted from the terminal 100.

In addition, the server 200 confirms (or searches for) the user information corresponding to the received identification information of the terminal 100 among user information (or personal information/member information/expert information) for each piece of identification information of the terminal being managed by the server 200.

In addition, the server 200 maps (or matches/links) the user information corresponding to the identified (or searched) identification information of the corresponding terminal 100 to the received detailed emotional record information, the received identification information of the terminal 100, etc., and stores (or registers/manages) the mapped information in the corresponding server 200.

In addition, the terminal 100 updates and displays the people diary screen related to the corresponding specific date.

That is, the terminal 100 displays the people diary screen to which the detailed emotional record information is applied (or reflected) on the corresponding specific date.

As an example, the first terminal receives first detailed emotional record information including first date information (e.g. Dec. 1, 2021), first situation information (e.g., including “The first person I was with, “The action of the first counterpart confessing,” “My first action sharing my concerns,” etc.), first emotional information (e.g., including “my first emotional state, happy, emotional state intensity 70%,” “my first emotional state, I did not express it,” “my first current emotional state, that friend is a dependable person to me at the moment,” etc.), first review information (e.g., including “level of comfort during first chat, very comfortable,” “I was disappointed that I couldn't confidently say what I wanted to say, which was my first resolution,” “My first resolution for the future is to confidently say what I want to say when I want to say it!” No, first my personal note, friendship with a friend,” etc.), etc., according to the user input in the detailed emotional record screens 1300, 1400, 1500, 1600, and 1700 displayed on the first terminal.

In addition, the first terminal transmits the received first detailed emotional record information, the first emotional record storage request information, the identification information of the first terminal, etc., to the server 200.

In addition, the server 200 receives the first detailed emotional record information, the first emotional record storage request information, the identification information of the first terminal, etc., transmitted from the first terminal.

In addition, the server 200 confirms the first user information corresponding to the received identification information of the first terminal among the user information for each piece of identification information of the terminal being managed by the server 200.

In addition, the server 200 maps and stores the confirmed first user information, first detailed emotional record information, and the identification information of the first expert terminal, etc.

In addition, as illustrated in FIG. 15, the first terminal displays a people diary screen 1500 to which the received first detailed emotional record information is applied (S230).

In addition, after a specific date is selected from the people diary screen displayed on the terminal 100 according to the user input (or user selection/touch/control) of the terminal 100, when the preset simple record menu is selected, the terminal 100 displays a simple emotional record screen corresponding to the selected simple record menu. Here, the simple emotional record screen includes a date selection item for selecting a date, a situation description input item for inputting information about who did what and with whom, a mental state item (e.g., uncomfortable, average, comfortable, other, etc.) for selecting what one's state of mind was, etc.

For example, after the third date (e.g., Friday, Dec. 3, 2021) is selected from the people diary screen 800 displayed on the first terminal, when a preset simple record menu 820 is selected, as illustrated in FIG. 16, a first terminal displays a simple emotional recording screen 1600 corresponding to the selected simple record menu 820 (S240).

Thereafter, the simple emotional record information including a plurality of other input values corresponding to the plurality of other input items related to the emotions felt by the user in a specific situation on the specific date (or specific date) previously selected according to the user input of the terminal 100 on the simple emotional recording screen displayed on the terminal 100 is received. Here, the simple emotional record information includes date information, situation description information (e.g., including who else was there, activity details, etc.), mental state information, etc.

In addition, the terminal 100 is linked with the server 200 to map (or match/link) the received simple emotional record information with the user information of the terminal 100 and stores the mapped information in the corresponding server 200 (or registration/management).

That is, the terminal 100 transmits the received simple emotional record information, the other emotional record storage request information, the identification information of the terminal 100, etc., to the server 200.

In addition, the server 200 receives the simple emotional record information, the other emotional record storage request information, the identification information of the terminal 100, etc., transmitted from the terminal 100.

In addition, the server 200 confirms (or searches for) the user information corresponding to the received identification information of the terminal 100 among user information (or personal information/member information) for each piece of identification information of the terminal being managed by the server 200.

In addition, the server 200 maps (or matches/links) the user information corresponding to the identified (or searched) identification information of the corresponding terminal 100 to the received simple emotional record information, the received identification information of the terminal 100, etc., and stores (or registers/manages) the mapped information in the corresponding server 200.

In addition, the terminal 100 updates and displays the people diary screen related to the specific date.

That is, the terminal 100 displays the people diary screen to which the simple emotional record information is applied (or reflected) on the corresponding specific date.

As an example, second simple emotional record information including third date information (e.g., Dec. 3, 2021), second situation description information (e.g., friend Younghee, etc.), second mental state information (e.g., including “I was comfortable,” etc.), etc., is received according to the user input from the simple emotional record screen 2000 displayed on the first terminal.

In addition, the first terminal transmits the received second simple emotional record information, second emotional record storage request information, the identification information of the first terminal, etc., to the server 200.

In addition, the server 200 receives the second simple emotional record information, the second emotional record storage request information, the identification information of the first terminal, etc., transmitted from the first terminal.

In addition, the server 200 confirms the first user information corresponding to the received identification information of the first terminal among the user information for each piece of identification information of the terminal being managed by the server 200.

In addition, the server 200 maps and stores the confirmed first user information, the second simple emotional record information, and the identification information of the first expert terminal, etc.

In addition, the first terminal displays the people diary screen to which the received second simple emotional record information is applied (S250).

In addition, when a preset calendar menu is selected from the people diary screen displayed on the terminal 100, the terminal 100 displays a calendar screen including an emotional record state by date corresponding to the selected calendar menu to display an emotional record state by date. Here, when the emotional record information (including detailed emotional record information, simple emotional record information, etc.) has been stored on the specific date, the calendar screen includes emotional record states such as images (or emoticons/avatars) representing feeling comfortable, normal, uncomfortable, etc., corresponding to mental state information, which is some of the information within the corresponding emotional record information on the corresponding specific date.

As an example, when a calendar menu 830 is selected from the people diary screen 800, as illustrated in FIG. 17, a first terminal displays a calendar screen 1700 including the emotional record state for each date in December 2021 corresponding to the selected calendar menu 830 (S260).

In addition, when a preset “my report” menu is selected from the app execution result screen displayed on the terminal 100, to provide the analysis results and participation list related to the chat room in which the user of the terminal 100 participates or provide the participation list and cognitive behavior therapy record related to the chat room in which the user of the terminal 100 participates with the expert, the terminal 100 displays the “my report” screen corresponding to the selected “my report” menu. Here, the “my report” screen includes an edit item for screen editing, a search item for searching for the chat room corresponding to the search word, a notification item for confirming notices or notification messages, etc., a “my page” item (or setting item) for setting user information or dedicated app functions, etc., a user-to-user item for providing analysis results based on the performance of functions such as topic talk, role-playing, and theme practice in relation to the user of the terminal or a participation list related to the chat list in which the user participates, an expert interaction item for providing a cognitive behavior therapy record, which is a list or feedback from the expert performing the psychological counseling function, the speech lesson function, or the mentoring function with an expert in relation to a specific topic or situation, a content display item (or content display area) for displaying the detailed information according to the user-to-user item or expert interaction item selection, etc. In addition, the user-to-user item (or user-to-user tab) includes an analysis result item for providing analysis results according to the performance of functions such as topic talk, role-playing, and theme practice in relation to the user of the terminal 100, a participation list item for providing a participation list related to the chat list in which the user participates, etc. In addition, the expert interaction item (or expert interaction tab) includes a participation list item for providing a list of people who have performed a psychological counseling function, a speech lesson function, or a mentoring function with an expert in relation to a specific topic or situation, a cognitive behavior therapy record item for providing expert feedback based on performance of a training function on appropriate chat room methods in relation to the specific topic or situation, etc.

In addition, when an analysis result item within a preset user-to-user item is selected from the “my report” screen displayed on the terminal 100, the terminal 100 displays the detailed information corresponding to the selected analysis result menu in the content display area to provide analysis results according to the performance of chat functions such as topic talk, role-playing, and theme practice in relation to the user of the terminal 100. In this case, when the preset “my report” menu is selected from the app execution result screen displayed on the corresponding terminal 100, the terminal 100 may display, by default, an analysis result screen (or the detailed information) corresponding to the analysis result item within the item among users set as default in response to the selected “my report” menu in the content display area. Here, the analysis result item (or the analysis result screen/detailed information) includes a sort item for sorting functions related to all, topic talk, role-playing, theme practice, etc., a “number of times you've participated in the chat room” display item for displaying the number of times the user has participated in the chat room within a preset period, a period display item for displaying the corresponding preset period, a “number of times you've participated in the chat room by category” display item for displaying the number of times the user has participated in the chat room by category, a “free expression” index display item for displaying the “free expression” index calculated in relation to the user, an anxiety index display item for displaying the anxiety index calculated in relation to the user, a detailed score view item for displaying additional detailed information in relation to the “free expression” index or the anxiety index, a relative evaluation display item for displaying information on oneself as evaluated by other users who performed the chat function together, a “my ‘free expression’ score aspect” display item for displaying the “free expression” score aspect, a “my anxiety score pattern” display item for displaying the user's anxiety score pattern, a “my confidence/assertiveness, mind control, communication skills/logic/persuasive power, immersion, empathy/listening skills score aspect” display item for displaying the user's confidence/assertiveness, mind control, communication skills/logic/persuasive power, immersion, empathy/listening skills score aspect, a “topic I have practiced a lot” display item for displaying a topic that the user has practiced a lot in relation to the topic talk, a “keyword that I have practiced a lot” display item for displaying keywords that the user has practiced a lot in relation to the topic talk, a “target I've practiced a lot” display item for displaying what the user has practiced a lot in relation to role-playing, a “situation I've practiced a lot” display item for displaying situations in which the user has practiced a lot in relation to role-playing, a “What emotions am I sensitive to?” display item for displaying what emotions the user is sensitive to in relation to role-playing, a “Which experts I have practiced with a lot?” display item for a displaying indicating with which experts the user has practiced a lot in relation to the theme, a “theme I practiced a lot” display item for displaying a theme that the user has practiced a lot in relation to theme practice, etc. Here, the “free expression” index display item includes an overall rating display item, a “free expression” index overall average display item, a satisfaction graph display item for overall average, a “free expression” index change graph display item by day of the week, a score display item for each item (e.g., including confidence/assertiveness, mind control, communication skills/logic/persuasive power, immersion, empathy/listening skills, etc.), a graph display item of a score change for each item by day of the week, an exponential distribution display item for each topic and situation, a detailed score view item, etc. In addition, the anxiety index display item includes a total rating display item, an anxiety index overall average display item, an anxiety graph display item for the overall average, a graph display item of an anxiety index change by day of the week, etc. In this case, the “free expression” index can be calculated by adding up the average score of the user's self-evaluation after performing the chat function in the chat room (e.g., including the topic talk chat room, the role-playing chat room, the theme practice chat room, etc.) in which the user participates, in relation to one or more chat rooms in which the user of the corresponding terminal 100 participates for a preset period or all chat rooms in which the user of the corresponding terminal 100 participates, and results (or evaluation received) (e.g., +1 for affirmative evaluation/satisfied, −1 for negative evaluation/not great) of evaluation of the user by the counterpart in the chat room in which the user participates. In addition, the anxiety index represents the average value of the anxiety self-evaluated by the user after performing the chat function in the chat room (e.g., including the topic talk chat room, the role-playing chat room, the theme practice chat room, etc.) in which the user participates in relation to one or more chat rooms in which the user of the corresponding terminal 100 participates for a preset period or all chat rooms in which the user of the corresponding terminal 100 participates. In addition, examples of the relative evaluation display item include the reason for receiving a good evaluation, the reason for receiving a bad evaluation, and the satisfaction of chat evaluated by others. In addition, examples of the “my ‘free expression’ score aspect” display item include a “How many people am I with when my free expression score gets high?” item, a “What age group of people am I talking with when I have a high score in ‘free expression’?” item, a “What MBTI type am I talking with when I have a high score in ‘free expression’?” item, a “What gender is the person I'm talking with when I have a high score in ‘free expression’?” item, a “When I talk in a certain place, do I have a high score in free expression?” item, etc. In addition, the “my anxiety score aspect” display item includes a “How many people am I with when my anxiety score gets high?” item, a “What age group of people am I talking with when I have a high score in anxiety?” item, a “What MBTI type am I talking with when I have a high score in anxiety?” item, a “What gender is the person I'm talking with when I have a high score in anxiety?” item, a “When I talk in a certain place, do I have a high score in anxiety?” item, etc. In addition, examples of the “topic that I have practiced a lot” display item include an interpersonal relationship item, a daily life item, an employment/workplace item, a knowledge sharing item, a well-being item, other items, etc. In addition, examples of the “keyword that I have practiced a lot” display item include a boss item, a soccer item, a job preparation item, a presentation preparation item, a health item, other items, etc. In addition, examples of the “target that I have practiced a lot” display item include a husband item, a boss item, a coworker item, a chairman item, a stranger item, other items, etc. In addition, examples of the “situation that I have practiced a lot” display item include a rejection expression item, an affection expression item, an opinion expression item, a gratitude/sorry expression item, a conflict item, other items, etc. In addition, examples of the “What emotions am I sensitive to?” display item include a joy/pleasure item, an empathy item, an appreciation item, a sorry item, a grief/hurt item, an anger/annoyance/rage item, etc. In addition, examples of the “Which experts I have practiced with a lot?” display item include a psychological counselor item, a speech trainer item, a mentor item, etc. In addition, examples of the “theme that I practiced a lot” display item include a blind date item, a phone item, an interview item, a business report item, a small-scale presentation item, a large-scale presentation item, a professional discussion item, a pros and cons discussion item, etc.

As an example, when a “my report” menu 720 is selected from the “free expression” app execution result screen 700, as illustrated in FIG. 18, the first terminal displays a “my report” screen 1800 including an analysis result screen corresponding to the analysis result item in the user-to-user item set by default corresponding to the selected my report menu 720 (S270).

Hereinafter, when a preset detailed score view item is selected from the detailed information included in the content display area displayed on the terminal 100, the terminal 100 is linked with the server 200 to display detailed score information corresponding to the selected detailed score view item. Here, the detailed score information (or detailed score screen) includes the sort items for sorting functions related to all, topic talk, role-playing, theme practice, etc., the “number of times you've participated in the chat room” display item, the period display item for displaying the corresponding preset period, the “number of times you've participated in the chat room for each category” display item, the “free expression” index display item, the anxiety index display item, the relative evaluation display item, the “my ‘free expression’ score aspect” display item, the “my anxiety score aspect” display item, the “my confidence/assertiveness, mind control, communication skills/logic/persuasive power, immersion, empathy/listening skills score pattern” display item, the “topic I have practiced a lot” display item, the “keyword that I have practiced a lot” display item, the “target that I have practiced a lot” display item, the “situation that I have practiced a lot” display item, the “Which emotions I am sensitive to?” display item, the “Which experts have I practiced with a lot?” display item, the “theme which I have practiced a lot” display item, etc. In this case, when the detailed score view item related to the “free expression” index is selected, the terminal 100 may display one or more pieces of information related to the “free expression” index among the information included in the detailed score information, and when the detailed score view item related to the anxiety index is selected, the terminal 100 may mainly display one or more other pieces of information related to the anxiety index among the information included in the detailed score information.

That is, when a preset detailed score view item is selected from the detailed information included in the content display area displayed on the terminal 100, the terminal 100 is linked with the server 200 to display the detailed score information corresponding to the selected detailed score view item instead of the “free expression” index display item or anxiety index display item displayed previously.

For example, when a detailed score view item 1810 is selected from the “my report” screen 1800 displayed on the first terminal, as illustrated in FIG. 19, the first terminal displays a detailed score screen 1900 corresponding to the selected detailed score view item 1810 provided from the server 200 (S280).

In addition, when the participation list item in the preset user-to-user item is selected from the my report screen displayed on the terminal 100, the terminal 100 displays a participation list screen corresponding to the selected participation list item to provide a participation list related to the chat list in which the user of the terminal 100 participates. Here, the participation list screen includes a sort item for sorting functions related to all, topic talk, role-playing, theme practice, etc., a change information display item for providing information on changes in self-evaluation results or other's evaluation results compared to a preset period (or last week), a “number of times you've participated in the chat room” display item for displaying the number of times the user has participated in the chat room within a preset period, an information display item for each chat participation case for providing information related to topic talk, role-playing, theme practice, etc., in which the user participates, etc. In this case, the information display item for each chat participation includes a date information display item of participation in the chat room, a detail view item for displaying the detailed information, a chat room type (or chat room category) display item (e.g., including topic talk, role-playing, theme practice, etc.) for displaying the type of chat room in which the user participates, a chat room topic display item for displaying the topic of the chat room, a chat room name display item for displaying the room title (or chat room name) of the chat room, a keyword display item for displaying the main keywords/hashtags related to the corresponding chat room, a self-evaluation information display item for displaying the self-evaluation results, a received evaluation information display item for displaying the evaluation information received from other users participating in the corresponding chat room, a chat recording display item for the re-listening function of chat content from the chat room, etc.

In addition, when a specific participation list (or a detail view item displayed on one side of the corresponding specific participation list) is selected from one or more participation lists displayed in the information display items for each chat participation in the participation list screen displayed on the terminal 100, the terminal 100 is linked with the server 200 to display the detailed view information related to the selected specific participation list. Here, the detailed view information includes a date information display item of participation in the chat room corresponding to the selected specific participation list, a chat room type (or chat room category) display item for displaying the type of chat room in which the user participates (e.g., including a topic talk chat room, a role-playing chat room, a theme practice chat room, etc.), a chat room topic display item for displaying the topic of the chat room, a chat room name display item for displaying the room title of the chat room, a keyword display item for displaying main keywords/hashtags related to the chat room, a chat participant information display item for displaying information (e.g., including nicknames) of other users participating in the corresponding chat room, a self-evaluation result display item (or self-evaluation information display item) for displaying self-evaluation results, a received evaluation result display item (or a received evaluation information display item) for displaying evaluation results received from other users participating in the chat room, a chat recording display item for re-listening to chat content from the chat room, etc.

In addition, when a participation list item 1820 in the user-to-user item is selected from the my report screen 1800 displayed on the first terminal, as illustrated in FIG. 20, the first terminal displays a participation list screen 2000 corresponding to the selected participation list item 1820.

In addition, when a detail view item 2010 related to “I'm worried about my junior who is better at work than me” from Sep. 1, 2021 in the participation list screen 2000 displayed on the first terminal is selected, as illustrated in FIG. 21, the first terminal displays detailed item information 2100 corresponding to the selected detail view item 2010 provided from the server 200 (S290).

In addition, when the participation list item (or other participation list items) in the preset expert interaction item is selected from the “my report” screen displayed on the terminal 100, the terminal 100 displays other participation list screens corresponding to the selected participation list item to provide other participation lists in which the user of the terminal 100 performs a counseling function, a speech lesson function, a mentoring function, etc., with an expert. Here, the other participation list screens include other sort items for sorting functions related to all, psychological counseling, speech, mentoring, etc., other change information display items for providing information on changes in the number of counseling sessions or the number of chat training sessions with an expert or changes of a cognitive behavior therapy record compared to a preset period (or last week), other “number of times you've participated in the chat room” display items for displaying the number of times the user has participated in the chat room within a preset period, an information display item for each expert chat participation case for providing information related to psychological counseling, speech, mentoring, etc. in which the user participates, etc. In this case, examples of the information display item for each expert chat participation case include other chat room type (or chat room category) display items for displaying the type of chat room in which the user participates (e.g., including psychological counseling, speech, mentoring, etc.), another chat room topic display item for displaying the topic of the chat room, a detail view item for displaying detailed information, an expert information display item for displaying an expert's photo and name, a counseling date display item for displaying a counseling date, other keyword display items for displaying main keywords/hashtags related to the corresponding chat room, an expert feedback item for providing expert feedback, a cognitive behavior therapy record sheet item for providing a cognitive behavior therapy record sheet, a written questionnaire item for providing questionnaire content written by a user, a chat room training display item for displaying chat room training content, etc.

In addition, when other specific participation lists (or detail view item displayed on one side of the corresponding other specific participation lists) are selected from one or more other participation lists being displayed in the information display items for each expert participation in other participation list screens displayed on the terminal 100, the terminal 100 is linked with the server 200 to display the detailed information related to the selected other specific participation lists. Here, the detailed information (or detailed counseling result item screen) includes other chat room type (or chat room categories) display items for displaying the types of chat rooms (e.g., including psychological counseling, speech, mentoring, etc.) in relation to the other specific participation lists, other chat room topic display items for displaying the topic of the chat room, an expert information display item for displaying the expert's photo and name, a counseling date display item for displaying the counseling date, other keyword display items for displaying the main keywords/hashtags related to the corresponding chat room, an expert feedback display item for displaying the expert feedback, a cognitive behavior therapy record sheet item for providing the cognitive behavior therapy record sheet before and after training, a questionnaire item written during counseling for providing the type and results of the questionnaire written by the user, a chat room participation record display item for providing the chat room participation record, etc. In this case, the questionnaire item written during counseling may include various types of tests (or questionnaires) such as a self-esteem self-diagnosis test, a communication type test, an anxiety test, etc. In addition, examples of the chat room participation record display item include a date information display item of participation in the chat room, an expert state item for displaying whether the expert is participating in training or observing the expert, a chat room type (or chat room category) display item for displaying the type of chat rooms (e.g., including a topic talk chat room, a role-playing chat room, a theme practice chat room, etc.) in which the user participates, a chat room topic display item for displaying the topic of the chat room, a chat room name display item for displaying the room title of the chat room, a chat participant information display item for displaying information (e.g., including nicknames) of other users participating in the corresponding chat room, a self-evaluation result display item (or self-evaluation information display item) for displaying the self-evaluation results, a received evaluation result display item (or received evaluation information display item) for displaying the evaluation results received from other users participating in the corresponding chat room, a chat recording display item for the re-listening function of the chat content from the chat room, etc.

As an example, after the expert interaction item is selected from the my report screen 1800 displayed on the first terminal, when the participation list item within the corresponding expert interaction item is selected, as illustrated in FIG. 22, the first terminal displays other participation list screens 2200 corresponding to the participation list item within the selected expert interaction item.

In addition, when a detail view item 2210 related to “session 4, overcoming anxiety” performed on Sep. 12, 2021 in the other participation list screens 2200 displayed on the first terminal is selected, as illustrated in FIG. 23, the first terminal displays detailed information 2300 corresponding to the selected detail view item 2210 provided from the server 200 (S300).

In addition, when the cognitive behavior therapy record item in the preset expert interaction item is selected from the “my report” screen displayed on the terminal 100, to provide expert feedback according to the user of the terminal 100 performing a training function on an appropriate chat room type in relation to a specific topic or situation, the terminal 100 displays the cognitive behavior therapy record screen corresponding to the selected cognitive behavior therapy record item. Here, the cognitive behavior therapy record screen (or cognitive behavior therapy record sheet screen) includes a cognitive behavior therapy record information display item for each chat participation for writing (or providing) a cognitive behavior therapy record before and after training in relation to a specific topic or situation in which the user participates, a new cognitive behavior therapy record sheet writing item for writing a new cognitive behavior therapy record sheet, etc. In addition, examples of the cognitive behavior therapy record information display item for each chat participation include a cognitive behavior therapy record sheet title display item for displaying a title, a writing and modification item for writing or editing a non-written cognitive behavior therapy record, a result view item for displaying the written cognitive behavior therapy record results, a record date display item for displaying a date the cognitive behavior therapy record is written, a record sheet progress display item for displaying record sheet progress before/after training, a confirmed expert display item for displaying an expert who has confirmed a cognitive behavior therapy record sheet written by a user, etc.

In addition, when the writing and modification item displayed on one side of the specific cognitive behavior therapy record is selected from among one or more cognitive behavior therapy record items displayed in the cognitive behavior therapy record information display item for each chat participation in the cognitive behavior therapy record screen displayed on the terminal 100, the terminal 100 is linked with the server 200 to display the cognitive behavior therapy record input screen corresponding to the selected writing and modification item to edit the cognitive behavior therapy record information related to the selected specific cognitive behavior therapy record. Here, in the case of the pre-training, the cognitive behavior therapy record input screen includes the “What are situations that make you anxious?” item, the “How are you feeling at this time” item (e.g., including anger/fury, irritation, disbelief, discomfort, disappointment, sadness/depression, anxiety, tiredness, fatigue, frustration, bitterness, remorse, jealousy, shame, embarrassment, despondence, outrage, panic, isolation, loneliness, boredom, etc.), the “What are your thoughts in that situation?” item (e.g., including a confidence level, a cognitive error, etc.), the “please correct my wrong thoughts” item (or reasonable response finding item), the behavioral goal setting item, etc. In this case, the types of cognitive errors include black and white (or black and white logic), divination (or fortune teller/catastrophe), ignoring (or ignoring advantages), inference (or emotional inference), name (or naming), mind reading (or mind reading technique), filtering (or mental filtering), obsession (or obsessive burden), uselessness (or useless thoughts), etc. In addition, the cognitive behavior therapy record input screen after training includes a “Please display any area that has improved slightly through training” item (e.g., an improvement-related item, an anxiety-related item, etc.), a “What actually happened related to an area I was worried about before training?” item, a “Please re-evaluate your thoughts before training” item, a “how I feel now” selection item (e.g. happiness, joy, vitality, confidence, courage, spirit, gratitude, appreciation, passion, inspiration, ecstasy, love, anger/rage, irritation, disbelief, displeasure, disappointment, sadness/depression, anxiety, tiredness, fatigue, frustration, etc.), a “What did you learn?” item, etc.

In addition, the terminal 100 receives a plurality of input values corresponding to the plurality of input items included in the cognitive behavior therapy record input screen displayed on the terminal 100 according to the user input. Here, the plurality of input values include the input values before training (e.g., including “What are situations that make you anxious?” content, “How are you feeling at this time” content, “What are your thoughts in that situation?” content, “Please correct my wrong thoughts” content, behavioral goal setting content etc.), the input values after training (e.g., “Please display any area that has improved slightly through training” content, “What actually happened related to an area I was worried about before training?” content, “Please re-evaluate your thoughts before training” content, “how I feel now” content, “What did you learn?” content, etc.).

In addition, the terminal 100 is linked with the server 200 to apply the plurality of received input values to the user information of the terminal 100 and the cognitive behavior therapy record information related to the specific cognitive behavior therapy record case and display the cognitive behavior therapy record screen in the state in which the plurality of input values are applied (or updated).

In addition, when the item for writing a new cognitive behavior therapy record sheet in the cognitive behavior therapy record screen displayed on the terminal 100 is selected, the terminal 100 is linked with the server 200 to display a new cognitive behavior therapy record sheet writing screen corresponding to the selected new cognitive behavior therapy record sheet writing item. Here, the new cognitive behavior therapy record sheet creation screen includes a cognitive behavior therapy record sheet title input item for inputting the cognitive behavior therapy record sheet title, a pre-training or post-training item for selecting pre/post-training, a pre-training input item (e.g., including “What are situations that make you anxious?” “How are you feeling at this time?” “What are your thoughts in that situation?” “Please correct my wrong thoughts,” “Please set action goal,” etc.), a post-training input item (e.g., including “Please display any area that has improved slightly through training,” “What actually happened in relation to an area I was worried about before training?” “Please re-evaluate your thoughts before training,” “How do you feel now?” “What have you learned?” etc.) etc.

In addition, the terminal 100 receives a plurality of other input values corresponding to the plurality of other input items included in the new cognitive behavior therapy record input screen displayed on the terminal 100 according to the user input. Here, the plurality of other input values include the cognitive behavior therapy record sheet title, the input values before training (e.g., including “What are situations that make you anxious?” content, “How are you feeling at this time” content, “What are your thoughts in that situation?” content, “Please correct my wrong thoughts” content, behavioral goal setting content, etc.), the input values after training (e.g., “Please display any area that has improved slightly through training” content, “What actually happened related to an area I was worried about before training” content, “Please re-evaluate your thoughts before training” content, “how I feel now” content, “What did you learn?” content, etc.).

In addition, the terminal 100 is linked with the server 200 to generate the new cognitive behavior therapy record related to the user (or user information) of the terminal 100 based on the plurality of received different input values, apply the plurality of received different input values to the cognitive behavior therapy record information related to the generated new cognitive behavior therapy record case, and display the cognitive behavior therapy record screen in the state in which the plurality of different input values are applied.

As an example, after the expert interaction item is selected from the “my report” screen 1800 displayed on the first terminal, when the cognitive behavior therapy record item within the corresponding expert interaction item is selected, as illustrated in FIG. 24, the first terminal displays a cognitive behavior therapy record screen 2400 corresponding to the cognitive behavior therapy record item within the selected expert interaction item.

In addition, when a writing and modification item 2410 displayed on one side of the first cognitive behavior therapy record case within the cognitive behavior therapy record screen 2400 displayed on the first terminal is selected, as illustrated in FIG. 25, the first terminal displays a cognitive behavior therapy record input screen 2500 corresponding to the selected writing and modification item 2410 provided from the server 200.

In addition, the first terminal receives a plurality of first input values corresponding to the plurality of input items included in the cognitive behavior therapy record input screen 2500 displayed on the first terminal according to the user input. Here, the first plurality of input values include the “Please display any area that has improved slightly through training” content (e.g., including “It's uncomfortable, but it's getting better,” “Tried something new,” “You can control your anxiety to some extent,” etc.), the “What actually happened in relation to an area I was worried about before training?” content (e.g., including “Other people do not think it's strange when they see my way of speaking,” etc.), the “Please re-evaluate your thoughts before training” content (e.g., including degree of confidence in “I think people will think I'm strange when they see me: Maybe so,” etc.), the “how I feel now” content (e.g., including “happiness: 70%—There's no problem at all now. I'm very happy. Annoyed—30%—I'm happy, but I guess I can't help but feel a little annoyed,” etc.), the “What did you learn?” content (e.g., including “I learned a lot. I still have a lot to do, but I plan to keep doing it,” etc.) etc.

In addition, the first terminal is linked with the server 200 to apply the first plurality of received input values to the first cognitive behavior therapy record information related to the first cognitive behavior therapy record case, and as illustrated in FIG. 26, displays the cognitive behavior therapy record screen 2600 in the state in which the first plurality of input values is applied.

As another example, when a new cognitive behavior therapy record sheet writing item 2420 is selected within the cognitive behavior therapy record screen 2400 displayed on the first terminal, as illustrated in FIG. 27, the first terminal displays a new cognitive behavior therapy record sheet writing screen 2700 corresponding to the selected new cognitive behavior therapy record sheet writing item 2420 provided from the server 200.

In addition, the first terminal receives a plurality of second input values corresponding to the plurality of other input items included in the new cognitive behavior therapy record sheet writing screen 2700 displayed on the first terminal according to the user input. Here, the plurality of second input values include the cognitive behavior therapy record sheet title (e.g. secondary cognitive behavior therapy record), the “What are situations that make you anxious?” content (e.g., including “I feel anxious in bathrooms and around other people,” etc.) as the input value before training, the “How are you feeling at this time?” content (e.g., including “anger/rage: 80%—I get angry when other people are around when I go to the bathroom,” “anxiety: 50%—I feel anxious when there are people in the bathroom,” etc.), the “What are your thoughts in that situation?” content (e.g., including “I think people will think I'm strange when they see me, degree of confidence: Maybe so, cognitive error: obsession,” etc.), the “Please correct my wrong thoughts” content (e.g., including “No one saw me,” “I think it was my mistake,” etc.), the behavioral goal setting content (e.g., including “I won't be hasty in judging from now on,” etc.) In addition, the first terminal is linked with the server 200 to generate the second plurality of received input values as a new secondary cognitive behavior therapy record case, applies the second cognitive behavior therapy record information related to the created second cognitive behavior therapy record case, and as illustrated in FIG. 28, displays the cognitive behavior therapy record screen 2800 in the state in which the second plurality of input values are applied (S310).

In addition, when a chat room related to any one topic talk is selected from one or more chat rooms related to the topic talk displayed in the preset chat room display area among the app execution result screen displayed on the corresponding terminal 100, the terminal 100 is linked with the server 200 to enter (or participate in) the chat room related to the selected topic talk. In this case, the terminal 100 displays the waiting screen according to the entry into the chat room. Here, the waiting screen includes the type of the corresponding chat room, the topic, the chat room name, the keyword (or hashtag), the nickname (or chat name) of the user who enters the corresponding chat room, the chat temperature, and the number of chats (or the number of times the user has participated in the chat room), etc. In this case, the chat temperature may be calculated based on the number of chats, the received evaluation results, etc., and the concept of level or points may be applied instead of the chat temperature. That is, for the level or point, the level or the preset number of points may be calculated according to a plurality of preset grades based on the number of chats, the received evaluation results, etc.

In addition, when the room leader who opens the corresponding chat room selects the preset start menu while the number of people (or number of participants) set in the chat room related to the topic talk is met, the terminal 100 selects one or more keywords the user wants to say, one or more keywords the user wants to hear, one or more additional keywords, etc., according to the user selection of the terminal 100 from the screen for selecting the keyword that the user of the terminal 100 wants to say or hear.

In addition, the terminal 100 is linked with the server 200 and one or more other terminals (not illustrated) owned by one or more other users in the chat room related to the topic talk, to perform the chat function related to the specific topic. In this case, the terminal 100 is linked with the server 200 and uses the avatar (or character) preset in the terminal 100 to change (or apply/reflect) and display the expression (e.g., including happiness, joy, excitement, passion, pleasure, frustration, outrage, sadness, surprise, pain, rage, etc.) of the corresponding avatar according to the analysis (e.g., including a change in a user's gaze, a change in facial expression, a voice tremor, a change in heart rate, etc.) of the psychological state of the corresponding user's voice and/or facial expression while the user of the terminal 100 is chatting.

That is, the terminal 100 sequentially targets a plurality of users (e.g., including the user of the terminal 100, other users of terminals, etc.) in the chat room related to the corresponding topic talk, and performs the chat function related to the topic and keyword of the corresponding chat room once sequentially (or under the control of the room leader).

In addition, the terminal 100 performs free talking (or a free talking chat function) with a plurality of users participating in the corresponding chat room for a preset time (e.g., including 2 minutes) according to the keywords (e.g., interpersonal relationships, junior management, company life, boss, career, office worker, company, stress, other/adding keyword, etc.) set by the room leader who creates the chat room related to the topic talk.

In this way, the terminal 100 may freely perform the chat function with other users in relation to the topic, the keyword, etc., in which the user of the terminal 100 is interested.

As an example, when a first chat room (e.g., “I'm worried about my junior who is better at work than me”) 730 is selected from one or more chat rooms related to the topic talk displayed in the preset chat room display area among the “free expression” app execution result screen 700, as illustrated in FIG. 29, the first terminal is linked with the server 200 to display the first chat room screen (or first chat room waiting screen) 2900 corresponding to the selected second chat room 730.

In addition, when the Seongsu-dong native, the room leader who opens the corresponding chat room (or terminal owned by the Seongsu-dong native), selects the preset start menu while the preset number of people (e.g., 4 people) in relation to the first chat room is met, as illustrated in FIG. 30, the first terminal displays the keyword selection screen 3000.

In addition, after one or more keywords the user wants to talk about (e.g., including company life, etc.), one or more keywords the user wants to hear (e.g., including company life, junior management, etc.), etc., depending on the selection of the first user of terminal are selected from the keyword selection screen 3000 displayed on the first terminal, when the completion menu displayed on one side of the screen is selected, the first terminal maintains an additional standby state until the remaining three users each complete their keyword selection.

In addition, when all four users participating in the first chat room complete the keyword selection, as illustrated in FIGS. 31 to 36, the first terminal is linked with the second terminal 100 and the fourth terminal 100 participating in the first chat room and the server 200 to perform the chat function for a preset time in order to target the plurality of keywords selected by the four users (S360).

Thereafter, when the topic talk (or topic talk chat function) related to the chat room ends, the terminal 100 is linked with the server 200 to perform a self-evaluation function for the user of the corresponding terminal 100 and a counterpart evaluation function for one or more other users participating in the corresponding chat room. Here, examples of the self-evaluation function include an evaluation function for an overall satisfaction item (e.g., including disappointed, average, satisfied, other, etc.) with one's chat, an anxiety item, a confidence/assertiveness item, a mind control item, a communication skills/logic/persuasive power item, an immersion item, an empathy/listening skills item, etc. In this case, the anxiety item, the confidence/assertiveness item, the mind control item, the communication skills/logic/persuasive power item, the immersion item, the empathy/listening skills item, etc., may be selected in a preset range (e.g., 0 to 100 points) depending on the user's selection. In addition, the counterpart evaluation function includes an evaluation function for a satisfaction item (e.g., including disappointed, average, satisfied, etc.) of overall chats by different users, other user-specific chat evaluation items (e.g., including “He doesn't seem anxious at all, he's confident and assertive,” “He's good at controlling his emotions,” “He's good at communicating logically,” “He's good at immersing himself in the situation,” “He listens well and has good empathy,” etc.), etc.

That is, when the topic talk (or topic talk chat function) related to the chat room ends, the terminal 100 transmits the information related to the self-evaluation according to the user input of the terminal 100, receives the information, etc., related to counterpart evaluation of other users, and transmits the information related to the received self-evaluation, the information related to the received counterpart evaluation of other users, the identification information of the terminal 100, etc., to the server 200.

In addition, the server 200 receives (or collects) the information related to the self-evaluation from the plurality of terminals 100 participating in the chat room related to the corresponding topic talk, the information related to the counterpart evaluations by other users, the identification information of the terminal 100, etc.

For example, when the topic talk in the first chat room ends, as illustrated in FIG. 37, for the self-evaluation function, the first terminal displays a screen 3700 for selecting the information on whether the user's chat was satisfactory.

In addition, when any one item (e.g., “I am satisfied”) is selected from the displayed screen 3700, as illustrated in FIG. 38, for the self-evaluation function, the first terminal displays an additional screen 3800.

In addition, the first terminal receives a first input value (e.g., anxiety: 65 points, confidence/assertiveness: 40 points, mind control: 70 points, communication skills/logic/persuasive power: 85 points, immersion: 40 points, empathy/listening skills: 30 points, etc.) according to the first user of terminal selection for the anxiety item, the confidence/assertiveness item, the mind control item, the communication skills/logic/persuasive power item, the immersion item, the empathy/listening skills item, etc., included in the displayed additional screen 3800.

In addition, as illustrated in FIG. 39, the first terminal displays a screen 3900 for selecting information on whether the chat of a specific user (e.g., including Seongsu-dong native, Bangbae-dong native, Samseong-dong native, etc.) was satisfactory for the counterpart evaluation function for other users participating in the first chat room.

In addition, when any one item is selected from the displayed screen 3900, as illustrated in FIG. 40, for the counterpart evaluation function, the first terminal displays an additional screen 4000.

In addition, the first terminal receives an input value according to the selection of the first user of terminal for the chat evaluation item included in the displayed other additional screen 4000.

In this way, the first terminal receives a second input value that includes a first counterpart evaluation input value (e.g., including average, good listening, good empathy, etc.) related to the Seongsu-dong native who participates in the first chat room, a second counterpart evaluation input value (e.g., including satisfied, confident and assertive, etc.) related to the Bangbae-dong native, a third counterpart evaluation input value (e.g., including average, good at controlling emotions, etc.), related to the Samseong-dong native, etc.

In addition, the first terminal transmits an item (e.g., I am satisfied) selected in relation to the self-evaluation, the received first input value (e.g., including anxiety 65 points, confidence/assertiveness 40 points, communication skills/logic/persuasive power 85 points, immersion 40 points, empathy/listening skills 30 points, etc.), a second input received in relation to the counterpart evaluation, the identification information of the first terminal, etc., to the server 200.

In addition, the server 200 receives an item (e.g., I am satisfied) related to the self-evaluation transmitted from the first terminal participating in the first chat room, the first input value (e.g., including anxiety 65 points, confidence/assertiveness 40 points, mind control 70 points, communication skills/logic/persuasive power 85 points, immersion 40 points, empathy/listening skills 30 points, etc.), a second input value related to the counterpart evaluation, the identification information of the first terminal, etc. (S330).

In addition, the server 200 generates chat results for each user participating in the corresponding chat room according to the performance of the self-evaluation function and the counterpart evaluation function performed by each of the plurality of terminals participating in the topic talk related to the corresponding chat room. Here, the chat result (or chat result information) includes a total average score item according to self-evaluation, an overall rating display item, an item for displaying scores (e.g., including an anxiety score, a confidence/assertiveness score, a mind control score, a communication skills/logic/persuasive power score, an immersion score, an empathy/listening skills score, etc.) for each self-evaluation item and an average score display item related to the topic talk, an evaluation item received according to the counterpart's evaluation, the number saves display item for displaying the number of times the chat content from the corresponding chat room can be saved (or downloaded), a chat recording storing item for storing the chat content, a chat time display item for displaying the total chat time in the corresponding chat room and the time the corresponding user spoke, a chat content output item for re-listening to the chat content in the corresponding chat room, etc. In addition, examples of the received evaluation item include the number of votes item received for each chat satisfaction item, the number of votes item received for each chat evaluation item, etc.

That is, the server 200 generates different chat results for each user participating in the corresponding chat room based on the information related to the self-evaluation received (or collected) from each of the plurality of terminals 100 participating in the chat room related to the corresponding topic talk, the information related to the counterpart evaluation of other users, etc.

In addition, the server 200 transmits the generated chat results for each user to the terminal 100 owned by each user.

In addition, the server 200 updates (or calculates/computes) the free expression index, the anxiety index, the chat temperature (or level/point), etc., related to the user based on another chat results generated in relation to the user of the terminal 100.

As an example, the server 200 generates the first chat result to the fourth chat result for each user based on the item selected in relation to the self-evaluation, the first input value, the second input value related to the counterpart evaluation, etc., in relation to four users participating in the first chat room.

In addition, the server 200 transmits the generated first chat result related to the first user, a Yeoksam-dong native, to the first terminal, transmits a generated second chat result related to the second user, a Seongsu-dong native, to the second terminal related to the second user, transmits the generated third chat result related to the third user, a Bangbae-dong native, to the third terminal related to the third user, and transmits the fourth generated chat result related to the fourth user, a Samseong-dong native, to the fourth terminal related to the fourth user.

In addition, the server 200 applies the first chat result generated in relation to the first user of the first terminal in the first “free expression” index, the first anxiety index, the first chat temperature, etc., related to the first user to update the first “free expression” index, the first anxiety index, the first chat temperature, etc., related to the first user.

In addition, the server 200 updates the second “free expression” index, the second anxiety index, the second chat temperature, etc., related to the second user based on the second chat result generated in relation to the second user (e.g., the Seongsu-dong native) of the second terminal, updates the third “free expression” index, the third anxiety index, the third chat temperature, etc., related to the third user based on the third chat result generated in relation to the third user (e.g., the Bangbae-dong native) of the third terminal, and updates the fourth “free expression” index, the fourth anxiety index, the fourth chat temperature, etc., related to the fourth user based on the fourth chat result generated in relation to the fourth user (e.g., the Samseong-dong native) of the fourth terminal (S340).

Thereafter, the terminal 100 receives the chat results related to the user of the terminal 100 according to the performance of the self-evaluation function and the counterpart evaluation function transmitted from the server 200.

In addition, the terminal 100 displays (or outputs) the received chat results.

As an example, the first terminal receives the first chat result transmitted from the server 200.

In addition, as illustrated in FIG. 41, the first terminal displays the received first chat result 4100 (S350).

In addition, when a chat room related to any one role-playing is selected from one or more chat rooms related to the role-playing for the specific role (or situation) displayed in the preset chat room display area among the app execution result screen displayed on the corresponding terminal 100, the terminal 100 is linked with the server 200 to enter (or participate in) the chat room related to the selected role-playing. In this case, the terminal 100 displays the waiting screen according to the entry into the chat room. Here, the waiting screen includes the type of the corresponding chat room, the topic, the chat room name, the keyword (or hashtag), the nickname (or chat name) of the user in the corresponding chat room, the chat temperature, and the number of chats (or the number of times the user has participated in the chat room), etc. In this case, the chat temperature may be calculated based on the number of chats, the received evaluation results, etc., and the concept of level or points may be applied instead of the chat temperature. That is, for the level or points, the level or the preset number of points may be calculated according to a plurality of preset grades based on the number of chats, the received evaluation results, etc.

In addition, when the room leader who opened the corresponding chat room selects the preset start menu while the number of people (or the number of participants) set in the chat room related to the corresponding role-playing is met, the terminal 100 displays the my role description screen for confirming the description of the role of the user of the terminal 100 in the chat room related to the corresponding role-playing and the emotions felt by the user.

In addition, the terminal 100 is linked with one or more other terminals (not shown) owned by one or more other users in the chat room related to the role-playing and the server 200 to perform the chat function according to the role-playing while alternately performing the main and sub roles with the user and other users. In this case, when the chat view menu displayed on one side of the screen of the terminal 100 is selected to confirm the appropriate chat for the role while performing the chat function according to the performance of the main role or sub-role, the terminal 100 may display information (or lines/tips) on the related main role or sub-role on one side of the screen. In addition, the terminal 100 is linked with the server 200 and uses the avatar (or character) preset in the terminal 100 to change (or apply/reflect) and display the expression (e.g., including happiness, joy, excitement, passion, pleasure, frustration, outrage, sadness, surprise, pain, rage, etc.) of the corresponding avatar according to the analysis (e.g., including a change in a user's gaze, a change in facial expression, a voice tremor, a change in heart rate, etc.) of the psychological state of the corresponding user's voice and/or facial expression while the user of the terminal 100 is chatting.

That is, the terminal 100 sequentially targets a plurality of users (e.g., including the user of the terminal 100, other users of terminals, etc.) in the chat room related to the role-playing, and performs the chat function according to the main role and sub role of the corresponding chat room once sequentially (or under the control of the room leader). Here, the main role should directly answer (or deal with/respond to) to a given situation spontaneously without any set chat, and the sub-role is responsible for describing the corresponding situation description, confirming the answer of the user performing the main role, etc. In this case, the user performing the main role should answer questions on the “my role” description screen (e.g., including questions such as “How do you feel in this situation?” etc.) for confirming the description of the role and the emotion felt by the user, and the answer to this question may be used to identify the automatic thoughts in the cognitive reconstruction therapy, one of the cognitive behavior therapy programs, to evaluate the automatic thought. Here, the automatic thoughts represent (or mean) thoughts, ideas, etc., for interpreting the situation when suddenly faced with a certain situation.

In this way, the terminal 100 may freely perform the chat function with other users according to the role-playing in relation to the topic, the keyword, etc., in which the user of the terminal 100 is interested.

As an example, when the second chat room (e.g., a junior who should refuse a boss who makes unreasonable demands at work) 740 is selected from one or more chat rooms related to role-playing for a specific role (or situation) displayed in the preset chat room display area among the “free expression” app execution result screen 700, as illustrated in FIG. 42, the first terminal is linked with the server 200 to display a second chat room screen (or second chat room waiting screen) 4200 corresponding to the selected second chat room 740.

In addition, when the Seocho-dong native, the room leader who opens the corresponding chat room (or on a terminal owned by the corresponding Seocho-dong native), selects the preset start menu while the preset number of people (e.g., 3 people) (or the terminal owned by the Seocho-dong native) in relation to the second chat room is met, as illustrated in FIG. 43, the first terminal displays a “my role” description screen 4300 for confirming the description of the role of the first user of the first terminal in the second chat room related to the corresponding role-playing and the emotions felt by the user.

In addition, after a score (e.g., 25 points) of a “What emotions do you feel in this situation?” item is selected depending on the selection of the first user of terminal on the “my role” description screen 4300 displayed on the first terminal, when the confirmation menu displayed on one side of the screen is selected, the first terminal maintains an additional waiting state until the remaining two users each complete the confirmation of the content.

In addition, when all three users participating in the second chat room complete confirmation of the content, as illustrated in FIGS. 44 to 47, the first terminal is linked with the twelfth terminal 100 to the thirteenth terminal 100 participating in the second chat room and the server 200 to perform the chat functions for a preset time in order while changing the roles in relation to the chat content in the corresponding role-playing (S360).

Thereafter, when the role-playing (or role-playing chat function) related to the chat room ends, the terminal 100 is linked with the server 200 to perform a self-evaluation function for the user of the corresponding terminal 100 and a counterpart evaluation function for one or more other users participating in the corresponding chat room. Here, the self-evaluation function includes an evaluation function for overall satisfaction item (e.g., including disappointed, average, satisfied, other, etc.) with the user's chat, an anxiety item, a confidence/assertiveness item, a mind control item, a communication skills/logic/persuasive power item, an immersion item, an empathy/listening skills item, etc. In this case, the anxiety item, the confidence/assertiveness item, the mind control item, the communication skills/logic/persuasive power item, the immersion item, the empathy/listening skills item, etc., may be selected in a preset range (e.g., 0 to 100 points) depending on the user's selection. In addition, the counterpart evaluation function includes an evaluation function for a satisfaction item (e.g., including disappointed, average, satisfied, etc.) of overall chats by different users, other user-specific chat evaluation item (e.g., including “He doesn't seem anxious at all, he's confident and assertive, “He's good at controlling his emotions,” “He's good at communicating logically,” “He's good at immersing himself in the situation,” “He listens well and has good empathy,” etc.), etc.

That is, when the role-playing (or role-playing chat function) related to the chat room ends, the terminal 100 transmits the information related to the self-evaluation according to the user input of the terminal 100, receives the information, etc., related to counterpart evaluation of other users, and transmits the information related to the received self-evaluation, the information related to the received counterpart evaluation of other users, the identification information of the terminal 100, etc., to the server 200.

In addition, the server 200 receives (or collects) the information related to the self-evaluation from the plurality of terminals 100 participating in the chat room related to the corresponding role-playing, the information related to the counterparty evaluations by other users, the identification information of the terminal 100, etc.

For example, when the role-playing in the second chat room ends, as illustrated in FIG. 48, for the self-evaluation function, the first terminal displays a screen 4800 for selecting information on whether the user's chat was satisfactory.

In addition, when any one item (e.g., “I am satisfied”) is selected from the displayed screen 4800, as illustrated in FIG. 49, for the self-evaluation function, the first terminal displays an additional screen 4900.

In addition, the first terminal receives a third input value (e.g., anxiety: 30 points, confidence/assertiveness: 50 points, mind control: 70 points, communication skills/logic/persuasive power: 80 points, immersion: 50 points, empathy/listening skills: 40 points, etc.) according to the first user of terminal selection for the anxiety item, the confidence/assertiveness item, the mind control item, the communication skills/logic/persuasive power item, the immersion item, the empathy/listening skills item, etc., included in the displayed additional screen 4900.

In addition, as illustrated in FIG. 50, the first terminal displays a screen 5000 for selecting information on whether the chats of other specific users (e.g., including the Seocho-dong native, the Nonhyeon-dong native, etc.) were satisfactory for the counterpart evaluation function for other users participating in the corresponding second chat room.

In addition, when any one item is selected from the displayed screen 5000, as illustrated in FIG. 51, for the counterpart evaluation function, the first terminal displays another additional screen 5100.

In addition, the first terminal receives an input value according to the selection of the first user of terminal for the chat evaluation item included in the displayed other additional screen 5100.

In this way, the first terminal receives a fourth input value that includes an eleventh counterpart evaluation input value (e.g., including average, good at controlling emotions, etc.) related to the Seocho-dong native who participates in the second chat room, a twelfth counterpart evaluation input value (e.g., including satisfied, “He's good at communicating logically,” etc.) related to the Nonhyeon-dong native, etc.

In addition, the first terminal transmits an item (e.g., “I am satisfied”) selected in relation to the self-evaluation, the received third input value (e.g., including anxiety 30 points, confidence/assertiveness 50 points, mind control 70 points, communication skills/logic/persuasive power 80 points, immersion 50 points, empathy/listening skills 40 points, etc.), a fourth input received in relation to the counterpart evaluation, the identification information of the first terminal, etc., to the server 200.

In addition, the server 200 receives an item (e.g., “I am satisfied”) selected in relation to the self-evaluation transmitted from the first terminal participating in the corresponding second chat room, the third input value (e.g., including anxiety 30 points, confidence/assertiveness 50 points, mind control 70 points, communication skills/logic/persuasive power 80 points, immersion 50 points, empathy/listening skills 40 points, etc.), a fourth input value related to the counterpart evaluation, the identification information of the first terminal, etc. (S370).

Thereafter, the server 200 generates other chat results for each user participating in the corresponding chat room according to the performance of the self-evaluation function and the counterpart evaluation function performed by each of the plurality of other terminals participating the role-playing related to the corresponding chat room. Here, the other chat results (or other chat result information) include a situation description and scoring item for displaying one's initial feelings about the situation, a “my first emotion of how I felt about the situation” display item, a total average score item according to self-evaluation, an overall rating display item, an item for displaying scores (e.g., including an anxiety score, a confidence/assertiveness score, a mind control score, a communication skills/logic/persuasive power score, an immersion i score, an empathy/listening skills score, etc.) for each self-evaluation item and an average score related to the corresponding role-playing, an evaluation item received according to the counterpart's evaluation, the number saves display item for displaying the number of times content from the corresponding chat room can be saved (or downloaded), a chat recording storing item for storing the corresponding chat content, a chat time display item for displaying a total chat time in the corresponding chat room and the time the user spoke, a chat content output item for re-listening to the chat content from the corresponding chat room, etc. In addition, the received evaluation item includes a number of votes item for each chat satisfaction item, a number of votes item for each chat evaluation item, etc.

That is, the server 200 generates different chat results for each user participating in the corresponding chat room based on the information related to the self-evaluation received (or collected) from each of the plurality of terminals 100 participating in the chat room related to the corresponding role-playing, the information related to the counterpart evaluation of other users, etc.

In addition, the server 200 transmits the generated other chat results for each user to the terminal 100 owned by each user.

In addition, the server 200 updates (or calculates/computes) the free expression index, the anxiety index, the chat temperature (or level/point), etc., related to the user based on other chat results generated in relation to the user of the terminal 100.

As an example, the server 200 generates the eleventh chat result to the thirteenth chat result for each user based on the item selected in relation to the self-evaluation, the third input value, the fourth input value related to the counterpart evaluation, etc., in relation to three users participating in the second chat room.

In addition, the server 200 transmits the generated eleventh chat result related to the first user, the Yeoksam-dong native, to the first terminal, transmits a generated twelfth chat result related to the twelfth user, the Seocho-dong native, to a twelfth terminal related to the twelfth user, and transmits a generated thirteenth chat result related to the thirteenth user, the Nonhyeon-dong native, to a thirteenth terminal related to the 13th user.

In addition, the server 200 applies the eleventh chat result generated in relation to the first user of the first terminal in the first “free expression” index, the first anxiety index, the first chat temperature, etc., related to the first user to update the first “free expression” index, the first anxiety index, the first chat temperature, etc., related to the first user.

In addition, the server 200 updates the twelfth “free expression” index, the twelfth anxiety index, the twelfth chat temperature, etc., related to the twelfth user based on the twelfth chat result generated in relation to the twelfth user of the twelfth terminal (e.g., the Seocho-dong native), and updates the thirteenth “free expression” index, the thirteenth anxiety index, the thirteenth chat temperature, etc., related to the thirteenth user based on the thirteenth chat result generated in relation to the thirteenth user of the thirteenth terminal (e.g., the Nonhyeon-dong native) (S380).

Thereafter, the terminal 100 receives other chat results related to the user of the terminal 100 according to the performance of the self-evaluation function and the counterpart evaluation function transmitted from the server 200.

In addition, the terminal 100 displays (or outputs) the other received chat results.

As an example, the first terminal receives the eleventh chat result transmitted from the server 200.

In addition, as illustrated in FIG. 52, the first terminal displays the received eleventh chat result 5200 (S390).

In addition, when a preset room creation menu is selected from the app execution result screen displayed on the terminal 100, to create a new chat room in relation to the category the user wants, the terminal 100 displays the room creation screen corresponding to the selected room creation menu. Here, the room creation screen includes a room type selection menu for selecting a room type, etc. In addition, the room type selection menu includes the topic talk item for free chat between users with similar interests, a role-playing item for practicing expressing emotions and opinions by setting up situations, a theme practice item for developing (or practicing) adaptability and appropriate responsiveness by creating the theme that may cause tension, etc. In this case, the room creation menu may be activated and displayed only on the terminal 100 of a pre-registered (or set) expert terminal 300 or a paid member (or subscription member) for whom chat room creation rights are set.

As an example, when a preset room creation menu 750 is selected from the “free expression” app execution result screen 700, as illustrated in FIG. 53, the first terminal displays a room creation screen 5300 corresponding to the selected room creation menu (750).

Thereafter, when the preset theme practice item is selected from the room creation screen displayed on the corresponding terminal 100, to develop (or practice) adaptability and appropriate responsiveness by creating the theme that may cause tension, the terminal 100 displays a spectator type setting screen corresponding to the selected theme practice item. Here, the spectator type setting screen includes a no spectator item, a spectator item, etc., for selecting the theme type according to the presence or absence of a spectator.

For example, when a theme practice item 5310 is selected from the room creation screen 5300 displayed on the first terminal, as illustrated in FIG. 54, the first terminal displays a spectator type setting screen 5400 corresponding to the selected theme practice item 5310 (S410).

Thereafter, when a preset no spectator item is selected from the spectator type setting screen displayed on the terminal 100, to create a theme practice chat room for performing the chat function with the question-and-answer, the terminal 100 displays a theme setting screen corresponding to the selected no spectator item. Here, the theme setting screen (or question theme setting screen) includes a question-and-answer item, a next item, etc. In this case, the chat room without a spectator (or theme practice chat room without a spectator) may be configured to be created in the pre-registered (or set) expert terminal 300 or the terminal 100 of a paid member (or subscription member) where the chat room creation permission is set.

As an example, when a no spectator item 5410 is selected from the spectator type setting screen 5400 displayed on the first terminal, as illustrated in FIG. 55, the first terminal displays a theme setting screen 5500 corresponding to the selected no spectator item 5410.

Thereafter, when the preset question-and-answer item is selected from the theme setting screen displayed on the terminal 100, the terminal 100 displays the questionnaire setting screen for receiving the self-introduction or question content. Here, the questionnaire setting screen may be configured in different forms (or formats) from the perspective of a general user or an expert/trainer, and includes a simple self-introduction/question purpose section, one or more questionnaire items the user would like to ask other participants, a questionnaire addition item, a registration item, etc. Here, the one or more questionnaire items may include at least one basic question (e.g., “Briefly introduce yourself,” “Have you ever participated in outside activities or clubs?” “What kind of work you would like to do at the company?” etc.) that is preset by default. Here, the registration item remains deactivated by default, and may be displayed by switching from the deactivated state to the activated state when the input values for the simple self-introduction/question purpose item, the questionnaire item, etc., are input (or received).

In addition, the terminal 100 receives questionnaire setting information including the plurality of input values corresponding to the plurality of input items included in the questionnaire setting screen displayed on the terminal 100 according to the user input. Here, the questionnaire setting information includes a brief self-introduction/purpose of the question, content of one or more questionnaires to be asked to other participants, etc.

As an example, when a question-and-answer item 5510 is selected from the theme setting screen 5500 displayed on the first terminal, as illustrated in FIG. 56, the first terminal displays a questionnaire setting screen 5600 corresponding to the selected question-and-answer item 5510.

In addition, the first terminal includes a fifth input value that includes a fifth simple self-introduction/question purpose (e.g., “I am a 27-year-old job seeker, and I have a face-to-face interview at OO company next week. If you are working at an OO company, I need help from a speech expert!”) corresponding to the plurality of input items included in the questionnaire setting screen 5600 displayed on the first terminal, a fifth one or more questions/answer sheets (e.g., “Briefly introduce yourself,” “Have you ever participated in outside activities or club activities?” “What kind of work do you want to do at the company?” etc.), etc (S430).

Thereafter, after receiving the questionnaire setting information from the questionnaire setting screen, when the registration item displayed on one side of the questionnaire setting screen is selected, the terminal 100 displays the detailed theme setting screen to which the received questionnaire setting information is applied (or reflected). Here, the detailed theme setting screens (or other theme practice chat room creation screen/question-and-answer theme setting screens) include a category item (e.g., including blind date practice without a spectator, etc.), a brief self-introduction/question purpose item, one or more questionnaire content items, a room title input item for selecting the room title, a “number of general users who can participate in training” input item (e.g., no, 1, 2, 3, 4 people, etc., male/female) for selecting the number of general users who can participate in training, a “number of experts who can participate in feedback” input item (for example, include 1, 2, 3, etc.) for selecting the number of experts who can participate in feedback, a room creation item, etc. Here, the room creation item remains deactivated by default, and may be displayed by switching from the deactivated state to the activated state when the input values for the category item, the brief self-introduction/question purpose item, the one or more questionnaire content items, the room title input item, the “number of general users who can participate in training” input item, the “number of experts that can participate in feedback” input item, etc., are input (or received).

For example, after receiving the fifth input value on the questionnaire setting screen 5600, when a registration item 5610 displayed on one side of the questionnaire setting screen 5600 is selected, as illustrated in FIG. 57, the first terminal displays a detailed theme setting screen 5700 to which the fifth input value is applied (S440).

Thereafter, the terminal 100 receives other detailed theme setting information including the plurality of other input values corresponding to the plurality of other input items included in the detailed theme setting screen displayed on the terminal 100 according to the user input. Here, the detailed theme setting information includes the category (e.g., question-and-answer without a spectator), brief self-introduction/question purpose, content of one or more questions the user wants to ask other participants, a room title, and the number of general users who can participate in training, the number of experts who can participate in feedback, etc.

In addition, after receiving the detailed theme setting information on the detailed theme setting screen displayed on the terminal 100, when the room creation item (or creation item) displayed on one side of the detailed theme setting screen is selected, the terminal 100 is linked with the server 200 to create (or open) a new theme practice chat room (or question-and-answer theme practice chat room) corresponding to the received detailed theme setting information. In this case, the theme practice chat room creation may be performed in the terminal 100 that is performing a subscription function in advance or in the terminal 100 that has paid a preset usage amount for each topic to create the theme practice chat room. In addition, the terminal 100 displays the waiting screen according to the entry into the chat room. Here, the waiting screen includes the type of the corresponding chat room, the topic, the chat room name, the keyword, the nickname (or chat name) of the user in the corresponding chat room, the chat temperature, and the number of chats (or the number of times the user has participated in the chat room), etc. In this case, the chat temperature may be calculated based on the number of chats, the received evaluation results, etc., and the concept of level or points may be applied instead of the chat temperature. That is, for the level or points, the level or the preset number of points may be calculated according to a plurality of preset grades based on the number of chats, the received evaluation results, etc.

As an example, the first terminal receives a sixth input value that includes a sixth category (e.g., question-and-answer without a spectator) corresponding to a plurality of other input items included in the detailed theme setting screen 5700 displayed on the first terminal, a sixth brief self-introduction/question purpose (e.g., “I am a 27-year-old job seeker and have a face-to-face interview at an OO company next week. If you are working at an OO company, I need help from a speech expert!”), a sixth one or more questions/answer sheets (e.g., including “Briefly introduce yourself,” “Have you ever participated in outside activities or club activities?” “What kind of work do you want to do at the company?” etc.), a sixth room title (e.g., speech know-how related to documents and interviews), the sixth number (e.g., 1 person) of general users who can participate in training, the sixth number of experts (e.g., 2 people) who can participate in feedback, etc. In this case, the sixth simple self-introduction/question purpose and the sixth one or more questions/answer sheets may be automatically applied (or input) based on the fifth input value.

In addition, after receiving the sixth input value from the detailed theme setting screen 5700, when a room creation item 5710 displayed on one side of the detailed theme setting screen 5700 is selected, the first terminal is linked with the server 200 to create the question-and-answer interview theme practice chat room corresponding to the received sixth input value, and enters the created question-and-answer interview theme to display a corresponding question-and-answer interview theme practice chat room screen (or question-and-answer interview theme practice chat room waiting screen) 5800 as illustrated in FIG. 58 (S450).

In addition, when a preset spectator item is selected from the spectator type setting screen displayed on the terminal 100, to create a theme practice chat room in the form of a free theme or discussion theme, the terminal 100 displays other theme setting screens corresponding to the selected spectator item. Here, the other theme setting screen (or free/discussion theme setting screen) includes a free theme item (e.g., including blind date practice item, phone practice item, interview practice item, business report practice item, small-scale presentation practice item, large-scale presentation practice item, singing practice item, etc.), a discussion theme item (e.g., including professional discussion item, pros and cons discussion item, etc.), a registration item, etc.

The embodiment of the present invention mainly describes performing the creation of the chat room with a spectator (or theme practice chat room with a spectator) in the terminal 100, but is not limited thereto, and the chat room with a spectator may also be configured to be created only in the pre-registered (or set) expert terminal 300 where the chat room creation right is set.

As an example, when a spectator item 5420 is selected from the spectator type setting screen 5400 displayed on the first terminal, as illustrated in FIG. 59, the first terminal displays other theme setting screen 5900 corresponding to the selected spectator item 5420 (S460).

Thereafter, when any one theme item is selected from other theme setting screens displayed on the terminal 100, the terminal 100 displays other detailed theme setting screens for receiving the room title, the number of participants in training, or the maximum number of spectators. Here, the other detailed theme setting screens (or other theme practice chat room creation screens/free/discussion theme setting screen) include a category item (e.g., including blind date practice with a spectator, phone practice with a spectator, etc.), a room title input item for selecting the room title, a “number of general users who can participate in training” input item (e.g., 1, 2, 3, 4 people, etc., male/female) for selecting the number of general users who can participate in training, a “maximum number of spectators” input item (e.g., 5, 10, 20 people, etc.) for selecting the number of users who can be spectate, a room creation item, etc. In this case, when a professional discussion item or pros and cons discussion item included in the discussion theme item in the other theme setting screens is selected, before displaying the other detailed theme setting screens, the terminal 100 may first display a discussion topic setting screen, receive one or more discussion topics (or discussion topic information/content) according to the user input, and then display the other detailed theme setting screens to which one or more discussion topics are applied. Here, when the room creation item is kept in a deactivated state by default, and input values for the category item, the room title input item, the “number of general users who can participate in training” input item, and the “maximum number of spectators” input item, etc., are input (or received), the room creation item may be displayed by switching from a deactivated state to an activated state.

As an example, when a blind date practice item 5910 is selected from the other theme setting screen 5900, as illustrated in FIG. 60, the first terminal displays a blind date detailed theme setting screen 6000 corresponding to the selected blind date practice item 5910.

As another example, when a professional discussion item 5920 is selected from the other theme setting screen 5900, as illustrated in FIGS. 61 and 62, the first terminal displays a discussion topic setting screen 6200 to set a discussion topic, which is additional information necessary for creating a chat room related to professional discussion.

In addition, after receiving one or more discussion topics according to the user input (e.g., including hot issues these days, “What do you think about the sesame leaf debate and shrimp debate?” “Is it okay to work 4 days a week?” etc.) in the discussion topic setting screen 6200 displayed on the first terminal, when a registration item 6210 displayed on one side of the screen is selected, as illustrated in FIG. 63, the first terminal displays a detailed professional discussion theme setting screen 6300 (S470).

Thereafter, the terminal 100 receives other detailed theme setting information including a plurality of additional input values corresponding to a plurality of additional input items included in other detailed theme setting screens displayed on the terminal 100 according to the user input. Here, the other detailed theme setting information includes categories (e.g., blind date practice with spectators, phone practice, interview practice, work reporting practice, small-scale presentation practice, large-scale presentation practice, professional discussion, pros and cons discussion, etc.), a discussion topic, a room title, the number of general users who can participate in training, the maximum number of spectators, etc.

In addition, after receiving other theme detailed setting information on other theme detailed setting screens displayed on the terminal 100, when the room creation item (or creation item) displayed on one side of other theme detailed setting screens is selected, the terminal 100 is linked with the server 200 to create (or open) a new theme practice chat room (or free theme practice chat room/discussion theme practice chat room) corresponding to the received other detailed theme setting information and enter the created new theme practice chat room. In this case, the theme practice chat room creation may be performed in the terminal 100 that is performing a subscription function in advance or in the terminal 100 that has paid a preset usage amount for each topic to create the theme practice chat room. In addition, the terminal 100 displays the waiting screen according to the entry into the chat room. Here, the waiting screen includes the type of the corresponding chat room, the topic, the chat room name, the keyword, the nickname (or chat name) of the user in the corresponding chat room, the chat temperature, and the number of chats (or the number of times the user has participated in the chat room), etc. In this case, the chat temperature may be calculated based on the number of chats, the received evaluation results, etc., and the concept of level or points may be applied instead of the chat temperature. That is, for the level or points, the level or the preset number of points may be calculated according to a plurality of preset grades based on the number of chats, the received evaluation results, etc.

As an example, the first terminal receives a seventh input value that includes a seventh category (e.g., blind date practice with a spectator) corresponding to a plurality of other input items included in the detailed blind date theme setting screen 6000 displayed on the first terminal, a seventh room title (e.g., “Can I go on a blind date?”), the seventh number of general users who can participate in training (e.g., 1 male and 1 female, etc.), the seventh maximum number of spectators (e.g., 5 people), etc.

In addition, after receiving the seventh input value, when a room creation item 6010 displayed on one side of the detailed blind date theme setting screen 6000 is selected, the first terminal is linked with the server 200 to create a new blind date theme chat room corresponding to the received seventh input value, and enters the generated new blind date theme chat room to display a new blind date theme chat room screen (or blind date theme waiting screen) 6400 as illustrated in FIG. 64.

As another example, the first terminal receives an eighth input value that includes an eighth category (e.g., professional discussion with a spectator) corresponding to a plurality of other input items included in the detailed professional discussion theme setting screen 6300 displayed on the first terminal, an eighth discussion topic (e.g., including hot issues these days: “What do you think about the sesame leaf debate and shrimp debate?” “Is it okay to work 4 days a week?” etc.), an eighth room title (e.g., hot issues these days), the eighth number of general users who can participate in training (e.g., 2 people), the eighth maximum number of spectators (e.g., 5 people), etc.

In addition, after receiving the eighth input value, when a room creation item 6310 displayed on one side of the detailed professional discussion theme setting screen 6300 is selected, the first terminal is linked with the server 200 to create a new professional discussion theme chat room corresponding to the received eighth input value, and enters the generated new professional discussion theme chat room to display a new professional discussion theme chat room screen (or professional discussion theme waiting screen) (S480).

Thereafter, when the room leader who opens the corresponding chat room selects the preset start menu while the number of people (or the number of participants) set in the chat room related to the corresponding theme practice is met, the terminal 100 is linked with one or more other terminals 100 (not illustrated) owned by one or more other users in the corresponding chat room related to the corresponding theme practice and the server 200 to perform the chat function related to the specific theme (or topic) of the corresponding chat room. In this case, in the case where it is difficult to give an answer related to the theme while performing the chat function for the theme, when the tip menu displayed on one side of the screen of the terminal 100 is selected to check the tip, and the terminal 100 may display information (or a tip) on related themes on one side of the screen. In addition, the terminal 100 is linked with the server 200 and uses the avatar (or character) preset in the terminal 100 to change (or apply/reflect) and display the expression (e.g., including happiness, joy, excitement, passion, pleasure, frustration, outrage, sadness, surprise, pain, rage, etc.) of the corresponding avatar according to the analysis (e.g., including a change in a user's gaze, a change in facial expression, a voice tremor, a change in heart rate, etc.) of the psychological state of the corresponding user's voice and/or facial expression while the user of the terminal 100 is chatting.

That is, the terminal 100 sequentially targets a plurality of users (e.g., including the user of the terminal 100, other users of terminals, etc.) in the chat room related to the corresponding theme practice, and performs the chat function related to the theme of the corresponding chat room once sequentially (or under the control of the room leader). In this case, the terminal 100 may perform the chat function related to the corresponding theme without a spectator (or in a state without a spectator) on the chat room set as no spectator, or perform the chat function related to the corresponding theme with a spectator (or in a state having a spectator) on the chat room set as spectator according to the preset maximum number of spectators.

In this way, the terminal 100 may freely perform the chat function alone or with other users in relation to a specific theme in which the user of the terminal 100 appears to be interested.

As an example, when the Yeoksam-dong native (or the first terminal related to the corresponding Yeoksam-dong native), which is the room leader who opened the corresponding chat room, selects the preset start menu, while the preset number of people (e.g., 2 people) in relation to the interview theme practice chat room of FIG. 65 is met, as illustrated in FIGS. 66 to 73, the first terminal is linked with the 22^{nd} terminal 100 and the server 200 participating in the interview theme practice chat room to perform the chat function for a preset time in order for the interview situation which is the theme practice (S490).

Thereafter, when the theme practice (or theme practice chat function) related to the question-and-answer chat room ends, the terminal 100 is linked with the server 200 to perform the self-evaluation function for the user of the terminal 100. Here, the self-evaluation function includes an evaluation function for overall satisfaction item (e.g., including disappointed, average, satisfied, other, etc.) with the user's chat, an anxiety item, a confidence/assertiveness item, a mind control item, a communication skills/logic/persuasive power item, an immersion item, an empathy/listening skills item, etc. In this case, the anxiety item, the confidence/assertiveness item, the communication skills/logic/persuasive power item, the immersion item, the empathy/listening skills item, etc., may be selected in a preset range (e.g., 0 to 100 points) depending on the user's selection.

In other words, when the theme practice (or theme practice chat function) related to the question-and-answer chat room ends, the terminal 100 receives the information related to the self-evaluation, etc., according to the user input of the terminal 100, and transmits the received information related to the self-evaluation, the identification information of the terminal 100, etc., to the server 200.

In addition, the server 200 receives (or collects) the information related to the self-evaluation, the identification information of the terminal 100, etc., from the terminal 100 that participates in the question-and-answer chat room related to the corresponding theme practice and performs the chat function.

In addition, after the user of the terminal 100 performs the chat function through the theme practice with other users, when the theme practice (or theme practice chat function) related to the free theme chat room or the discussion theme chat room ends, the terminal 100 is linked with the server 200 to perform the self-evaluation function for the user of the terminal 100 and the counterpart evaluation function for one or more other users participating in the corresponding chat room. Here, the self-evaluation function includes an evaluation function for overall satisfaction item (e.g., including disappointed, average, satisfied, others, etc.) with the user's chat, an anxiety item, a confidence/assertiveness item, a mind control item, a communication skills/logic/persuasive power item, an immersion item, an empathy/listening skills item, etc. In this case, the anxiety item, the confidence/assertiveness item, the mind control item, the communication skills/logic/persuasive power item, the immersion item, the empathy/listening skills item, etc., may be selected in a preset range (e.g., 0 to 100 points) depending on the user's selection. In addition, the counterpart evaluation function performs a feedback (or feedback evaluation/feedback information) function on the counterpart that participates in the corresponding chat, or includes evaluation functions for chat evaluation items for each user (e.g., including “He doesn't seem anxious at all,” “He's confident and assertive, “He's good at controlling his emotions,” “He's good at communicating logically,” “He's good at immersing himself in the situation,” “He listens well and has good empathy,” etc.), etc. In this case, the terminal 100 may be configured to allow the user to directly input content into the counterpart evaluation function.

That is, when the theme practice (or theme practice chat function) related to the free theme chat room or the discussion theme chat room ends, the terminal 100 transmits the information related to the self-evaluation according to the user input of the terminal 100, receives the information, etc., related to counterpart evaluation of other users, and transmits the information related to the received self-evaluation, the information related to the received counterpart evaluation of other users, the identification information of the terminal 100, etc., to the server 200.

In addition, the server 200 receives (or collects) the information related to the self-evaluation from the plurality of terminals 100 participating in the chat room related to the corresponding theme practice, the information related to the counterparty evaluations by other users, the identification information of the terminal 100, etc.

For example, when the theme practice in the interview theme practice chat room ends, as illustrated in FIG. 74, for the self-evaluation function, the first terminal displays a screen 7400 for selecting the information on whether the user's chat was satisfactory.

In addition, when any one item (e.g., “I am satisfied”) is selected from the displayed screen 7400, as illustrated in FIG. 75, for the self-evaluation function, the first terminal displays an additional screen 7500.

In addition, the first terminal receives a ninth input value (e.g., anxiety: 20 points, confidence/assertiveness: 50 points, mind control: 80 points, communication skills/logic/persuasive power: 80 points, immersion: 60 points, empathy/listening skills: 50 points, etc.) according to the first user of terminal selection for the anxiety item, the confidence/assertiveness item, the mind control item, the communication skills/logic/persuasive power item, the immersion item, the empathy/listening skills item, etc., included in the displayed additional screen 7500.

In addition, as illustrated in FIG. 76, the first terminal displays a screen 7600 for selecting feedback on the chats of other specific users (e.g., including the Banpo-dong native, etc.) to provide the counterpart evaluation function for other users who participate in the interview theme practice chat room.

In addition, a tenth input value (e.g., including “He's good at immersing in the situation,” “He's good at communicating logically,” etc.), which is the counterpart evaluation input value according to the first user of terminal input, is received in the displayed screen 7600.

In addition, the first terminal transmits an item (e.g., “I am satisfied”) selected in relation to the self-evaluation, the received ninth input value (e.g., including anxiety 20 points, confidence/assertiveness 50 points, mind control 80 points, communication skills/logic/persuasive power 80 points, immersion 60 points, empathy/listening skills 50 points, etc.), a tenth input (e.g., including “He's good at immersing in the situation,” “He's good at communicating logically,” etc.) received in relation to the counterpart evaluation, the identification information of the first terminal, etc., to the server 200.

In addition, the server 200 receives an item (e.g., “I am satisfied”) selected in relation to the self-evaluation transmitted from the first terminal participating in the corresponding interview theme practice chat room, the ninth input value (e.g., including anxiety 20 points, confidence/assertiveness 50 points, mind control 80 points, communication skills/logic/persuasive power 80 points, immersion 60 points, empathy/listening skills 50 points, etc.), a tenth input (e.g., including “He's good at immersing in the situation,” “He's good at communicating logically,” etc.) related to the counterpart evaluation, the identification information of the first terminal, etc., (S500).

Thereafter, the server 200 generates the chat result for the user of the terminal 100 participating in the corresponding chat room according to the performance of the self-evaluation function in the terminal 100 participating in the theme practice related to the question-and-answer chat room. Here, the chat result (or question-and-answer chat room information) includes total average score items according to self-evaluation, an overall rating display item, an item for displaying scores (e.g., including an anxiety score, a confidence/assertiveness score, a mind control score, a communication skills/logic/persuasive power score, an immersion score, an empathy/listening skills score, etc.) for each self-evaluation item and an average score related to the corresponding theme practice, the number of saves display item for displaying the number of times chat content from the corresponding chat room can be saved (or downloaded), a chat recording storing item for storing the corresponding chat content, a chat time display item for displaying a total chat time in the corresponding chat room and the time the user spoke, a chat content output item for re-listening the chat content from the corresponding chat room, etc.

In addition, the server 200 transmits the generated chat results to the first terminal 100.

In addition, the server 200 updates (or calculates/computes) the free expression index, the anxiety index, the chat temperature (or level/point), etc., related to the user based on other chat results generated in relation to the user of the terminal 100.

In addition, when the user of the terminal 100 performs the chat function through the theme practice with other users, the server 200 generates another chat result for each user participating in the corresponding chat room depending on the performance of the self-evaluation function and the counterpart evaluation function performed by one or more terminals participating in the theme practice related to the corresponding free theme chat room or the corresponding discussion theme chat room. Here, the other chat result (or other chat result information) includes total average score items according to self-evaluation, an overall rating display item, an item for displaying scores (e.g., including an anxiety score, a confidence/assertiveness score, a mind control score, a communication skills/logic/persuasive power score, an immersion score, an empathy/listening skills score, etc.) for each self-evaluation item and an average score related to the theme practice display item, an evaluation item received according to the counterpart's evaluation, the number of saves display item for displaying the number of times chat content from the corresponding chat room can be saved (or downloaded), a chat recording storing item for storing the chat content, a chat time display item for displaying the total chat time in the corresponding chat room and the time the corresponding user spoke, a chat content output item for re-listening to the chat content in the corresponding chat room, etc. In addition, the received evaluation item includes the number of votes item for each chat satisfaction item, the number of votes item for each chat evaluation item, an evaluation content item input directly by the counterpart, etc.

In addition, the server 200 transmits the generated other chat result for each user to the terminal 100 owned by each user.

In addition, the server 200 updates (or calculates/computes) the free expression index, the anxiety index, the chat temperature (or level/point), etc., related to the user based on other chat results generated in relation to the user of the terminal 100.

As an example, the server 200 generates a 21^{st} chat result to a 22^{nd} chat result for each user in relation to two users participating in the third chat room.

In addition, the server 200 transmits the generated 21^{st} chat result related to the first user, the Yeoksam-dong native, to the first terminal, and transmits the generated 22^{nd} chat result related to the 22^{nd} user to transmit the 22^{nd} chat result to the 22^{nd} terminal related to the 22^{nd} user, the Banpo-dong native.

In addition, the server 200 applies the 21^{st} chat result generated in relation to the first user of the first terminal in the first “free expression” index, the first anxiety index, the first chat temperature, etc., related to the first user to update the first “free expression” index, the first anxiety index, the first chat temperature, etc., related to the first user.

In addition, the server 200 updates a 22^{nd} “free expression” index, a 22^{nd} anxiety index, a 22^{nd} chat temperature, etc., related to the 22^{nd} user based on the 22^{nd} chat result generated in relation to the 22^{nd} user (e.g., the Banpo-dong native) of the 22^{nd} terminal (S510).

Thereafter, when the chat function is performed in the question-and-answer chat room, the terminal 100 receives the chat result related to the user of the terminal 100 according to the performance of the self-evaluation function transmitted from the server 200.

In addition, the terminal 100 displays (or outputs) the received chat results.

In addition, when the user of the terminal 100 performs the chat function through theme practice with other users (or performs the chat function in the free theme chat room or the discussion theme chat room), the terminal 100 receives another chat result related to the user of the terminal 100 according to the performance of the self-evaluation function and the counterpart evaluation function transmitted from the server 200.

In addition, the terminal 100 displays (or outputs) another received chat result.

As an example, the first terminal receives a 21^{st} chat result transmitted from the server 200.

In addition, as illustrated in FIG. 77, the first terminal displays a received 21^{st} chat result 7700 (S520).

In addition, when the preset expert search menu is selected from the app execution result screen displayed on the terminal 100, the terminal 100 displays the expert search screen corresponding to the selected expert search menu to perform the psychological counseling, the speech lesson, or the mentor function with an expert. Here, the expert search screen includes a sorting menu for sorting functions in specialized fields related to all, psychological counseling, speech lessons, mentoring, etc., an expert count display menu for displaying the number of registered (or active) experts, an immediately available item for providing a list of specialists who are immediately available for counseling, an expert display item for providing information on an expert, a smart filter application menu for searching for an expert according to detailed conditions, desired counseling date and time information, etc. Here, examples of the expert display item include an expert name, a counseling field (or specialty field) (e.g., psychological counseling, speech lesson, mentoring, etc.), a brief introduction, a photo (or image), a fee per session, a rating (or star rating), review information, immediate counseling availability, popularity (or number of likes), etc. In addition, the smart filter application menu includes an expert type item (e.g., including all, psychological counseling, speech lesson, mentoring, etc.), an expert gender item (e.g., including all, male, female, etc.), an expert level (or badge) item (e.g. including all, general with less than 30 sessions, specialty for whom the number of counseling sessions is 30 to 90, master for whom the number of counseling sessions is 90 or more, etc.), a “Please select the topic you would like to discuss together” item (e.g., including “I have concerns about myself” (e.g., including self-esteem, personality/disposition, value, interest, religion, background, marriage, etc.), “Social life is difficult,” “Interpersonal relationships are difficult,” “I need dream/career advice,” “I want to know about my emotions/psychology,” “I need expert advice,” etc.), a “Please select the situation in which you want to say something well” item (e.g., including a situation of expressing oneself, a work situation, a situation of presenting in front of others, a situation of dealing with a difficult subject, a situation that promotes friendship, a situation where there is conflict with others, a situation of having to express honest feelings, a discussion/persuasion situation, etc.), a “Please select the mentoring field you would like to receive mentoring in” item (e.g., including mentoring about oneself, mentoring for social life, mentoring for dream/careers, etc.) etc. Here, the “Please select the topic you would like to discuss together” item may include “me,” social life, interpersonal relationships, dreams/career, emotions/psychology, experts, etc., and the “Please select the situation in which you want to say something well” item may include “expressing myself,” work situation, presentation situation, difficult object, friendship, conflict situation, emotion expression, discussion/persuasion, etc.

For example, when a search menu (or expert search menu) 760 is selected from the “free expression” app execution result screen 700, the first terminal displays the expert search screen corresponding to the selected search menu 760 (S530).

Thereafter, the terminal 100 receives expert search information including a plurality of other input values corresponding to a plurality of other input items according to the user input of the terminal 100 from the expert search screen displayed on the terminal 100. Here, the expert search information includes an expert type, an expert gender, an expert level, “Please select a topic you would like to talk about (or topic you would like to receive psychological counseling on),” “Please select a situation in which you want to say something well (or speech lesson topic),” “Please select a mentoring field you would like to receive mentoring in (or mentoring topic),” desired counseling date and visual information, etc.

In addition, the terminal 100 transmits the received expert search information, the identification information of the terminal 100, etc., to the server 200.

As an example, the first terminal receives first expert search information including a first expert type (e.g., psychological counseling expert), a first expert gender (e.g., female), a first expert level (e.g., on a grade of 1 to 5, top 1), a first topic I want to talk about (for example, I want to restore my self-esteem), first desired counseling date and time (e.g., Dec. 5, 2021, 3 p.m.), etc., according to the first user input on the expert search screen displayed on the first terminal.

In addition, the first terminal transmits the received expert search information, the identification information of the first terminal, etc., to the server 200 (S540).

Thereafter, the server 200 receives the expert search information, the identification information of the terminal 100, etc., transmitted from the terminal 100.

In addition, the server 200 searches for (or confirms) an expert corresponding to the expert search information among one or more experts registered in the server 200.

As the search results (or the confirmation results), when the expert corresponding to the expert search information is found among one or more experts registered in the server 200, the server 200 transmits one or more pieces of expert information corresponding to the found expert search information to the terminal 100. Here, the expert information includes an expert name, a counseling field (or specialty field) (e.g., psychological counseling, speech lesson, mentoring, etc.), a brief introduction, a photo (or image), a fee per session, a rating (or star rating), review information, immediate counseling availability, popularity (or number of likes), etc.

In addition, the terminal 100 receives one or more pieces of expert information corresponding to the expert search information transmitted from the server 200 in response to the previously transmitted expert search information, and displays (or outputs) the received one or more pieces of expert information. Here, the expert information includes an expert name, a counseling field (or specialty field) (e.g., psychological counseling, speech lessons, mentorship, etc.), a brief introduction, a photo (or image), a fee per session, a rating (or star rating), review information, immediate counseling availability, popularity (or number of likes), etc.

As an example, the server 200 receives the first expert search information, the identification information of the first terminal, etc., transmitted from the first terminal.

In addition, the server 200 searches for (or confirms) an expert corresponding to the expert search information among one or more experts registered in the server 200.

In addition, the server 200 transmits the first to third expert information corresponding to the found corresponding expert search information to the first terminal.

In addition, the first terminal receives the first to third expert information transmitted from the server 200 in response to the previously transmitted first expert search information, etc.

In addition, the first terminal displays the received first to third expert information (S550).

Thereafter, when the specific expert information is selected from one or more pieces of expert information displayed on the terminal 100, the terminal 100 is linked with the server 200 to display the detailed expert information corresponding to the selected specific expert information. Here, the detailed expert information includes an expert name item, a counseling field (or specialty) item (e.g., including psychological counseling, speech lesson, mentoring, etc.), a rating (or star rating) item, a photo (or image) item, a brief introduction item, a usage fee per counseling session, a re-use desire rate of users who received counseling item, a total number of counseling sessions item, a counseling information item, a counselor information item, a review item, etc. In addition, the counseling information item includes a price option item (e.g., including a basic counseling item, a severe anxiety counseling item, etc.), a detailed description item, a location item, a popularity (or number of likes) item, a counseling application item, etc. In addition, the basic counseling item, the severe anxiety counseling item, etc., include the number of counseling sessions, additional composition (e.g., XR chat room training 40%, data analysis 60%, etc.), time (e.g., including 1 hour, etc.), price (e.g., 50,000 won/time), etc. In addition, the counselor information item includes a counselor introduction, a career introduction, a certification introduction, etc. In addition, the review item includes an overall star rating, ratings for each category (e.g., including use of XR, friendliness, analytical skills, empathy and comfort, appropriate advice, etc.), the total review, etc.

For example, when the first expert information is selected from the first to third expert information displayed on the first terminal, the first terminal is linked with the server 200 to display first expert detailed information related to the selected first expert information (S560).

Thereafter, when the preset counseling reservation menu displayed on one side of the expert detailed information of the terminal 100 is selected, the terminal 100 is linked with the server 200 and the payment server (not illustrated) to perform the payment function for the preset counseling cost related to the expert. In this case, the server 200 may perform card payment, an automatic transfer function through linkage with a bank payment account, payment using cash points or cash remaining in the account of the terminal 100 that is subscribed as a member of the server 200, and a payment function through simple payments including Kakao Pay, Naver Pay, etc. Here, before/after performing the payment function for the counseling fee, the terminal 100 may schedule the counseling with the corresponding expert, or may automatically reserve (or confirm) the counseling schedule with the expert according to the desired counseling date and time information included in the expert search information.

As an example, when the counseling reservation menu displayed on one side of the first detailed expert information of the first terminal is selected, the first terminal is linked with the server 200 and the payment server to perform the payment function for the first counseling fee (e.g., 50,000 won per hour) preset in relation to the first expert and confirm the counseling schedule with the first desired counseling date and time information (e.g., 3 p.m. on Dec. 5, 2021) (S570).

Thereafter, according to the corresponding reserved counseling schedule for the expert terminal 300, when the terminal 100 is invited to the metaverse chat room (or XR counseling room/XR chat room) created by the expert terminal 300, the terminal 100 enters (or participates in) the metaverse chat room (or XR counseling room/XR chat room) created by the corresponding expert terminal 300. In this case, the terminal 100 displays the metaverse waiting screen according to the entry into the metaverse chat room. Here, the metaverse waiting screen includes the type of the corresponding metaverse chat room, the topic, the chat room name, the keyword, the nickname (or chat name) of the user in the corresponding metaverse chat room, the chat temperature, and the number of chats (or the number of times the user has participated in the chat room), etc. In this case, the chat temperature may be calculated based on the number of chats, the received evaluation results, etc., and the concept of level or points may be applied instead of the chat temperature. That is, for the level or points, the level or the preset number of points may be calculated according to a plurality of preset grades based on the number of chats, the received evaluation results, etc.

As an example, when the first terminal is invited to the self-esteem recovery metaverse chat room created by the third expert terminal 300, as illustrated in FIG. 78, the first terminal participates in the self-esteem recovery metaverse chat room and displays the self-esteem recovery metaverse chat room screen (or self-esteem recovery metaverse chat room waiting screen) 7800 corresponding to the corresponding self-esteem recovery metaverse chat room (S580).

Thereafter, the terminal 100 is linked with the server 200 and the expert terminal 300 to receive the questionnaire transmitted (or provided) from the expert terminal 300 via the server 200. Here, the questionnaire includes one or more multiple-choice/subject questions related to a specific situation (or specific topic).

In addition, the terminal 100 displays the received questionnaire.

As an example, the first terminal receives the self-esteem self-diagnosis test questionnaire transmitted from the third expert, and displays the received self-esteem self-diagnosis test questionnaire 8000, as illustrated in FIGS. 79 and 80 (S590).

Thereafter, the terminal 100 receives questionnaire response information including a plurality of input values corresponding to the plurality of input items included in the questionnaire displayed on the terminal 100. Here, the questionnaire response information includes response information (e.g., not at all, no, average, yes, very much, etc.) for each question.

In addition, the terminal 100 transmits the received questionnaire response information, the identification information of the terminal 100, etc., to the expert terminal 300 via the server 200.

As an example, the first terminal receives the eleventh input value including the first to fifth response values which are response values corresponding to the first to fifth questions corresponding to the plurality of input items included in the self-esteem self-diagnosis test questionnaire 8000 displayed on the first terminal according to user input.

In addition, the first terminal transmits the eleventh input value related to the received self-esteem self-diagnosis test questionnaire, the identification information of the first terminal, etc., to the third expert terminal via the server 200.

Thereafter, the terminal 100 receives the response results to the questionnaire related to the corresponding questionnaire response information transmitted (or provided) from the expert terminal 300 in response to the questionnaire response information related to the previously transmitted questionnaire. Here, the response results include the user's response information for each question, correct answer information for each question, and the user's earned score compared to the total points, etc.

In addition, the terminal 100 displays (or outputs) the response results to the received questionnaire.

As an example, the first terminal receives the eleventh response result transmitted from the third expert terminal via the server 200 in response to the eleventh input value, etc., related to the previously transmitted self-esteem self-diagnosis test questionnaire, and display the received eleventh response result (S610).

Thereafter, the terminal 100 is linked with the server 200 and the expert terminal 300 to perform the psychological counseling function, the speech lesson, the mentor function, etc., with the expert through the metaverse chat room (or counseling room/XR chat room) generated from the corresponding expert terminal 300.

In addition, after the chat function ends, the terminal 200 receives the feedback information transmitted from the expert terminal 300, and displays (or outputs) the received feedback information. Here, the feedback information includes information such as an expert's view (or opinion) regarding the psychological counseling function, the speech lesson function, the mentor function, etc., performed in the metaverse chat room.

As an example, the first terminal is linked with the server 200 and the third expert terminal to perform the psychological counseling function in the self-esteem recovery metaverse chat room, receives the twelfth feedback information according to the performance of the psychological counseling function, and displays the received twelfth feedback information (S620).

According to the present invention, as described above, when the preset theme practice menu is selected from the app execution result screen, in situations where there is a spectator or in situations where there is no spectator focusing on situations in which the user of the terminal shows interest, by performing the group counseling or group training through the metaverse chat room with the free theme or discussion theme, it is possible to easily confirm the user's psychological state in relation to the specific situation in which the user is interested.

In addition, according to the present invention, as described above, when the preset “my report” menu is selected from the app execution result screen, by providing the analysis results according to the performance of functions such as topic talk, role-playing, and theme practice in relation to the user of the terminal, and the participation list related to the chat list in which the corresponding user participates, it is possible for the user to easily confirm the user's psychological state for each situation and the user's overall psychological state in relation to chat.

In addition, according to the present invention, as described above, when the preset topic talk menu is selected from the app execution result screen, by performing the chat function with other users of terminals focusing on the specific topic in which the user of the terminal is interested and the keywords related to the specific topic, and providing the chat results based on the self-evaluation and counterpart evaluation, it is possible to easily confirm the user's psychological state in relation to the specific topics in which the user is interested and the keywords.

In addition, according to the present invention, as described above, when the preset role-playing menu is selected from the app execution result screen, by performing the chat function according to role-playing with other users of terminals focusing on the specific topic in which the user of the terminal is interested and the keywords related to the specific topic, and providing the chat results based on the self-evaluation and counterpart evaluation, it is possible to easily confirm the user's psychological state in relation to the specific topics in which the user is interested and the keywords.

In addition, according to the present invention, as described above, when the preset theme practice menu is selected from the app execution result screen, by providing the feedback related to the situations in which the user of the terminal is interested either alone or in collaboration with other users of terminals and providing the chat results based on the self-evaluation and counterpart evaluation according to the feedback, it is possible to easily confirm the user's psychological state in relation to the specific situation in which the user is interested.

In addition, according to the present invention, as described above, when the preset people diary menu is selected from the app execution result screen, by recording the emotions felt by the user of the terminal in the specific situation during the day in a diary format, it is possible to conveniently organize the change in the user's emotional state by date.

The above-described content may be variously modified and altered by those skilled in the art to which the present invention pertains without departing from the essential features of the present invention. Accordingly, exemplary embodiments disclosed in the present invention are not to limit the spirit of the present invention, but are to describe the spirit of the present invention. The scope of the present invention is not limited to these exemplary embodiments. The scope of the present invention should be interpreted by the following claims and it should be interpreted that all spirits equivalent to the following claims fall within the scope of the present invention.

### Mode for Invention

Modes for carrying out the invention have been described together in the best mode for carrying out the present invention described above.

### Industrial Applicability

The present invention has industrial applicability in that, when a preset theme practice menu is selected from an app execution result screen, in situations where there is a spectator or in situations where there is no spectator focusing on situations in which a user of a terminal shows interest, by performing group counseling or group training through a metaverse chat room with a free theme or discussion theme as a topic, it is possible to easily confirm a user's psychological state in relation to a specific situation in which the user is interested.

1. A system for providing psychological counseling, comprising:
a terminal that displays a room creation screen corresponding to a selected room creation menu when a preset room creation menu is selected from an app execution result screen displayed on the terminal, displays a spectator type setting screen corresponding to a selected theme practice item when a preset theme practice item is selected from the room creation screen displayed on the terminal, displays a theme setting screen corresponding to a selected no spectator item when a preset no spectator item is selected from the spectator type setting screen displayed on the terminal, displays a questionnaire setting screen for receiving self-introduction or question content when a preset question-and-answer item is selected from the displayed theme setting screen, displays a detailed theme setting screen to which received questionnaire setting information is applied when a registration item displayed on one side of the questionnaire setting screen is selected after receiving the questionnaire setting information including a plurality of input values corresponding to a plurality of input items included in the questionnaire setting screen displayed on the terminal, and is linked with a server to create a new question-and-answer theme practice chat room corresponding to received detailed theme setting information when a room creation item displayed on one side of the detailed theme setting screen is selected after receiving the detailed theme setting information including a plurality of other input values corresponding to a plurality of other input items included in the detailed theme setting screen displayed on the terminal; and
the server that manages a chat function in the question-and-answer chat room related to the theme practice.

2. The system of claim 1, wherein a waiting screen includes at least one of a type of the chat room, a topic, a chat room name, a keyword, a nickname of a user in the chat room, a level, and the number of chats, and
the level is calculated based on the number of chats and the received evaluation results.

3. The system of claim 1, wherein the terminal enters the created question-and-answer theme practice chat room to display a waiting screen, and when a room leader who opens the corresponding chat room selects a preset start menu while the number of people set in the created question-and-answer theme practice chat room is met, is linked with one or more other terminals owned by each of one or more other users in the question-and-answer chat room related to the theme practice and the server to perform a chat function in relation to a specific theme of the question-and-answer chat room.

4. A method of providing psychological counseling, comprising:
when a preset room creation menu is selected from an app execution result screen displayed on a terminal, displaying, by the terminal linked with a server, a room creation screen corresponding to the selected room creation menu;
when a preset theme practice item is selected from the room creation screen displayed on the terminal, displaying, by the terminal linked with the server, a spectator type setting screen corresponding to the selected theme practice item;
when a preset no spectator item is selected from the spectator type setting screen displayed on the terminal, displaying, by the terminal linked with the server, a theme setting screen corresponding to the selected no spectator item;
when a preset question-and-answer item is selected from the displayed theme setting screen, displaying, by the terminal, a questionnaire setting screen for receiving self-introduction or question content;
after receiving questionnaire setting information including a plurality of input values corresponding to a plurality of input items included in the questionnaire setting screen displayed on the terminal, when a registration item displayed on one side of the questionnaire settings screen is selected, displaying, by the terminal, a detailed theme setting screen to which the received questionnaire setting information is applied;
after receiving detailed theme setting information including a plurality of other input values corresponding to a plurality of other input items included in the detailed theme setting screen displayed on the terminal, when the room creation item displayed on one side of the detailed theme setting screen is selected, creating, by the terminal linked with the server, a new question-and-answer theme practice chat room corresponding to the received theme detailed setting information and entering the created question-and-answer theme practice chat room to display a waiting screen; and,
when a room leader who opens the corresponding chat room selects a preset start menu while the number of people set in the generated question-and-answer theme practice chat room is met, performing, by the terminal linked with one or more other terminals owned by each of one or more other users in the question-and-answer chat room related to the theme practice and the server, a chat function in relation to a specific theme of the question-and-answer chat room.

5. A method of providing psychological counseling, comprising:
when a preset room creation menu is selected from an app execution result screen displayed on a terminal, displaying, by the terminal linked with a server, a room creation screen corresponding to the selected room creation menu;
when a preset theme practice item is selected from the room creation screen displayed on the terminal, displaying, by the terminal linked with the server, a spectator type setting screen corresponding to the selected theme practice item;
when a preset spectator item is selected from the spectator type setting screen displayed on the terminal, displaying, by the terminal linked with the server, other theme setting screens corresponding to the selected spectator item;
when any one preset theme item is selected from the displayed other theme setting screens, displaying, by the terminal, other detailed theme setting screens for receiving a room title, the number of participants in training, or the maximum number of spectators;
after receiving other detailed theme setting information including a plurality of other input values corresponding to a plurality of other input items included in the other detailed theme setting screens displayed on the terminal, when the room creation item displayed on one side of the other detailed theme setting screens is selected, creating, by the terminal linked with the server, a new free theme chat room or a discussion theme chat room corresponding to the received other theme detailed setting information and entering the created free theme chat room or discussion theme chat room to display a waiting screen; and,
when a room leader who opens the free theme chat room or the discussion theme chat room selects a preset start menu while the number of people set in the created free theme chat room or discussion theme chat room is met, performing, by the terminal linked with one or more other terminals owned by each of one or more other users in the free theme chat room or the discussion theme chat room related to the theme practice and the server, a chat function in relation to a specific theme of the free theme chat room or the discussion theme chat room.

6. The method of claim 4, further comprising:
when the theme practice related to the question-and-answer chat room ends, performing, by the terminal linked with the server, a self-evaluation function for the user of the terminal itself;
generating, by the server, a chat result for the user of the terminal according to the self-evaluation function performed on the terminal, and transmitting the generated chat result to the terminal; and
displaying, by the terminal, the chat result transmitted from the server.

7. The method of claim 6, further comprising:
updating, by the server, a “free expression” index, an anxiety index, and a level related to the user based on the chat result generated in relation to the user of the terminal

8. The method of claim 7, wherein, in relation to one or more chat rooms in which the user of the terminal participates for a preset period or all chat rooms in which the user of the terminal participates, the “free expression” index is calculated by adding up an average score self-evaluated by the user after performing the chat function in the chat room in which the user participates and results of a counterpart's evaluation of the user in the chat room in which the user participates, and
the anxiety index represents an average value of anxiety self-evaluated by the user after performing the chat function in the chat room in which the user participates.

9. The method of claim 4, further comprising:
when the theme practice related to the free theme chat room or the discussion theme chat room ends, performing, by the terminal linked with the server, a self-evaluation function on the user of the terminal itself and a counterpart evaluation function on one or more other users participating in the free theme chat room or the discussion theme chat room;
generating, by the server, chat results for each user participating in the chat room and transmitting each of the generated chat results for each user to the terminal depending on the performance of the self-evaluation function and the counterpart evaluation function performed by each of the plurality of terminals participating in the free theme chat room or the discussion theme chat room; and
displaying, by the terminal, the chat result transmitted from the server.

10. A method of providing psychological counseling, comprising:
when a terminal is invited to a metaverse chat room created in an expert terminal, entering, by the terminal, the metaverse chat room to display a waiting screen;
displaying, by the terminal, a questionnaire transmitted from the expert terminal via a server;
receiving, by the terminal, questionnaire response information including a plurality of input values corresponding to a plurality of input items included in the questionnaire displayed on the terminal, and transmitting the received questionnaire response information to the expert terminal;
displaying, by the terminal, a response result to a questionnaire related to the questionnaire response information transmitted from the expert terminal in response to questionnaire response information related to the previously transmitted questionnaire;
performing, by the terminal linked with the server and the expert terminal, one of a psychological counseling function, a speech lesson function, and a mentor function with an expert through a metaverse chat room created by the expert terminal; and
displaying, by the terminal, feedback information transmitted from the expert terminal after a chat function ends.
